SEQUENCE LISTING (13 sequences)

SEQ ID NO: 1 - DNA, 3749 bp, Brassica rapa
  CDS (1)..(3675)
atgaaatttcaatcgtttttgaaagaaattaagaaacagagagggaagaggaaaagagatgaactttcgaatccagcaagaaagaagatccgagttcagaacctgagccaaatccaagtaccatcatcacccccaccttctccttcatcacttccatcctctttgtctctttcatcagctccatcttcttcgtctcataactggacacacgatgtctttccaagcttccgcggggaagatgtccgcatagggtttctaagccacattcaaaaggagtttaaaagaaaaggaatcacaccattcatcgacaatgagatcaggagaggagaatccatcggtccagaactcatacgggccattagaggatctaaaatcgccatcgtcttgctctcgaggaactatgcttcttcaaagtggtgtcttgatgagttggtggaggttatgaagtgcaaagaagagttaggccaaaccgtgatccccgttttctataaagtagatccttctcatgtaaagaagctgagaggatattttgggaaagttttcgaaaaaacttgcgagggtaaaagtaaggaggatactgagaaatggagacatgctttggagaaggtggccacaattgctggttacgattcaagcacctgggataatgaagcggccatgattgagcaaatagccacagatgtttcaaacaagctgattagttctgttccatcaagtgatttcaacagcttagttgggatgagagctcacatgaaaagtatggaactgctcttacgcttggactccgatgaagtgaggatgatagggatttggggtccgtctggaattggtaagagcaccatcgccagatctctctttagccaacactctcctgactttcaacttagcgtcttcatggagaatatcaaaagagagtatccgagaccttgtttcgatagatacagcgcacaagtgcaattacaaaataagttcttgtctctaatactcaatcagaatgatgtcgctatccatcatttaggagttgcacaagaccggctaaaaaataagaaagtgttagttgttctcgatgacgtggatcactcagcgcaactagatgccttggcgaaagaaacttgctggtttggcagtggaagtcggattatcgtcacgacgcaagataagaaaattttgaatgcacatcggatcaatcatatttacgaggttggttttccacatgatgatgaagctcttgaaatcttctgcataaatgcttttggtcaaaaatccccatatgatggttttggggaccttgctcgggaagttacaaggcttgtgggtaacctccctttgggactaagtgttatgggatcttatttcaaaggcttgtccaaggaggtgtgggaacgtgagttaccaaggttaagaactagacttgacggtgaaacagaaagtattttaaagttcagctatgatgccttatgcgatgaagatcaagctttgtttcttcacatagcctgctttttcaacggtgaacggactgacaaagtagaagagtttcttgcagagaaatttgttgctgtggaaggtcgtcttcgtgttttagctgagaaatctctcatatccgtcggctcagaaggatatataaggatgcatgatttgctagcacgtttgggtagggaaattgttcgtaaacaatcccctaacgaacctgggcagcgtcagtttttggttgatgatggagatatacgccaagtactacgtgatgatacacttggtagtcgaagtgttataggaataaattttttgttgaagaagaaattgaagataagtgaccaagcctttgaaagaatgtccaatctccaattcttaagacttgatagtcagtattttgcccagattctattcgaaggaaaaagtagtcaatacatattagaaagtgtgaactgtctacctcgagaagttagattactagattggagaacattcccgatgacatgtctgccttctgattttaatccagagctcctaatggaaataaaaatgatttgtagcaaccttgagaaattgtgggaaggaaataaaacgattagaaatctcaagtggatggatctgtctcattccaaaaatctaaaggagcttcctaatctttcaactgccacaaatctccgagaactgaatctctttggatgttcaagtcttatggagcttccttcttctattgggaatttgactaatctcaagaaattgaatctcaagctgtgctcaagccttatggaactcccctcttctattggtaacatgactaatcttgagaatttgaatctttctggatgctctagccttgtggagctcccctcttctattagtaatatgactaatcttgagaattttaatctctcccaatgctcaagcgttgtacggctctctttttctattggaaatatgaccaatctcaaggaattggagctcaatgaatgctcaagccttgtggagctcacctttgggaatatgactaatctcaagaatttggatcccaatagatgctcaagccttgtggagatttcctcttctattgggaatatgactaatctcgtgaggttggatctcaccggatgctcaagccttgtggagctcccctattctattgggaatatgactaatcttgaaactttggaactctctggatgttcaagccttgtggagctcccctcttctattgggaatcttcataatttgaagcggttgaatctcagaaattgctcaacgctaatggcccttccagtgaacatcaacatgaaatctcttgattttcttgatctcagttactgctcggtgttgaaaagctttcctgagatttccacaaacattatctttctaggaatcaaaggaactgccattgaagaaattccaacatcaatcaggtcatggtctcgtcttgatacgttagatatgtcatacagtgaaaacctaaggaaatcgcatcatgcttttgacctcatcacgaatcttcacttgagcgacacaggaattcaagaaatttctccatgggtaaaagaaatgtctcgtctacgggaacttgtaatcaatggatgcacaaagctggtttcgctcccacagcttccagattcattagaattcatgcatgtagaaaactgcgagtccctcgagagactagatagtctagattgctctttttacaggacaaagttgactgatcttcgctttgttaactgcctcaaactgaatcgagaagcagtagaccttattctcaagacatcgacaaaaatatgggcgatctttcccggagaatcggtgcctgcatatttcagttacagagccacggggagttcagtgtcaatgaaactgaatagattcgatacacgttttcctacatccttgagatttaaagcttgcatcttgctggttactaaccctgacgacgttgagcctgctgcttggtacaggtcggatatgtcttattgcatcaatggcaaactgagggatgccggtgttttcctagcatatactcatatatgggacccactccgtccacgttctgagcatctggtcgtaatcgaatttgaagaaactgtgacttcccccgaattagtctttgagttcaggttcgaaaaagaaaactgggagattaaagaatgcggactacgtcctctagaaagcttagctctctcatgttaatggaagctgaagaaacatttcgcatgtgcataaaatatagtaatgcattccgttgtttatcttaaatatcaagg

SEQ ID NO: 2 - protein, 1224 aa, Brassica rapa
MKFQSFLKEIKKQRGKRKRDELSNPARKKIRVQNLSQIQVPSSPPPSPSSLPSSLSLSSAPSSSSHNWTHDVFPSFRGEDVRIGFLSHIQKEFKRKGITPFIDNEIRRGESIGPELIRAIRGSKIAIVLLSRNYASSKWCLDELVEVMKCKEELGQTVIPVFYKVDPSHVKKLRGYFGKVFEKTCEGKSKEDTEKWRHALEKVATIAGYDSSTWDNEAAMIEQIATDVSNKLISSVPSSDFNSLVGMRAHMKSMELLLRLDSDEVRMIGIWGPSGIGKSTIARSLFSQHSPDFQLSVFMENIKREYPRPCFDRYSAQVQLQNKFLSLILNQNDVAIHHLGVAQDRLKNKKVLVVLDDVDHSAQLDALAKETCWFGSGSRIIVTTQDKKILNAHRINHIYEVGFPHDDEALEIFCINAFGQKSPYDGFGDLAREVTRLVGNLPLGLSVMGSYFKGLSKEVWERELPRLRTRLDGETESILKFSYDALCDEDQALFLHIACFFNGERTDKVEEFLAEKFVAVEGRLRVLAEKSLISVGSEGYIRMHDLLARLGREIVRKQSPNEPGQRQFLVDDGDIRQVLRDDTLGSRSVIGINFLLKKKLKISDQAFERMSNLQFLRLDSQYFAQILFEGKSSQYILESVNCLPREVRLLDWRTFPMTCLPSDFNPELLMEIKMICSNLEKLWEGNKTIRNLKWMDLSHSKNLKELPNLSTATNLRELNLFGCSSLMELPSSIGNLTNLKKLNLKLCSSLMELPSSIGNMTNLENLNLSGCSSLVELPSSISNMTNLENFNLSQCSSVVRLSFSIGNMTNLKELELNECSSLVELTFGNMTNLKNLDPNRCSSLVEISSSIGNMTNLVRLDLTGCSSLVELPYSIGNMTNLETLELSGCSSLVELPSSIGNLHNLKRLNLRNCSTLMALPVNINMKSLDFLDLSYCSVLKSFPEISTNIIFLGIKGTAIEEIPTSIRSWSRLDTLDMSYSENLRKSHHAFDLITNLHLSDTGIQEISPWVKEMSRLRELVINGCTKLVSLPQLPDSLEFMHVENCESLERLDSLDCSFYRTKLTDLRFVNCLKLNREAVDLILKTSTKIWAIFPGESVPAYFSYRATGSSVSMKLNRFDTRFPTSLRFKACILLVTNPDDVEPAAWYRSDMSYCINGKLRDAGVFLAYTHIWDPLRPRSEHLVVIEFEETVTSPELVFEFRFEKENWEIKECGLRPLESLALSC

SEQ ID NO: 3 - DNA, 7995 bp, Brassica rapa
  exon (2525)..(3165)
  Intron (3166)..(4652)
  exon (4653)..(5763)
  Intron (5764)..(5856)
  exon (5857)..(6165)
  Intron (6166)..(6264)
  exon (6265)..(7875)
accaaagagattgtgctgcagagattcttttcttgggatccttactgagcgtttttcataacaaggtcatggttggaaggtccagcgatatccctgtaagcttcatatcaagcatatgaacacccgagattattatcaactaagactaccataattgagaatctgttttctttttttcagtggagcaggatatgtttctcttattgagtatgctcatttaactggaagacctttctgacttattgagtacatgtttcctgatgctgttgagatccaagctttggttaggaacaaggtgtttgtgtctctttctctcaagatggtcaccaagagtgttgcctatccgaaaagtgagtccgctgaggcgtgccttaaagggtttatttaaagttgattttagcatcactttatcatcgatagtttcttgcggcaccatctcattctaatggaagctgaaaaaaacaattcgcagtttgctgtatctgtgtaaactactttctgtaaaacatttcccccggtttaggacagcaatacttctacttccactataacttctagaataatgtttttggatcaagtttaattttatgttcctccttttcgatttatcaatgaatggaatggaacaagttttcccttttgtggctgcgtggtaccagtatcacttcatcatggcacagaattcttcattgttaatctcaacgttggctcataggtttttttcatgctatgtaatgtagtgggaaacataacttgtaattatctgtatctacttcctctatagcttctttgatgatagcttcatctcccataccatactatttcatggctaactccaactcatccattgttgataaacctgtgtaagccaattccatctcagtgtctcggaatattgttttaacttctcattctatagttttctctgttccaccaaagagaacttaccagctcttgtctttgtcaaagtattggaagtctttgaacaagtgctcctctcaatccagtctgtatctctagtaataacttccatgagttggttaacttcatctacagagagtctagaaccgtgtttagccaccctggttttcagttctttgtaattaagggaggtacaccacaaagcaagatgtatagaataatctctgcactccaaatatctctgtaaacttattgaagcccataaaaaagacccattgaatgatccattaagaagcttttgtaaccatgtaactagggtttatagaccattataaatatgtccttagatgttctttggttgtaactttggctgctataatagcagcctctccctttccattatatgaaatcaaatccaacgtttatctcctggtgacaacttaatctcttcctctctttagtttctagcatttcaataaagctttcttaagctatctttcatgtgtcaataaagcttagcaaagctataatcttttatgtttctcaaacgttatcaaaactttccctgcaattttgttttcagtttataaacaacacataaagcttatgaatacacagttatcaactaacaaaaaagaaaacacaaggtctacacaagtaattactcacattgccaacttgcaaagcctctttttgaagattaccactatgtactgaactaataagactctcaagttatatatggatcaaacctgaaaataaacattggttataatcttaagaaatctaaaacatagcatttacatatatcttataaccagaagagttgtatgtatagcgttctcaacttctccaatgaaaacagacaatccgaagtcagtagctttgagcatagcgttctcatctttactaaagagcaagaagttctcaggcttgatctacaatcctatcggacaattcacttccaccaccacacatttccattacaagacgaatggactgtctagatgatgatgaccagatgttcatgaacattattttttatgaagaatgtactttttgaatgaatatgaaaaccaaacgaagcccaaacgttttaaaataaaagattggtatgagtaggaggagaaatatataaaaaagcttctagagaagattgaatgatctctttgctgtgaaattttcaagtgcgaactcatcagttctgatcactgcttctttgaaattgcttgtgctcttccttgcattttccttcttagaatagtataagccaaaagggaatggtgggactttatgcaccttatttttcgttggtgacatatttttccacaaaagtctttgtaagtcggccgagtcaacgttttatgacatatacgcaattacgcatacaaaattgtcacaagtctctttggttcattattgcgttacatatcttctttagaaatattcttggtccctctctctctaccacacagattgattatcccttctaatcatcccctctacaattcttgctgtatgcttcagattccatcaagaaaaccaagaaactgattttctttttttttcaacttcggtaaaaaaaaaaggcagctttttgaaaatgaaatttcaatcgtttttgaaagaaattaagaaacagagagggaagaggaaaagagatgaactttcgaatccagcaagaaagaagatccgagttcagaacctgagccaaatccaagtaccatcatcacccccaccttctccttcatcacttccatcctctttgtctctttcatcagctccatcttcttcgtctcataactggacacacgatgtctttccaagcttccgcggggaagatgtccgcatagggtttctaagccacattcaaaaggagtttaaaagaaaaggaatcacaccattcatcgacaatgagatcaggagaggagaatccatcggtccagaactcatacgggccattagaggatctaaaatcgccatcgtcttgctctcgaggaactatgcttcttcaaagtggtgtcttgatgagttggtggaggttatgaagtgcaaagaagagttaggccaaaccgtgatccccgttttctataaagtagatccttctcatgtaaagaagctgagaggatattttgggaaagttttcgaaaaaacttgcgagggtaaaagtaaggaggatactgagaaatggagacatgctttggagaaggtggccacaattgctggttacgattcaagcacctggtttgtccttttctatttttctttttatttctaatctttcaaactcatacaaatctaactcaaaaataccaaatgatcacagttttttcctcttaatttttacttgctctttatatgcatcaatatataatatcaatgttaaatctcaaaatataacaaaaaggtgttagttccaaaaattcaaaaacttgtgaaaacttatacggaaaaaagtttcctagctacacgaagtttacactaatatggtggaaaccatccagtgtatgagaatgtgtcaacaactacacgaagtttatgttaatacatgtcacatgtacgaactaaataataatatggtggcaacatcacaatcggttttaattgattttaaactaagttaatggggtcaatccgtaatttactaaacttaataaatttaatatttttttttaattttaataaacttaattgattttgttaataaacttaataattaattaaacttacttaataaacttaataaatttactaaacttataaaaattaataaatttattaaacttataaaaattaataaatttattaaacttaataaaattaatcatttattaataaacttaatcatttattaataaatttaataaaattaatcatttattaataaacttaatcatttaataaacttaataaacctaataaatttgttaaatttatcaagtttaataagtttattaagtttaataaatttatttttattaagtttaagtttattaagtttattaaattattaagtttattaagtttaatgaatttattaagtataataaattattaagtttattaagttgaattttattaaattaaaaagtttattaataaaatcaattaagtttattaaacttaataaaccattaagtttattaagtttattaaattataaagtttattaagtttaataaatttattaagtataataaattattaagttttttaagttaaagtttattaaattactaagtttattaataaaatcaatttaagtttattaaacttaataaatcattaagtttattaagtttagtaaattacagattgaccccattaacttagcttataatcaattaaacccgattgtgatgttgccaccatattattatttacttcgtacatgtaacatgtattaacataaacttcgtgtagttgttgacacattttcatatattggatagtttccaccatattagtgtaaacttcgtgtagctatgaaacttttttccaacttataccattctctaaattttgaacatgtttggttatttaatgaagagatattgggagcgtgaattcaaatagagttgcttaatttttaatttaagagttttgtgtatttaacatcatccaaaaacatgcaatgtgacattcttgagaattgcacacagcatatttccagccaaacaattcttatttggaaaccgtttgagccgatgtatgttctttcctctgtttagggataatgaagcggccatgattgagcaaatagccacagatgtttcaaacaagctgattagttctgttccatcaagtgatttcaacagcttagttgggatgagagctcacatgaaaagtatggaactgctcttacgcttggactccgatgaagtgaggatgatagggatttggggtccgtctggaattggtaagagcaccatcgccagatctctctttagccaacactctcctgactttcaacttagcgtcttcatggagaatatcaaaagagagtatccgagaccttgtttcgatagatacagcgcacaagtgcaattacaaaataagttcttgtctctaatactcaatcagaatgatgtcgctatccatcatttaggagttgcacaagaccggctaaaaaataagaaagtgttagttgttctcgatgacgtggatcactcagcgcaactagatgccttggcgaaagaaacttgctggtttggcagtggaagtcggattatcgtcacgacgcaagataagaaaattttgaatgcacatcggatcaatcatatttacgaggttggttttccacatgatgatgaagctcttgaaatcttctgcataaatgcttttggtcaaaaatccccatatgatggttttggggaccttgctcgggaagttacaaggcttgtgggtaacctccctttgggactaagtgttatgggatcttatttcaaaggcttgtccaaggaggtgtgggaacgtgagttaccaaggttaagaactagacttgacggtgaaacagaaagtattttaaagttcagctatgatgccttatgcgatgaagatcaagctttgtttcttcacatagcctgctttttcaacggtgaacggactgacaaagtagaagagtttcttgcagagaaatttgttgctgtggaaggtcgtcttcgtgttttagctgagaaatctctcatatccgtcggctcagaaggatatataaggatgcatgatttgctagcacgtttgggtagggaaattgttcgtaaacaatcccctaacgaacctgggcagcgtcagtttttggttgatgatggagatatacgccaagtactacgtgatgatacacttgtaagttttcacgttggttgttctcagcatttctcctagaaaatgttataacaaatcatgtttgcttatgtatctttgggttttttttttcagggtagtcgaagtgttataggaataaattttttgttgaagaagaaattgaagataagtgaccaagcctttgaaagaatgtccaatctccaattcttaagacttgatagtcagtattttgcccagattctattcgaaggaaaaagtagtcaatacatattagaaagtgtgaactgtctacctcgagaagttagattactagattggagaacattcccgatgacatgtctgccttctgattttaatccagagctcctaatggaaataaaaatgatttgtagcaaccttgagaaattgtgggaaggaaataaagtaagtacagatatatatatatatatatatatttcaaaataatatgctttagctgaggatcgtctttttcttaatgttgcattttgtttatctgtacagacgattagaaatctcaagtggatggatctgtctcattccaaaaatctaaaggagcttcctaatctttcaactgccacaaatctccgagaactgaatctctttggatgttcaagtcttatggagcttccttcttctattgggaatttgactaatctcaagaaattgaatctcaagctgtgctcaagccttatggaactcccctcttctattggtaacatgactaatcttgagaatttgaatctttctggatgctctagccttgtggagctcccctcttctattagtaatatgactaatcttgagaattttaatctctcccaatgctcaagcgttgtacggctctctttttctattggaaatatgaccaatctcaaggaattggagctcaatgaatgctcaagccttgtggagctcacctttgggaatatgactaatctcaagaatttggatcccaatagatgctcaagccttgtggagatttcctcttctattgggaatatgactaatctcgtgaggttggatctcaccggatgctcaagccttgtggagctcccctattctattgggaatatgactaatcttgaaactttggaactctctggatgttcaagccttgtggagctcccctcttctattgggaatcttcataatttgaagcggttgaatctcagaaattgctcaacgctaatggcccttccagtgaacatcaacatgaaatctcttgattttcttgatctcagttactgctcggtgttgaaaagctttcctgagatttccacaaacattatctttctaggaatcaaaggaactgccattgaagaaattccaacatcaatcaggtcatggtctcgtcttgatacgttagatatgtcatacagtgaaaacctaaggaaatcgcatcatgcttttgacctcatcacgaatcttcacttgagcgacacaggaattcaagaaatttctccatgggtaaaagaaatgtctcgtctacgggaacttgtaatcaatggatgcacaaagctggtttcgctcccacagcttccagattcattagaattcatgcatgtagaaaactgcgagtccctcgagagactagatagtctagattgctctttttacaggacaaagttgactgatcttcgctttgttaactgcctcaaactgaatcgagaagcagtagaccttattctcaagacatcgacaaaaatatgggcgatctttcccggagaatcggtgcctgcatatttcagttacagagccacggggagttcagtgtcaatgaaactgaatagattcgatacacgttttcctacatccttgagatttaaagcttgcatcttgctggttactaaccctgacgacgttgagcctgctgcttggtacaggtcggatatgtcttattgcatcaatggcaaactgagggatgccggtgttttcctagcatatactcatatatgggacccactccgtccacgttctgagcatctggtcgtaatcgaatttgaagaaactgtgacttcccccgaattagtctttgagttcaggttcgaaaaagaaaactgggagattaaagaatgcggactacgtcctctagaaagcttagctctctcatgttaatggaagctgaagaaacatttcgcatgtgcataaaatatagtaatgcattccgttgtttatcttaaatatcaaggaaacaatttttattagtaatctttgtttcctactcgttgagtg

SEQ ID NO: 4 - protein, 214 aa, Brassica rapa
MKFQSFLKEIKKQRGKRKRDELSNPARKKIRVQNLSQIQVPSSPPPSPSSLPSSLSLSSAPSSSSHNWTHDVFPSFRGEDVRIGFLSHIQKEFKRKGITPFIDNEIRRGESIGPELIRAIRGSKIAIVLLSRNYASSKWCLDELVEVMKCKEELGQTVIPVFYKVDPSHVKKLRGYFGKVFEKTCEGKSKEDTEKWRHALEKVATIAGYDSSTW

SEQ ID NO: 5 - protein, 341 aa, Brassica rapa
DNEAAMIEQIATDVSNKLISSVPSSDFNSLVGMRAHMKSMELLLRLDSDEVRMIGIWGPSGIGKSTIARSLFSQHSPDFQLSVFMENIKREYPRPCFDRYSAQVQLQNKFLSLILNQNDVAIHHLGVAQDRLKNKKVLVVLDDVDHSAQLDALAKETCWFGSGSRIIVTTQDKKILNAHRINHIYEVGFPHDDEALEIFCINAFGQKSPYDGFGDLAREVTRLVGNLPLGLSVMGSYFKGLSKEVWERELPRLRTRLDGETESILKFSYDALCDEDQALFLHIACFFNGERTDKVEEFLAEKFVAVEGRLRVLAEKSLISVGSEGYIRMHDLLARLGREIV

SEQ ID NO: 6 - protein, 106 aa, Brassica rapa
RKQSPNEPGQRQFLVDDGDIRQVLRDDTLGSRSVIGINFLLKKKLKISDQAFERMSNLQFLRLDSQYFAQILFEGKSSQYILESVNCLPREVRLLDWRTFPMTCLP

SEQ ID NO: 7 - protein, 284 aa, Brassica rapa
SDFNPELLMEIKMICSNLEKLWEGNKTIRNLKWMDLSHSKNLKELPNLSTATNLRELNLFGCSSLMELPSSIGNLTNLKKLNLKLCSSLMELPSSIGNMTNLENLNLSGCSSLVELPSSISNMTNLENFNLSQCSSVVRLSFSIGNMTNLKELELNECSSLVELTFGNMTNLKNLDPNRCSSLVEISSSIGNMTNLVRLDLTGCSSLVELPYSIGNMTNLETLELSGCSSLVELPSSIGNLHNLKRLNLRNCSTLMALPVNINMKSLDFLDLSYCSVLKSFPEI

SEQ ID NO: 8 - protein, 279 aa, Brassica rapa
STNIIFLGIKGTAIEEIPTSIRSWSRLDTLDMSYSENLRKSHHAFDLITNLHLSDTGIQEISPWVKEMSRLRELVINGCTKLVSLPQLPDSLEFMHVENCESLERLDSLDCSFYRTKLTDLRFVNCLKLNREAVDLILKTSTKIWAIFPGESVPAYFSYRATGSSVSMKLNRFDTRFPTSLRFKACILLVTNPDDVEPAAWYRSDMSYCINGKLRDAGVFLAYTHIWDPLRPRSEHLVVIEFEETVTSPELVFEFRFEKENWEIKECGLRPLESLALSC

SEQ ID NO: 9 - DNA, 33 bp, Artificial
  artificially synthesized primer sequence
tcccccgggaaaatgaaatttcaatcgtttttg

SEQ ID NO: 10 - DNA, 29 bp, Artificial
  artificially synthesized primer sequence
ccttgatatttaagataaacaacggaatg

SEQ ID NO: 11 - DNA, 24 bp, Artificial
  artificially synthesized primer sequence
ctctctcatgttaatggaagctga

SEQ ID NO: 12 - DNA, 24 bp, Artificial
  artificially synthesized primer sequence
cactcaacgagtaggaaacaaaga

SEQ ID NO: 13 - DNA, 12737 bp, Brassica rapa
accaaagagattgtgctgcagagattcttttcttgggatccttactgagcgtttttcataacaaggtcatggttggaaggtccagcgatatccctgtaagcttcatatcaagcatatgaacacccgagattattatcaactaagactaccataattgagaatctgttttctttttttcagtggagcaggatatgtttctcttattgagtatgctcatttaactggaagacctttctgacttattgagtacatgtttcctgatgctgtgctttggttaggaacaaggtgtttgttctctttctctcaagatggtcaccaagagtgttgcctatccgaaaagtgagtcctctgaggcgtgccttaaaggatttatttaaagttgaaccttaacaaatgttcaagccttgtggagcttccctcttctattgagaatatgactaatctccaaaatttatatctcaacggatgctcaggtctagagaagctcccctcttctattgggaatctttattgtttgcaaagattgcaaatgaaaggttgctcaaagcctaaattcccttccgattaccatcaatttgaaatctctttgggagctagatctcagatagtgctcgtcgttgaaaaggtttcctgagatttccataaatatttatagatctaaagcttaaaggagctgcactagaagaatttccatgattttagcatcactttatcatcgatagtttcttgcggcaccatctcattctatgggagctgaaaaaaacaattcgcagtttgctgtatctgtgtacactactttctgtaaaacatttcccccggtttaggacagcaatacttctacttccactataacttctagaataatgtttttggatcaagtttaattttatgttcctccttttcgatttatcaatgaatggaatggaacaagttttcccttttgtggctgcgtggtaccagtatcacttcatcatggcacagaattcttcattgttaatctcaacgttggctcataggtttttttcatgctatgtagtgggaaacataacttgtaattatctgtatctacttcctctatagcttctttgatgatagcttcatctcccataccatactatttcatggctaactccaactcatccattgttgataaacctgtgtaagccaattccatctcagtgtcttggaatattgttttaacttctcattctatagttttctctgttccaccaaagagaacttaccagctcttgtctttgtcaaagtattggaagtctttgaacaagtgctcctctcaatccagtctgtatctatagtaataacttccatgagttggttaacttcatctacagagagtctagaaccgtgtttagccaccctggttttcagttctttgtaattaagggaggtacaccacaaagcaagatgtatagaataatctctgcactccaaatatctctgtaaacttattgaagcccataagaaagacccattgaatgatccattaagaatcttttgtaaccatgtaactagggtttatagaccattataaatatgtcgttagatgttctttggttgtaagtctttggctggtataatagcagcctctccctttccaatatatgaaatcaaatccaacgtttatctcttggtgacaacttaatctcttcctctctttagtttctagcatttcaataaagctttcgtaagctatctttcatgtgtcaataaagcttagcaaagctataatcttttatgtttctcaaacgttatcaaaactttccctgcaattttgttttcagtttataaaacaacacataaagcttatgaatacacagttatcaactaactaaactgatagtaagattaggaaaaaagaaaacaaaaggtctacacaagtaattactcacattcccaacttgcaaagcctttttttgaagattaccactatgtactgaactaataagactctcaagttatatatggatcacacctgaaaataaacattggttataatcttaagaaatctaaaacatagcatttacatatatcttataactagaagagttgtatgtatagcgttctcaacttctccaatgaaagaaaagattggtatgagtaggaggagaaatatataaaaaagcttctagagaagattgaatgatctctttgctgtgaaattttcaagtgcgaactcatcagttctgttcactgcttctttgaaattgcttgtgctcttccttgcattttccttcttagaatagtataagccaaaagggaatggtgggactttatgcaccttatttttcgttggtgacatatttttccacaaaagtctttgtaagtcggccgagtcaacgttttatgacatatacgcaattacgcatacaaaattgtcacaagtctctttggttcattattgcgttacatatcttctttagaaatattcttggtccctctctctctaccacacagattgattatcccttctaatcatcccctctacaattcttgctgtatgcttcagattccatcaagaaaaccaagaaactgattttcttttttttcaacttcggtaaaaaaaaaggcagctttttgaaaatgaaatttcaatcgtttttgaaagaaattaagaaatgtgaatcacaagagacttattgaagaagaaagagttttgactgcaatatttttatatctttcaacttggttataaagatacaactctaactctatttatatgccaagcgcaacctcaaaaccctaatgaacctctacacgtgtttggaggacattgaatctattctttgttgcaaggtggtgagctgtacgcggtgccaaagtggtcctgctcgtcgtacggtttggctctgcaatatgaacgttgagaggttgagctaaagcaggctttgtcttcatctttgactggtctccgaaactcatatcattgggcttgggctttgttgttgtcttgggctcagcctgactaataagaaacagagagggaagaggaaaagagatgaactttcgaatccagcaagaaagaagatccgagttcagaacctgagccaaatccaagtaccatcatcacccccaccttctccttcatcacttccatcctctttgtctctttcatcagctccatcttcttcgtctcataactggacacacgatgtctttccaagcttccgcggggaagatgtccgcatagggtttctaagccacattcaaaaggagtttaaaagaaaaggaatcacaccattcatcgacaatgagatcaggagaggagaatccatcggtccagaactcatacgggccattagaggatctaaaatcgccatcatcttgctctcgaggaactatgcttcttcaaagtggtgccttgacgagttggtggagattatgaagtgcaaagaagagttaggccaaaccgtaatccccgttttctataaagtagatccatctgatgtaaagaagctgagaggatattttgggaaagttttcgaaaaaacttgcgagggtaaaagtaaggaggatactgagaaatggagacatgctttggagaaggtggccacaattgctggttacgattcaagaacctggtttgtccttttctatttctttttgtttctaatctttcaaactcatacaaatctaactcaaaaattccaaatgatcacagttttttcctcttaatttttacttgctctttatatgcatcaatatataatatcaatgttaatctcaaaatttaacaaaaggtgttagttccaaaaattcaaaaacttgtgaaaacatataccattctctaaattttgaacatgtttggttatttaatgaagagatattgggagcgtgaattcaaatagagttgcttaatttttaatttaagagttttgtgtatttaacatcatccaaaaacatgcaatgtgacattcttgagaattgcacacagcatatttccagccaaacaattcttatttggaaaccgtttgagccgatgtatgttatttcctctgtttagggataatgaagcggccatgattgaggaaatagccacagatgtttcaaacaagctgattagttctgttccatcaagtgatttcaacagcttagttgggatgagagctcacatgaaaagtatggaactgctcttacgcttggattccaatgaagtgaggatgatagggatttggggtccttctggaattggtaagagcaccatcgccagatctctctttagccaacactctcctgactttcaacttagcgtcttcatggagaatatcaaaagagagtatccaagaccttgttttgatagatacagcgcacaactgcaattacaaaagaagttcttgtctctaatactcaatcagaatgatgtcgctatccatcacttaggagttgcacaagaccggctaaaaaataagaaagtgttagttgttctcgatgacgtggatcactcagcgcaactagatgccttggcgaaagtaccctcatggtttggtcctggaagtaggattatcgtcacgacgcaagataagaaaattttgaatgcgcatcggatcaatcatatttacgaggttggttttccacatgatgatgaagctcttgaaatcttctgcataaatgcttttggtcaaaaatccccatatgatggttttcgaaaccttgctcgggaagttacaaggcttgtgggtaaactccctttgggactaagtgttatgggatcttatttcaaaggcttgtccaaggaggtgtgggaacgtgagttaccaaggttaagaactagacttgacggtgaaacagaaagtattttaaagttcagctatgatgcgttatgcgatgaagatcaagctttgtttcttcacatagcctgctttttcaacggtgaacggattgacaaagtagaagagtttcttgcagagaaatttgttgctgtggaaggtcgtcttcgtgttttagctgagaaatctctcatatccgtagactcagaaggatatataaggatgcatgatttgctagcacgtttgggtagggaaattgttcgtaaacaatctcctaacgaacctgggcagcgtcagtttttggttgatgatggagatatacgccaagtactacgtgatgatacacttgtaagttttcacgttggttgttctcagcatttctcctagaaaatgttataacaaatcatgtttgcttatgtatctttgggttctttttcagggtagtcgaagtgttataggcataaaatttgagttggggaagaaggagttgaagataagtgatggagcctttgaaagaatgtccaatgtccaattcttaagacttgatagtgatttatttgaccatattctactcgtacgaacaaatagccaatacatattagaaagtgtgaactgtctacctcgagaagttagattactgcattggagcacattcccgatgacatgtctgccttctgattttaatccagagctcctaatggaaataaaaatgagatgtagcaaccttgagaaattgtgggaaggaaataaagtaagtacaatatatatacatatttcaaaattatatgctttagctgaggatcgtctttttcttaatgatgcattttgtttatctgtacagacgattagaaatctcaagtggatggatttgtcttattcaaaatatctaaaggagcttccaaatctttcaactgccacaaatctccgagaactggatctcgatatttgctcaagtcttgtggagcttccttcttctattgggaatttgactaatctcaagaaattgaatctcgagctgtgctcaagccttatggaactcccctcttctattggtaacatgactaatcttgagaatttgaatctttctggatgctctagccttgtggagctcccctcttctattggaaatatgaccaatctcaaggaattggatctcagtgaatgctcaagccttgtggagctcacctttgggaatatgaccaatctcaaggatttggatctcaatggatgctcaagccttgtggagatttcctcttctattgggaatatgactaatctcgtgaaattggatctctccagatgctcaagccttgaggaactcccttcttctattgggaatatgactaatcttgagaatttgaatctctccggatgctcaaagctaaaagcccttccgatcaacattaacatgaaatctcttgatgagcttgatctcacatactgctcctcgatgaaaaggtttcctgagatttccacaaacattagcgttctaaagattgatggaactgctataaaagaaattcctgcatcaatcagttcatggtctcgtcttgataggttacatgtgtcatacagtgaaaacctcgggagatcccgacatgtttttgaccgcatcaggaagctggacttgaacgacacaggattacaagaaattgctccatgggtcaaggaaatgtcttgtctagagacactagtaatccacggatgctcaaatctacaaaagctccgctcttccattggaaatttgactaatcttgagaatttggatctcaaaggatgctcaagccttgtggagctcccctcttctattgggaatcttcataatttgaagcagttgaatctcggaaattgctcaaagctaatgtcccttccagtgaacatcaacatgaaatctcttgatgagcttcatctcagggactgctcgtcattgaaaagctttccggagatttccacaaacattggagttctaaagctcaacggaactgctattgaagaaattcctcaaccaatcaggtcatggtctcgtcttgaaaggttacatatgtcatacagtgaaaacctcgggaaatcccagcatgcttttgacctcatcacagagcttcacttgagcgacacacgaatacaagaagttgctccatgggtcaaggaaatgtctcgtctacataaacttgtaacgaagggatgcacaaagctggtttcgctcccacagcttccacattcattagaattcatgcatgtagaaaactgcgagtccctggagagactagattgctctttttacaggacaaagttgagtgatctttgctttgttaactgcctcaaactgaatcgagaagcagtagaccttattctcaagacatcgacaaaaggatgggtgatctttcccggagaaacggtgcctgcatatttcagttacagagccacggggagttcagtgtcaatgaaactggatggattcgatacacgttttcctacatccttgagatttaaagcttgcatcttgcttgttactaaccctgacgacgttgagcctgctgcttggtacaggtcggatatatcttattgcatcaatggcaaactgagggatgtcggtgttttactattattttctcatatatgggacccactattaatgtatagagtctggtttagtccggtttagttgaataggtattatgattctcatttggtttacaggattagtcaagtgtatatatatcttgtaccaaactcattttgtcagttgagaaataagagatcattttacatacgattcatctttactcatgatctctattatggtatcagagctcttttagctcgtttttcttcgattcctcttcgttttcttcccaatcatcctccgatcgttggttcttcactctattcctcaatttctgatcgtatcgagctcagatctggtgaaaaatggtgaagacagggggaaagatgagaactcgaagcgcgatatcggaagacgaagattccacgagcagatttcgtcaggatttccagaatctcgatgaacctctcactgcgatgacgaaaactcaacagaatcgccaatcgatgactccagggggaaacggatctatgcgaaagccatcagaagcgtacgatagcatccacagtccgttcttcctccattctgcggatcatccaggtttgacgattgttgttcatactcttgacgggttcaaactacaacagctggtcgattgcgatgaaaataagcttagatgcgaagaataagcttagctttgttgatggatcgcttcctaggccgttagtggatgataactcgttcaagatatggagtcgttgcaatagtatggtgaaatcgtggattctgaatgtcgtgagtaaaggagatctatgatagtatcttgtattatcaggatgctactgagatgtgggatgatctgtttcgaagattcaaagtgtgtaatctgccacggaaatatcaattggagcaagcggtgatgactctcaaacaaggagatcttgatttgtcaacctatttcacaaagaagaagacactgtgggaacaactagctaacacgaagtctagcacggtgaagcgttgtgattgtgatcaagtcaaagaacttctggaggaagcagagacaagccgtatcatccaattcttgatgggattgaatgataatttcaataacattcgtggtcagattcttaacatgaagccaaggccgggattgaatgacatctacaatatgcttgatcaagacgagagccagcgagtagtaggaggaccttcggtcatgaagcctactccatcagcgtttcagaatcaagcacagtttgcagatcagaatcaaaatccggttctcatggcacatggtggtggttatcagaaaccaaaatgctcacattgcttccgcattggacatacagttgacaagtgctacaaagtccatggttatccaccaggacatccacgtgcaaagaagagtaacacgattggtaatactaacttggctgcaacgatgactgtgtcacagaaccagaatgagcaaggttttgatgagttaagcagtaatatgtctaaggaacagctccagtagatgattgcgttcttcagttccaagctccattccccgagtgttgctccatgctcagataaatcagtagcctcaacttcaacttctgtaccagttatatctcaaatttctggtactttcctcactctctataataactcttactatgacatgctgacttgttctgtatctaaagaaactgagctgtctcttagtgcttggatcatagattccggggctagtcaccatgttactcatactagggaattatatagagagtatagaagtctagaaaacaccttcgttacccttcctaatggttatacagttaagatagcagggactggttacattcaactcactgatgctttgtctctccataatgtgttacatattcctgaattcaaattcaatttgctgagtgttagtgttctaactaagactttacattctcaagtttgtttcacttctgatgcttgttttattcaggctcttactcaggagttgatgattggtcaaggtagtcaagtcgcaaatttatatgtcttagatcaagagaattcgcgtattaatctcattgctccaggtaacttctctgtttgctccaacctggtagttgattctctgacttggcatagaagattaggacatccgtctatgaagaaaattgaatctctttccacaattttaaatttccctaaggataaacatgttaaacattctgatgcgtgtcatgtttgtcatttgtctaaacaaaagcggttaccctttataaaccgaaatcatctttgtaataaaccatttgagttaatacatatagatacttggggtccattctctgttcctactgttgatggttacaagtatttcttaacaattgttgatgattttagtagagctacttgggtttacttgcttagacagaaatctgatgtgttacatgtttttccgggttttattcaaatgattgagaatcaatttcacatgaatgtgagtgcaattagatctgataatgcaaatgaattgaatttcactaatctatatttgaataaaggaataaaagcttaccattcttgccctgaaactcctgaacaaaattctgtggttgagagaaagcatcagcacctgttaaatgtagctagagcccttatgttccagtcaggtatgcctttagaatattggggggattgtgtattaacagcagtttttcttattaatcgtttaccatctcctgtcttagagaataagacaccctatgagcgtttaacctctcaagttcctgattatcattcccttaagacttttggttgcctttgttatgtgtctacgtcacccaagtctaagaataagtttgaacctagagctaaagcctgtgtgttcctaggataccctgctggttacaaaggatacaagcttttagatatagagactcgctcagtttcaatttctaggcatgtcattttctatgaggatactttcccttttgcttcttcaaacattcctgaagatgtcaaaagtttctttcctcatcttcatttccctgcacaaactgatgtattgccttctatgcaaacatcttctgatactcatcatccttgtgatgagtcatcttctccggcttttgttccctctgaaccgaagtctgctaggcaacgcaaaccgccttctcatttgcaggattttcattgctatactactaataacactactcctataaatacaactccatatccattacaaaattttatttcttattcttatttagctgagccttttagtgctttcataaatgccattacttctttcaaaattcctcaaagactttccgaggctcttgaagataaagtatggcgtgactcgatgggattggaaattggagcttttgatcgtactgaaacgtggagtgttgttgaattgcctccaggcaaaactgctataggttgtaagtggcttcatactgtgaaatttaaccctgatggtacagttgagagagttaaatcgcggcttgtaggtaaaggttatacacaacaggagggcattgattttctagatacattttctcctgtcgctaagatgactacagtgcgtttgatctttgctttagctgctaaattaaagtggcatttacatcaattggatatttccaatgctttcctcaatggtgatttggatgaagaattatacatgaagcttccaccggggtatgctgagattaagggggaacaaatctcaccaactgcagtgtgcaaattgcataagtctatctatggcttaaagcaagcgtcgaggcagtggtttttaaaattctctgcaactctgttgggttttggctttgagcaatgtcatggagatcacactctgttcatcaaagaacttgcggggcatttcttgatagtcagtgtgtatgtagatgacatactcattgcgagtacatctctggagggagtgagtgagttgatcagcagtttaagtgcagttttcaagttacgtgatttgggtatacctaagtattttcttggcattgaaattgcaagaacagctgaaggcatttatttatgccagaggaagtatgtgttggatctattggaggcatcgggattttctgattgtaaaccttcttccatacctatggaaccgaatcagaaattatctaaggaggatggagttttgattgaagatgttaagcagtatcgaagattggttgggaagcttcagtatcttacaaataccagacctgacatcgcgtttgcagtatccaagcttgctcagtattcttgtgctccaacagatattcatttgaaggctgttcataaggtgttgaggtatctcaaaggtactattggacagggtgtgttctatggagtggaggataattttgatttgcgagggttctctgattctgactggggaggttgtcctgatgataggagatcagtcacaggttatgcgatgtttctgggcagttctttggtatcttggagatcacagaagcaagacatagtatcgatgagtacagctgaggcggagtatcgtgctatgagcgtggctactaaggagattatgtggtattgtggtgttctcaagtctgtccgagtgccgttttctcctccagcttacttatattgcgataacactgctgctctttacattgctactaattctgtttttcatgagcgaaccaagcatgtggagtttgattgccataaggtgagagagtgcatcgtccgtggcatcttgaagacaatgtatgttcgtaccgacaatcaactggccgatgtgttgactaaggcattgtacccagctccatttcgagacattatcagcaagatgggtgtttttaacctttatgcaccttcatcttgagggggaatattaatgtatagagtctggtttagtccggtttagttgaataggtattatgattctcatttggtttacaggattagtcaagtgtatatatatcttgtaccaaactcattttgtcagttgagaaataagagatcattttacatacgattcatctttactcatgatctctattacccactctgtccacgttctgagcatctggtcgtaatcgaatttgaagaaactgtgacttcccccgaattagtcttcgagttcaggttcaaaaaagaaaactgggagattaaagaatgcggactacgtcctctagaaagcttagctctctcatgttgatggaagctgaagaaacattttgcagtatgtgtgtaagattatcattgtgtgttatgtttggtttgagtaaaatgtaacggcaactatatttatgtacgcatgtgcataaaatatagtaatgcattccgttgtttatcttaaatatcaaggaaacaatttttattagtaatctttgtttcctactcgttgagtg

(19) United States
(12) Patent Application Publication
(10) Pub. No.: US 2013/0254929 A1
(43) Pub. Date: Sep. 26, 2013

(54) METHOD FOR PRODUCING CRUCIFEROUS PLANT RESISTANT TO CLUBROOT

(71) Applicants: Satoru Matsumoto, Tsu-Shi (JP); Katsunori Hatakeyama, Tsu-Shi (JP); Nobuko Fukino, Tsu-Shi (JP)

(72) Inventors: Satoru Matsumoto, Tsu-Shi (JP); Katsunori Hatakeyama, Tsu-Shi (JP); Nobuko Fukino, Tsu-Shi (JP)

(73) Assignee: Incorporated Administrative Agency National Agriculture and Food Research Organization, Ibaraki (JP)

(21) Appl. No.: 13/825,783

(22) PCT Filed: Sep. 22, 2011
(86) PCT No.: PCT/JP2011/071554

§ 371 Date: Jun. 3, 2013

(30) Foreign Application Priority Data
Sep. 22, 2010 (JP) 2010-211689

(51) Int. Cl. C12N 15/82

(52) U.S. Cl. CPC C12N 15/8282
USPC 800/265; 536/23.6; 435/320.1; 435/419; 800/301; 435/6.12; 800/279; 536/24.33; 536/24.3; 435/6.11

(57) ABSTRACT

Successfully produced are cruciferous plants resistant to clubroot by introducing the clubroot resistance gene (Crr1) isolated by map-based cloning into cruciferous plants and expressing the gene.

### TECHNICAL FIELD

The present invention relates to clubroot resistance genes and methods for producing cruciferous plants that are resistant to clubroot by using these genes.

### BACKGROUND ART

Clubroot is caused by Plasmodiophora brassicae. It is a soil-borne disease and is difficult to prevent; and it affects cruciferous vegetables such as Chinese cabbage (Hakusai, Brassica rapa L. Pekinensis group), turnip, Nabana (Brassica napus L., Brassica rapa L. Oleifera Group), Nozawa-na (Brassica rapa L. Hakabura Group), Tsukena (Brassica rapa L. Perviridis Group), cabbage, and broccoli in Japan, and rapeseeds abroad. Since the roots of the diseased lines enlarge in the form of a club, they pose problems in nutrient and water absorption, and cause significant delay in growth, or in some cases, cause plant death. Once this disease occurs, a large number of resting spores are released into the soil from the infected lines. Since the resting spores exist in the soil for a long period of time and maintain their ability to germinate, their effects cannot be expected to be reduced by crop rotation; and in a continuously cropped field, the fungus density increases year after year, and cultural control is difficult. Therefore, it becomes necessary to cultivate dependently on chemosynthetic agrochemicals or change crops to vegetables other than cruciferous plants.

Clubroot resistance genes were not found in the genetic resource of headed Chinese cabbage belonging to Brassica rapa; and diligent research by Yoshikawa at the National Research Institute of Vegetables, Ornamental Plants and Tea proved that European fodder turnips such as Siloga, Gerlia, Millan white, and 77b are promising resistant materials (Non-Patent Document 1). Furthermore, a number of resistance genes have been found in these European fodder turnips. The pathogenicity of clubroot fungi, plasmodiophora brassicae, is diverse, and as a method to identify them, the European Clubroot Differential (ECD) method, the Williams method (Non-Patent Document 2), and such have been proposed. In Japan, indicators for assessing the pathogenicity of domestically emerged strains (Non-Patent Document 3), and classification methods using readily available cultivars (Non-Patent Documents 4 and 5) have been reported.

With regard to the cultivation of resistant cultivars of Chinese cabbage (Clubroot Resistance; CR cultivars), a major resistance gene was found in Brassica rapa, and by devising high-precision resistance assays and establishing methods capable of distinguishing differences in pathogenicity, six parental lines of Chinese cabbage were reported from the National Research Institute of Vegetables, Ornamental Plants and Tea, and clubroot-resistant cultivars of Chinese cabbage were cultivated in private seed companies. On the other hand, resistance breeding could not catch up with the speed of race differentiation and spreading of clubroot fungi, so that the resistance of the clubroot-resistant cultivars was lost and reports of infected cases have increased. Among the clubroot fungal isolates, isolates that damage many resistant cultivars and have a wide host range became noticeable. Therefore, chemosynthetic agrochemicals and materials promoting decrease of clubroot are being used even when cultivating resistant cultivars. Use of chemosynthetic agrochemicals such as Nebijin or Furonsaido imposes great burden on the farmers in terms of cost and labor. What is desired is the development of CR cultivars that can cope with a wide range of clubroot fungi races, or specifically, accumulation of a plurality of the introduced resistance genes. However, there are problems in terms of precision and efficiency by conventional selection according to phenotypes such as clubroot resistance. Furthermore, there are few major genes in B. oleracea to which cabbage and broccoli belong; and since a number of resistance genes need to be brought together to exert resistance, breeding and cultivation of resistant cultivars are considered to be extremely difficult work.

Cultivating resistant cultivars that can be grown without the use of chemosynthetic agrochemicals is required at the social level. However, as described above, there are many technical issues that have to be solved to accomplish this objective.

To date there have been no articles reporting the isolation of resistance genes for clubroot in cruciferous plants. Meanwhile, a research group at Kyoto Prefectural University is in progress of isolating Crr3 derived from a European fodder turnip “Milan White”, and at present they are known to be confirming the work by transformation. The only case in which clubroot resistance was conferred by genetic recombination was generation of a resistant individual by linking an antifungal peptide (Scarvaecin derived from Taiwanese unicorn beetle) gene to a nitrilase promoter and introducing it into broccoli (Patent Document 1). The current method for developing resistant cultivars is mainly through breeding by crossing and selection.

In the development of markers for efficient selective breeding in B. rapa, many gene loci such as RA1275 (Non-Patent Document 6), Crr1, Crr2 (Non-Patent Document 7), Crr3 (Non-Patent Documents 8 and 9), Crr4 (Non-Patent Document 7), CRa (Non-Patent Documents 10 and 11), CRb (Non-Patent Document 12), CRc, and CRk (Non-Patent Document 13) have been reported. Outside of the National Institute of Vegetable and Tea Science, patent applications and such have been submitted for markers linked to rutabaga-derived resistance (Patent Document 2).

At the National Institute of Vegetable and Tea Science, as DNA markers that are linked to Crr1 and Crr2, BRMS-173 and BRMS-088 (for Crr1 above), and BRMS-096 and BRMS-100 (for Crr2 above) were developed, and a patent has been acquired for their use (Patent Document 3). In the development of markers related to B. oleracea, gene loci that have a relatively small contribution rate as compared to major genes have been reported; and while QTL such as pb-Anju-01 have been reported recently by Nagaoka et al. (2010) (Non-Patent Document 14), their use in actual breeding is presumed to be low.

Furthermore, there is one past example of cultivation of plants that were conferred clubroot resistance using genetic recombination techniques. This involved operably linking a promoter of a gene specifically expressed during clubroot fungal infection with an antifungal peptide, introducing it into broccoli, and then confirming the resistance. Use of the promoter has been filed for patent (Patent Document 1). However, the disease index of the plant obtained by introducing this set of promoter and gene was 1.36 to 1.50. Furthermore, the very clubroot resistance gene itself has not been used to confer resistance by genetic recombination techniques.

While there are many reports on markers for selection of clubroot resistance relating to Chinese cabbages as described above, the extent of their use in actual breeding is unclear. Crr1 is known to be positioned approximately at the center between BRMS-173 and BRMS-088 which are 4-cM away from each other. Specifically, since each of the markers of BRMS-173 and BRMS-088, which are linked to Crr1, are both approximately 2-cM away from Crr1, recombination has been confirmed to take place at a certain constant probability between the marker loci and the resistance gene locus. As in this case, when one cannot eliminate the possibility that recombination is taking place between the marker loci and the resistance gene locus, the progenies obtained after crossing or selection using two markers flanking a gene locus must be subjected to clubroot resistance assay to confirm the transmission of the resistance gene.

While there is information on the narrowed-down candidate Crr1 genes (Non-Patent Documents 15 to 18), none has been proven to be Crr1, and the sequence information has not been revealed either. A number of ORFs are present in the genomic region that has been narrowed down by map-based cloning, but it is unclear as to which of them is the Crr1 resistance gene. Furthermore, there have been attempts to elucidate all of the regions of the expressed genes by RT-PCR; however, the amplified clones were not all the same, and one could not determine which clone was Crr1. All of the amplified clones were compared and examined; and there were unpredictable situations such as the ill-functioning of splicing at the intron regions. Therefore, it was considered impossible to understand which regions correspond to the gene from the disclosed contents alone.

Prior art documents relating to the invention of this application are shown below.

### PRIOR ART DOCUMENTS

### Patent Documents

[Patent Document 1] Japanese Patent Application Kokai Publication No. (JP-A) 2009-178090 (unexamined, published Japanese patent application)
[Patent Document 2] JP-A (Kokai) 2005-176619
[Patent Document 3] Japanese Patent No. 4366494

### Non-Patent Documents

[Non-patent Document 1] Yoshikawa, H., Bull. Natl. Res. Inst. Veg., Ornam. Plants & Tea Japan, (1993) 7:1-465.
[Non-patent Document 2] Williams, P. H., Phytopathology, (1966) 56: 624-626.
[Non-patent Document 3] Kuginuki Y. et al., Eur. J. Plant. Pathol, (1999) 105:327-332.
[Non-patent Document 4] Hatakeyama K. et al., Breed Sci., (2004) 54, 197-201.
[Non-patent Document 5] Hatakeyama K. et al., Horticultural Research (Japan) (2008) 7 (supplementary volume 2) 180.
[Non-patent Document 6] Kuginuki, Y et al., Euphytica, (1997) 98: 149-154.
[Non-patent Document 7] Suwabe, K. et al., Theor. Appl. Genet., (2003) 107: 997-1002.
[Non-patent Document 8] Hirai, M. et al., Theor. Appl. Genet., (2003) 108: 639-643.
[Non-patent Document 9] Saito M. et al., Theor. Appl. Genet., (2009) 114:81-91.
[Non-patent Document 10] Matsumoto E. et al., Euphytica, (1998) 104:79-86.
[Non-patent Document 11] Hayashida N. et al., J. Jpn. Soc. Hortic. Sci., (2008) 77:150-154.
[Non-patent Document 12] Piao, Z. Y et al., Theor. Appl. Genet., (2004) 108:1458-1465.
[Non-patent Document 13] Sakamoto K. et al., Theor. Appl. Genet., (2009) 117:759-767.
[Non-patent Document 14] Nagaoka T., et al., Theor. Appl. Genet., (2010) 120: 1335-1346.
[Non-patent Document 15] Matsumoto, S. and three others, “Molecular genetic analysis of Chinese cabbage clubroot resistance and application to breeding”, [online], KAKEN, internet <URL: http://kaken.nii.ac.jp/en/p/19380008>
[Non-patent Document 16] Matsumoto, S., Kato, T. and five others, “(1) Cultivation of practical clubroot-resistant Chinese cabbage cultivars by marker selection; (2) DNA marker linked to clubroot resistance of the Chinese cabbage F1 cultivar “Akiriso”, [online], internet <URL: http://www.nacos.com/jsb/06/06PDF/117th_{—}611_{—}612.pdf>
[Non-patent Document 17] Matsumoto, S., “Map Based Cloning of the Clubroot Resistance Gene, Crr1, in Brassica rapa L.”, [online], Sep. 9, 2008, Brassica2008-Lillehammer-Norway, CLUBROOT SESSION, internet <URL: http://www.brassica2008.no/clubroot.html>
[Non-patent Document 18] Matsumoto, S., and three others, “Isolation of a candidate gene (crr1) involved in clubroot resistance of Chinese cabbage”, [online], Sep. 28, 2008, Japanese Society for Horticultural Science, Internet <URL: http://www.jshs.jp/modules/tinyd4/index.php?id=7>

### SUMMARY OF THE INVENTION

### Problems to be Solved by the Invention

An objective of the present invention is to provide efficient techniques for producing clubroot-resistant cruciferous plants by isolating clubroot resistance genes, and performing genetic recombination and marker selection.

### Means for Solving the Problems

The present inventors carried out dedicated research to solve the above-mentioned problems.

To promote efficient breeding, there is the method of cultivating by introducing resistance genes using genetic recombination techniques; and for species that can be crossed easily, there is the method of developing resistant cultivars by marker-assisted selection utilizing the genome information around the gene locus. The present inventors provide efficient methods for producing clubroot-resistant cruciferous plants by isolating clubroot resistance genes, and performing genetic recombination and marker selection.

Specifically, first, the present inventors successfully isolated a clubroot resistance gene. By linking the cDNA of the isolated Crr1 to the 35S cauliflower mosaic virus promoter or the lettuce ubiquitin promoter and expressing this in Arabidopsis thaliana, the inventors confirmed strong resistance in clubroot-susceptible Arabidopsis thaliana. By using this method, clubroot resistance may be conferred to species into which genes cannot be introduced by crossing, such as cabbage and broccoli belonging to B. oleracea and rapeseed of B. napus.

Furthermore, the cDNA or genomic DNA of the clubroot resistance gene was linked to an expression-regulatable promoter or a promoter unique to the clubroot resistance gene, and a cassette that enables expression of these genes in plants was produced. This was used to generate transformed cruciferous plants (such as cabbage, broccoli, and rapeseed). This is a novel production method, and there are no examples of using seeds of the obtained transformed plants to cultivate plants that become resistant due to clubroot resistance genes.

Furthermore, because the genomic structure of Crr1 was elucidated, it was revealed that there existed partial insertion or deletion of DNA sequences as compared with individuals without Crr1. These positions can be used as target markers to assess the presence and absence of resistance through DNA polymorphism. In this case, since the information used is a gene region, recombination does not occur between the marker loci and the resistance gene loci. Therefore, extremely accurate selection becomes possible.

Specifically, the present invention relates to the following:

[1] a polynucleotide having clubroot fungus resistance, which is any one of (a) to (d) below:

(a) a polynucleotide encoding a protein comprising the amino acid sequence of SEQ ID NO: 2;

(b) a polynucleotide comprising the coding region of the nucleotide sequence of SEQ ID NO: 1;

(c) a polynucleotide encoding a protein comprising an amino acid sequence with one or more amino acid substitutions, deletions, additions, and/or insertions in the amino acid sequence of SEQ ID NO: 2; and

(d) a polynucleotide that hybridizes under stringent conditions with a complementary strand of the nucleotide sequence of SEQ ID NO: 1;

[2] a vector in which the polynucleotide of [1] is operably linked downstream of a promoter region that enables expression in a plant cell;
[3] a transformed plant cell into which the vector of [2] has been introduced;
[4] a plant having clubroot fungus resistance activity, which is regenerated from the transformed cell of [3];
[5] a plant having clubroot fungus resistance activity, which is a progeny or a clone of the plant of [4];
[6] a propagation material of the plant having clubroot fungus resistance activity of [4] or [5];
[7] a method for assessing clubroot fungus resistance of a test plant or a test propagation medium, which comprises the step of detecting a DNA region comprising the nucleotide sequence of SEQ ID NO: 1 or SEQ ID NO:3, or a partial sequence or surrounding sequence of this nucleotide sequence;
[8] the assessment method of [7], which comprises the following steps of:

(i) preparing a DNA sample from a test plant or a test propagation medium;

(ii) amplifying a DNA region comprising the nucleotide sequence of SEQ ID NO: 1 or SEQ ID NO: 3, or a partial sequence or surrounding sequence of this nucleotide sequence from the DNA sample; and

(iii) comparing the molecular weight or nucleotide sequence of a DNA fragment produced by amplifying the DNA region comprising the nucleotide sequence of SEQ ID NO: 1 or SEQ ID NO: 3, or a partial sequence or surrounding sequence of this nucleotide sequence, from the clubroot fungus-resistant plant or propagation medium with that of the DNA fragment amplified in step (ii);

[9] a method of selecting a plant or seed thereof having a clubroot fungus resistance gene by the assessment method of [7] or [8];
[10] a method for producing a plant or a seed thereof having clubroot fungus resistance activity, which comprises the following steps of:

(i) introducing into a plant cell the vector of [2]; and

(ii) regenerating a plant from the transformed plant cell which has been introduced with a vector in step (i) mentioned above;

[11] a method of conferring clubroot fungus resistance activity to a plant or seed thereof, which comprises the step of expressing the polynucleotide of [1] in a plant cell;
[12] the method of [11], which comprises the step of introducing into a plant cell the polynucleotide of [1] or the vector of [2];
[13] the method of [11], which comprises the following steps of:

(a) crossing a plant comprising the polynucleotide of [1] with another plant; and

(b) selecting a plant comprising the polynucleotide;

[14] the method of any one of [7] to [13], wherein the plant is a cruciferous plant;
[15] a plant or seed thereof which is obtained by the method of any one of [7] to [14];
[16] an artificially produced plant or seed thereof, which comprises the polynucleotide of [1] and has clubroot fungus resistance activity;
[17] the plant or the seed thereof of [15] or [16], wherein the plant is a cruciferous plant;
[18] a primer for detecting clubroot fungus resistance activity of a test plant, which comprises an oligonucleotide having a chain length of at least 15 nucleotides, and specifically hybridizes under stringent conditions with the nucleotide sequence of SEQ ID NO: 1 or the nucleotide sequence of SEQ ID NO: 3; and
[19] a probe for detecting clubroot fungus resistance activity of a test plant, which comprises an oligonucleotide having a chain length of at least 15 nucleotides, and specifically hybridizes under stringent conditions with the nucleotide sequence of SEQ ID NO: 1 or the nucleotide sequence of SEQ ID NO: 3.

### BRIEF DESCRIPTION OF THE DRAWINGS

FIG. 1 depicts a detailed mapping in the neighborhood of the clubroot resistance gene locus Crr1. BSA7 is a marker obtained by synteny analysis with Arabidopsis thaliana, and in an analysis using 1920 segregated populations, it cosegregated with Crr1.

FIG. 2 depicts a BAC clone assembled by chromosome walking from BSA7 near Crr1. A number of BAC clones containing the BSA7 sequence was isolated, and seven clones were used to cover the area between BSA7 and BSA2. The terminal sequences of the obtained BAC were used to develop markers, and the location of Crr1 was refined.

FIG. 3 depicts the marker genotypes of F₂ individuals in which recombination between nearby markers took place by homologous recombination near the Crr1 locus, and the presence of resistance in the F₃ generations of these individuals. The solid black region represents the susceptible variety, Chinese cabbage intermediate mother plant 7 (hereinafter, PL7), and the striped region represents the genome of the resistant line G004. The F₃ individuals 1075, 764, and 572 all show disease susceptibility, and did not carry the resistance gene. Therefore, Crr1 was strongly suggested not to be located at the BSA7-side of B359C3, nor at the BZ2-DraI-side of B359H7, but between B355H7 and B359C3.

FIG. 4 depicts the major (more than 10-bp long) insertion/deletion sequences and the translation regions (solid black regions) of Crr1 determined by comparing the Crr1-containing genomic regions of the resistant line G004 and the susceptible cultivar PL7. “In” indicates the sequence inserted in PL7 and “Del” indicates the sequence deleted in PL7 when compared to G004, and the number at the beginning indicates the number of nucleotides.

FIG. 5 shows the sequence of Crr1 (SEQ ID NO: 1) used in the complementarity test. The start codon and the stop codon are underlined.

FIG. 6 shows the amino acid sequence of Crr1 (SEQ ID NO: 2). Translation into 1224 amino acid residues was estimated.

FIG. 7 depicts the structure of the Crr1 protein estimated from the amino acid sequence.

FIG. 8 shows a photograph indicating the difference in expression of Crr1 determined by RT-PCR in the leaves and roots of the resistant line (R4-8-1) and the susceptible cultivar PL7. R: roots; L: leaves; V-ATP refers to a constitutively expressed gene (positive control).

FIG. 9 depicts the construct used in the complementarity test. Crr1 cDNA: Crr1 cDNA sequence; NPT II: kanamycin-resistance gene; LsUb-Pro: lettuce ubiquitin promoter; and LsUb-Ter: lettuce ubiquitin terminator.

FIG. 10 shows photographs showing the phenotype of Arabidopsis thaliana introduced with lettuce ubiquitin promoter (Up)::Crr1 cDNA. Individuals introduced with Crr1 showed resistance against Ano-01.

FIG. 11 shows a photograph indicating the results of determining the presence of clubroot resistance gene Crr1 by comparing the lengths of the amplified fragments of B359C3. Using each of the DNAs of A, F1, and PL9 as templates, fragments amplified by PCR with B359C3 were fractionated by agarose gel electrophoresis. A: individuals not carrying Crr1; F1: individuals heterozygous for Crr1; PL9: individuals homozygous for Crr1; M: 100 bp ladder DNA size marker.

FIG. 12 depicts the results of comparing the nucleotide sequences of the resistant line G004 and the susceptible cultivar PL7. In the susceptible cultivar PL7, 357 bp have been found to be inserted 60 bp downstream of the start codon, and a stop codon has been found to exist in frame within exon 1. In the figure, the sequence indicated as G004 shows positions 2525 to 2707 of SEQ ID NO: 3. In the figure, the sequence indicated as PL7 shows positions 2602 to 3141 of SEQ ID NO: 13.

FIG. 13-1 shows the result of sequence comparison between the genomic sequence of the resistant line G004 (SEQ ID NO: 3) and the genomic sequence of the susceptible cultivar PL7 (SEQ ID NO: 13).

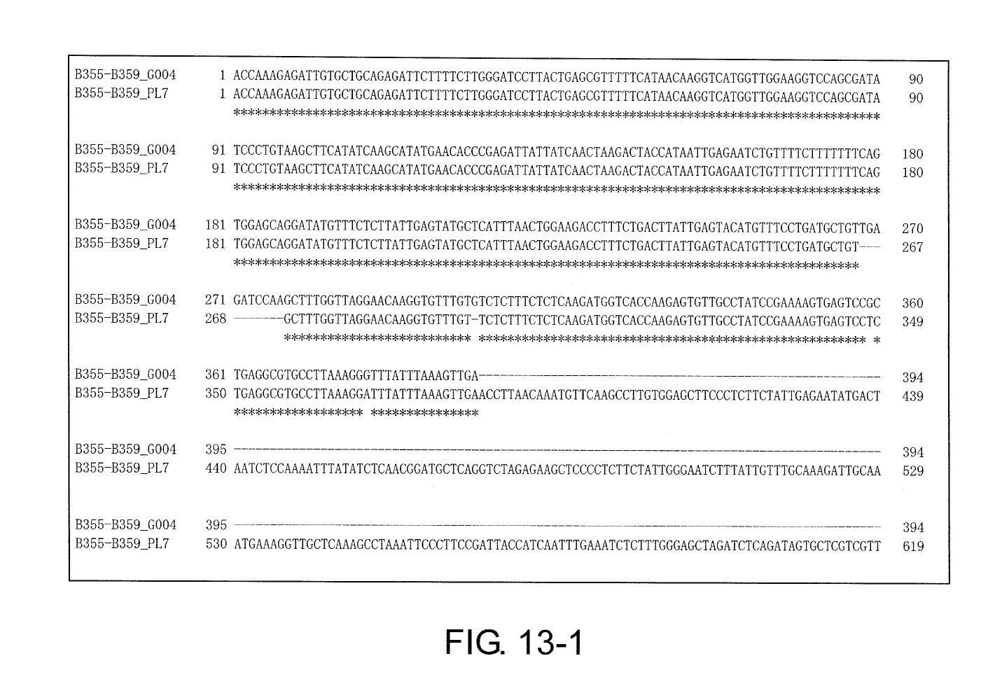

FIG. 13-2 is a continuation of FIG. 13-1.

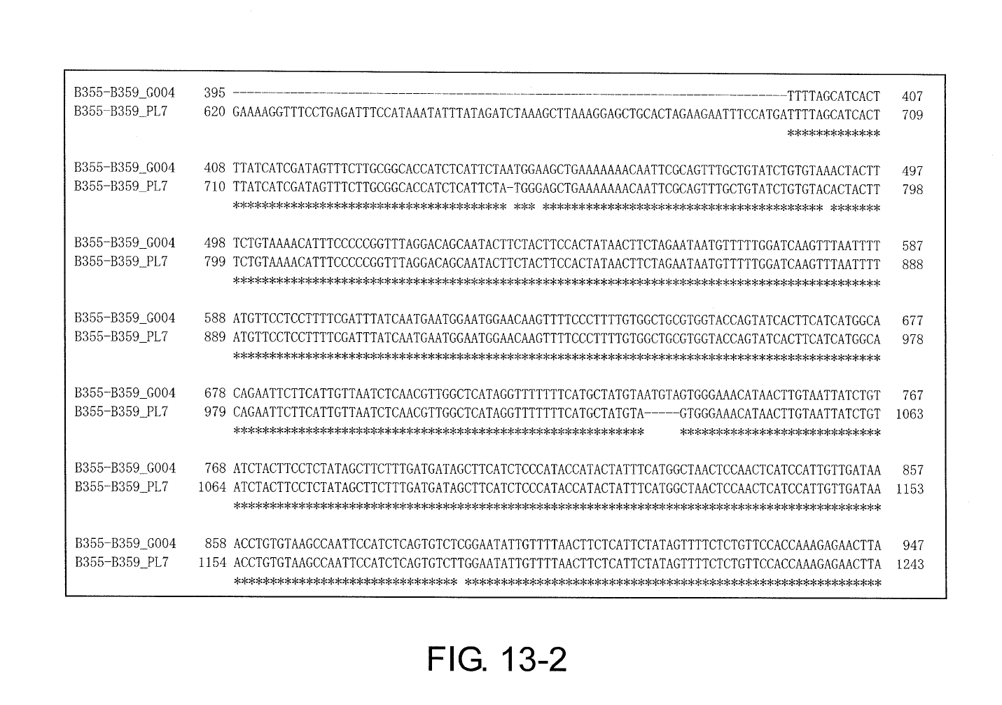

FIG. 13-3 is a continuation of FIG. 13-2.

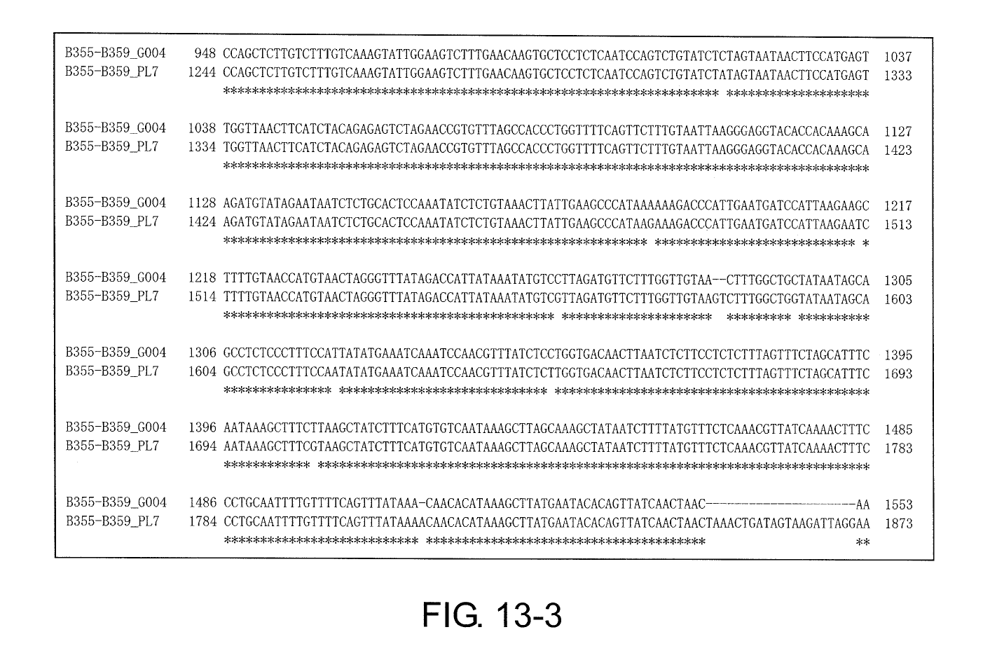

FIG. 13-4 is a continuation of FIG. 13-3.

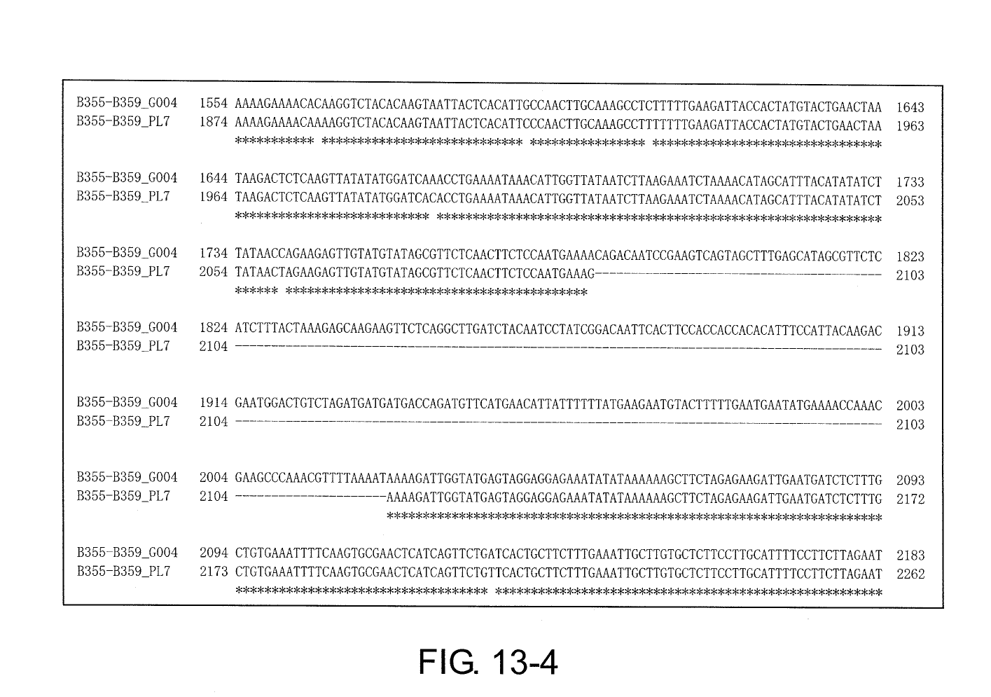

FIG. 13-5 is a continuation of FIG. 13-4.

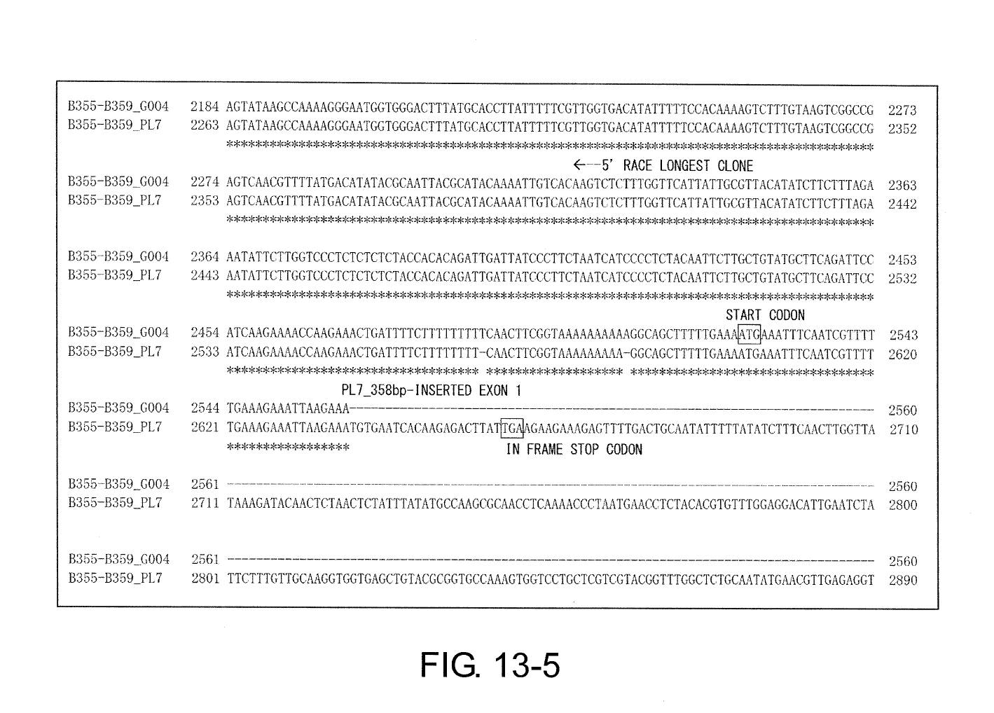

FIG. 13-6 is a continuation of FIG. 13-5.

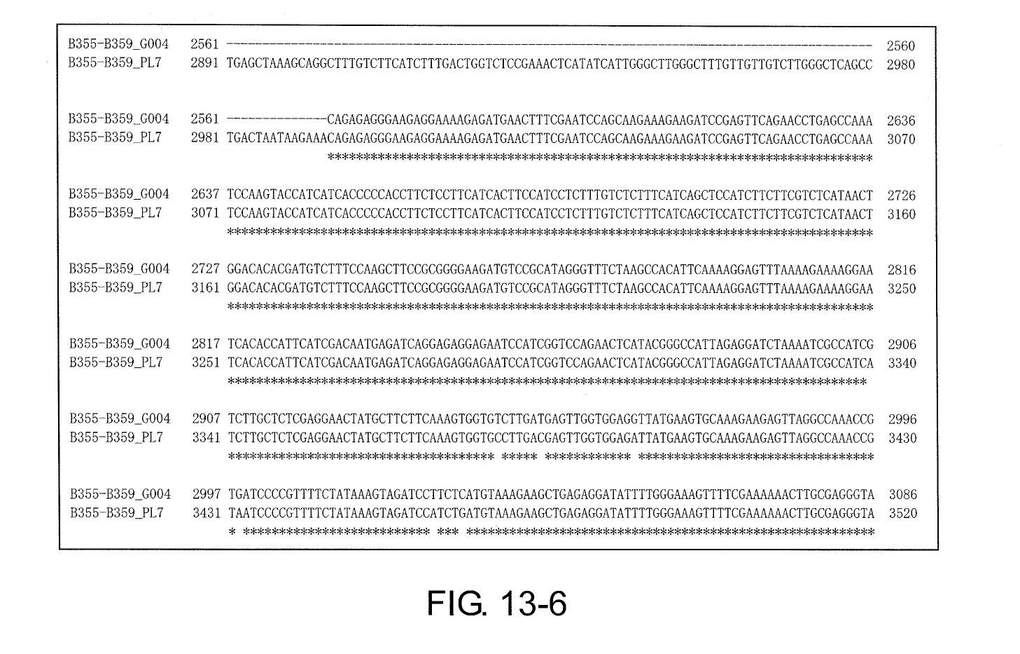

FIG. 13-7 is a continuation of FIG. 13-6.

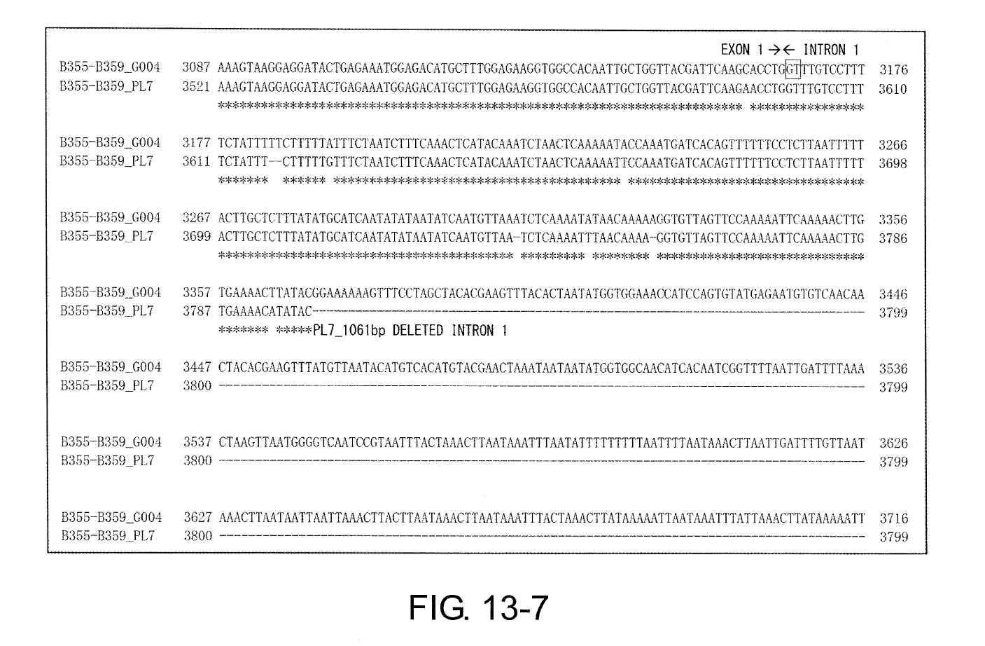

FIG. 13-8 is a continuation of FIG. 13-7.

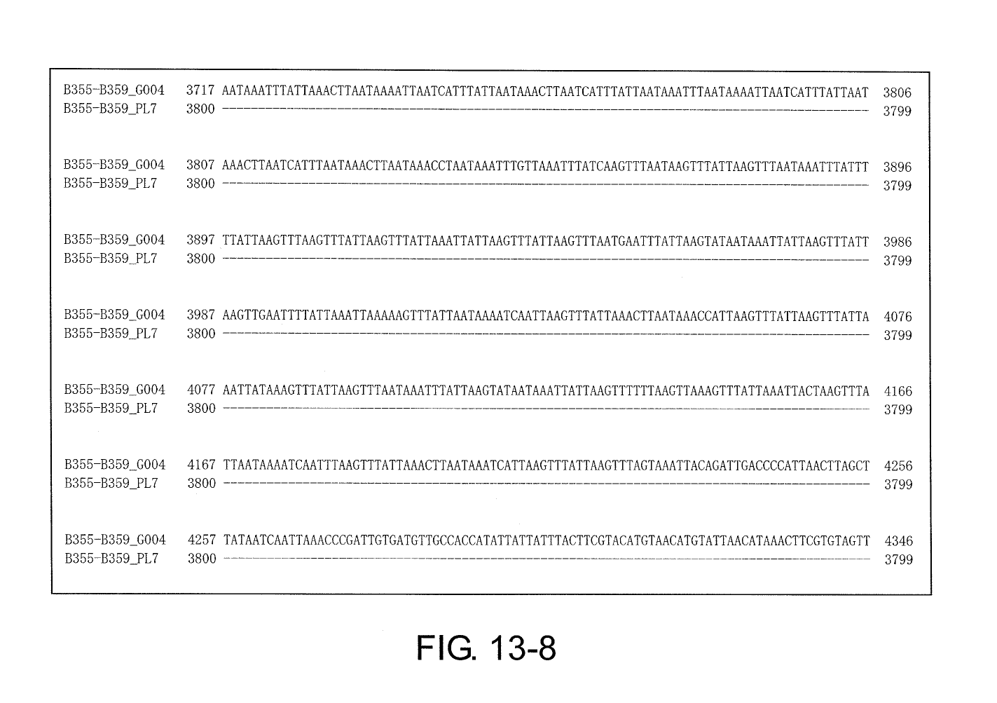

FIG. 13-9 is a continuation of FIG. 13-8.

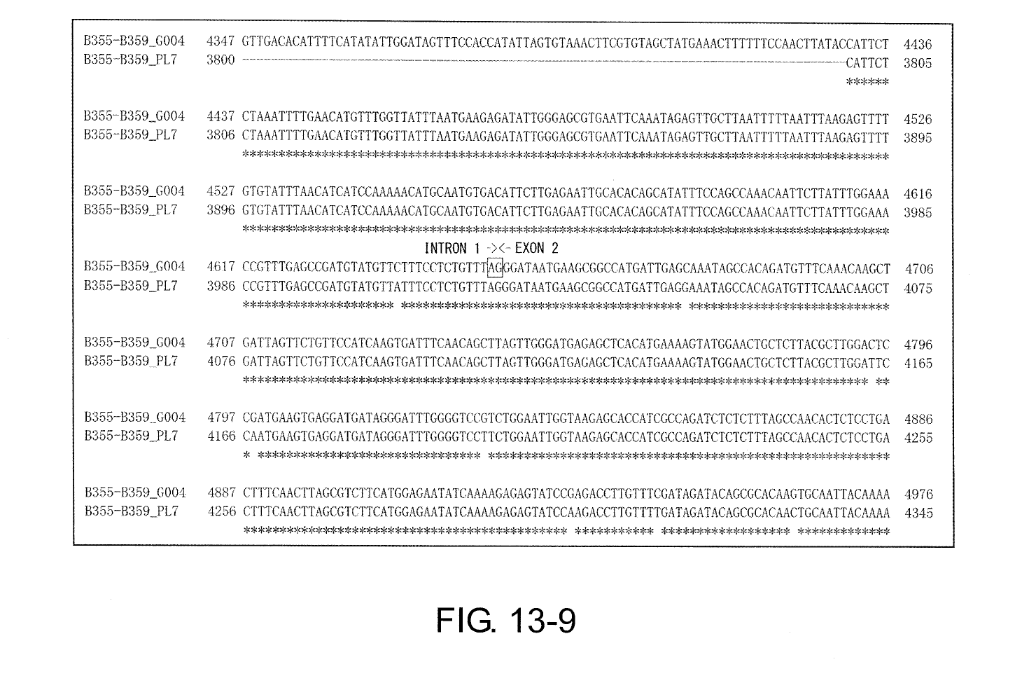

FIG. 13-10 is a continuation of FIG. 13-9.

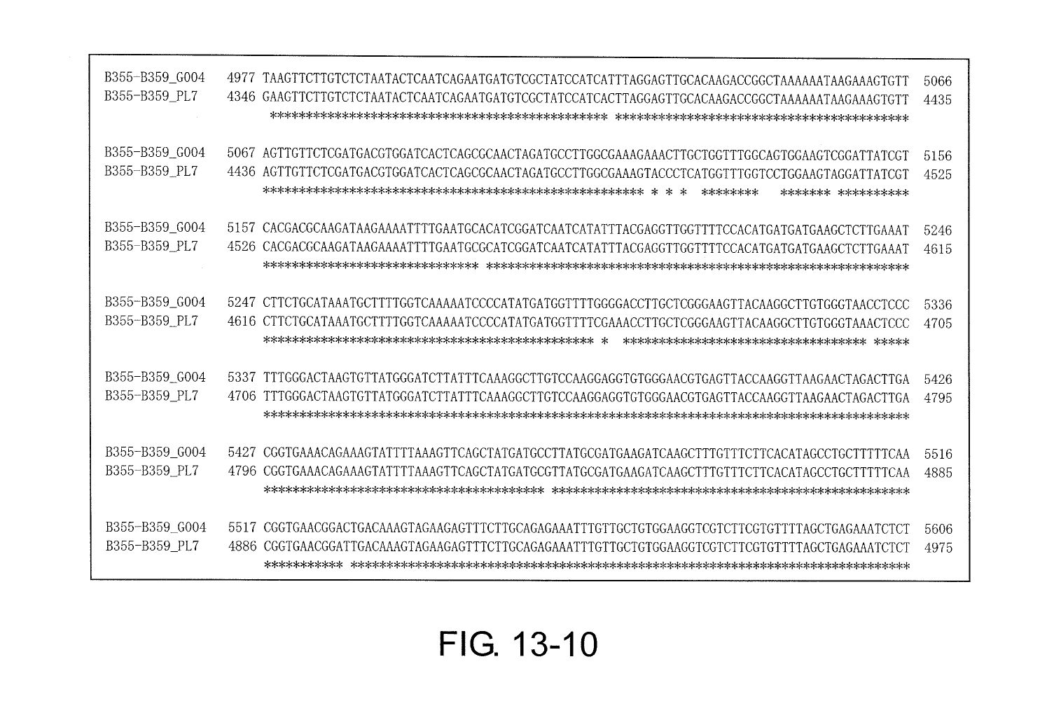

FIG. 13-11 is a continuation of FIG. 13-10.

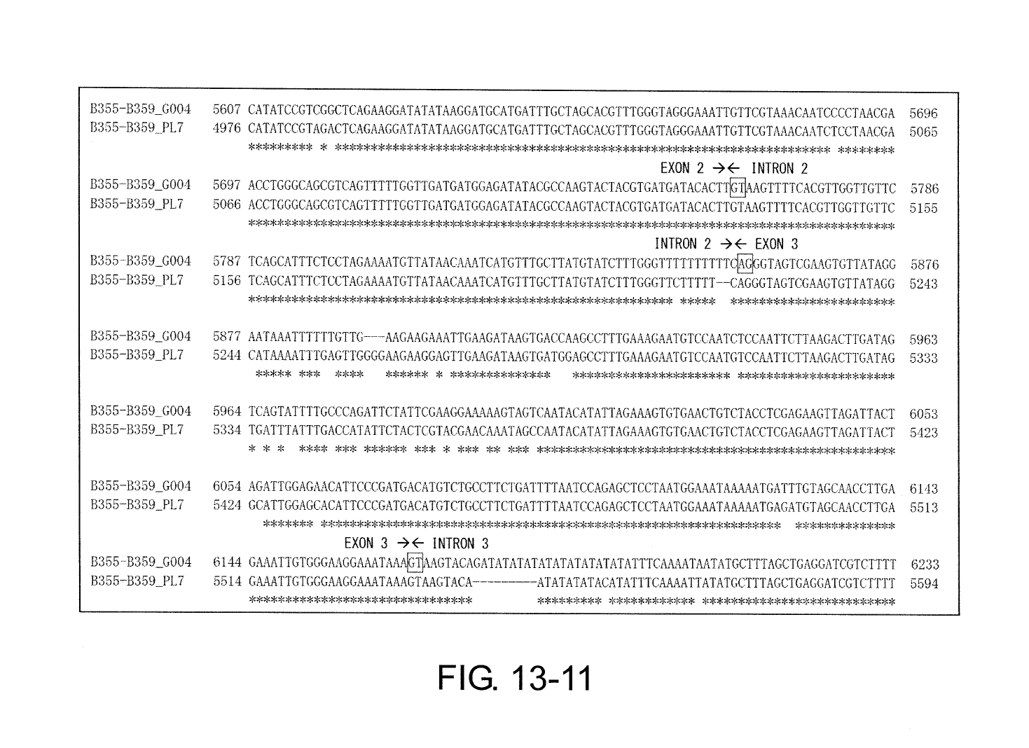

FIG. 13-12 is a continuation of FIG. 13-11.

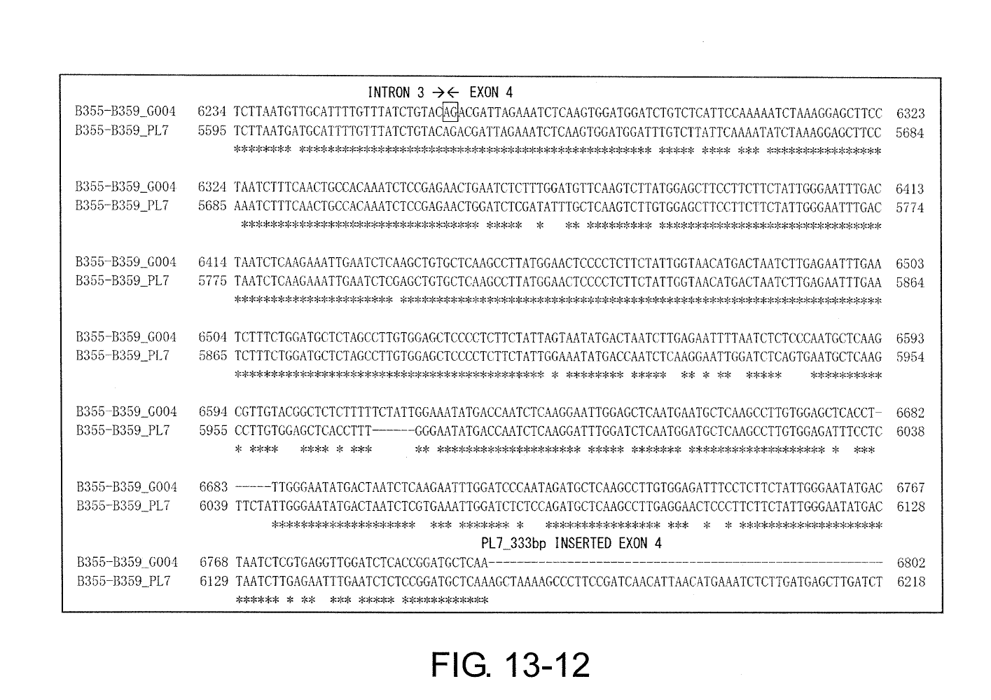

FIG. 13-13 is a continuation of FIG. 13-12.

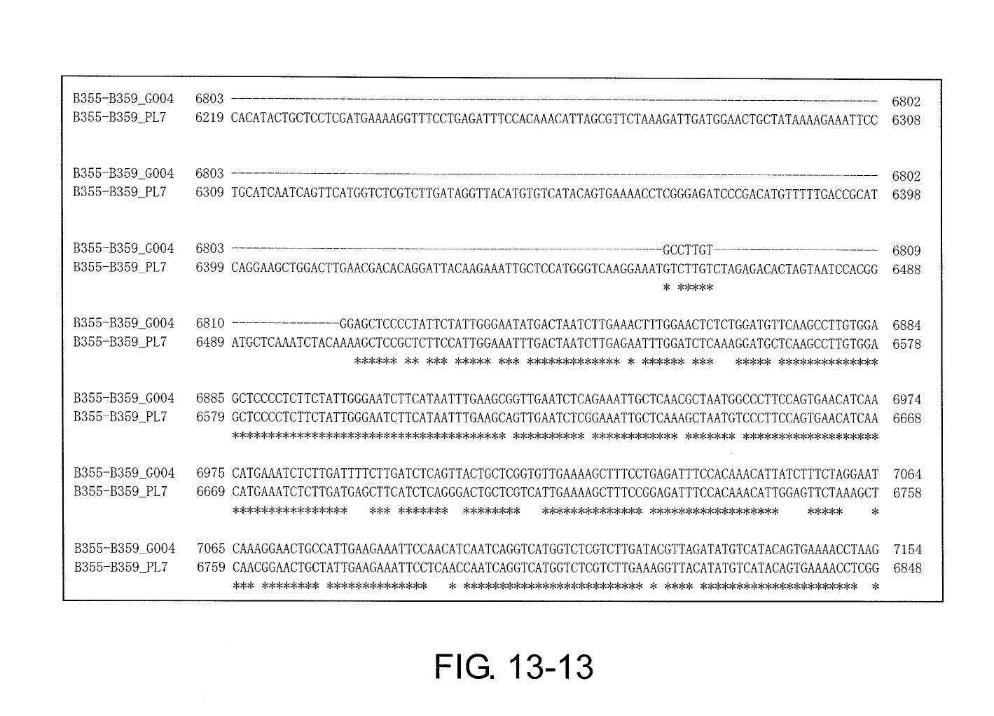

FIG. 13-14 is a continuation of FIG. 13-13.

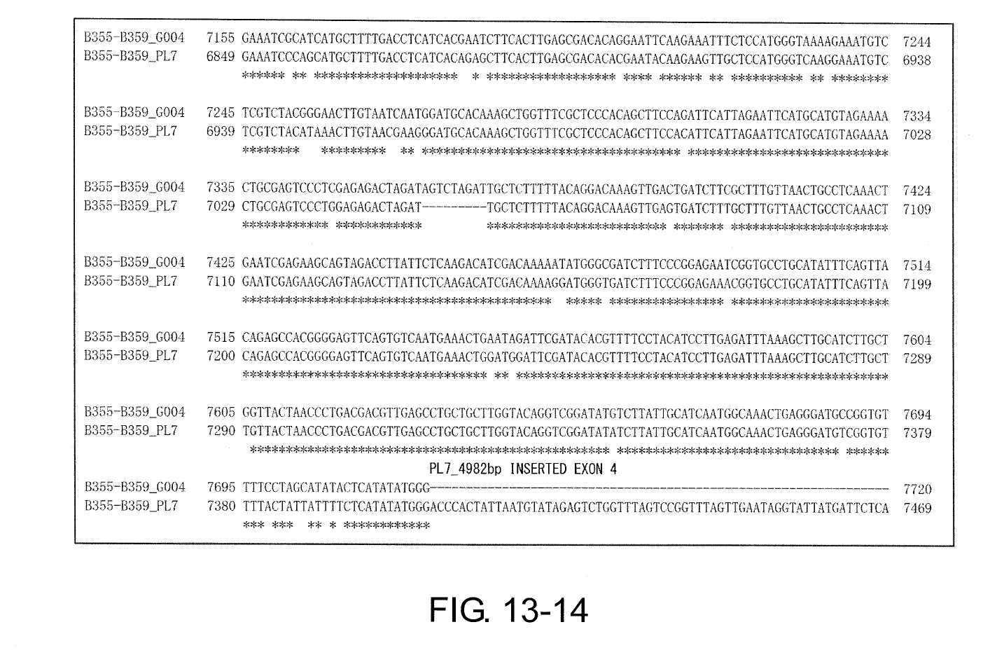

FIG. 13-15 is a continuation of FIG. 13-14.

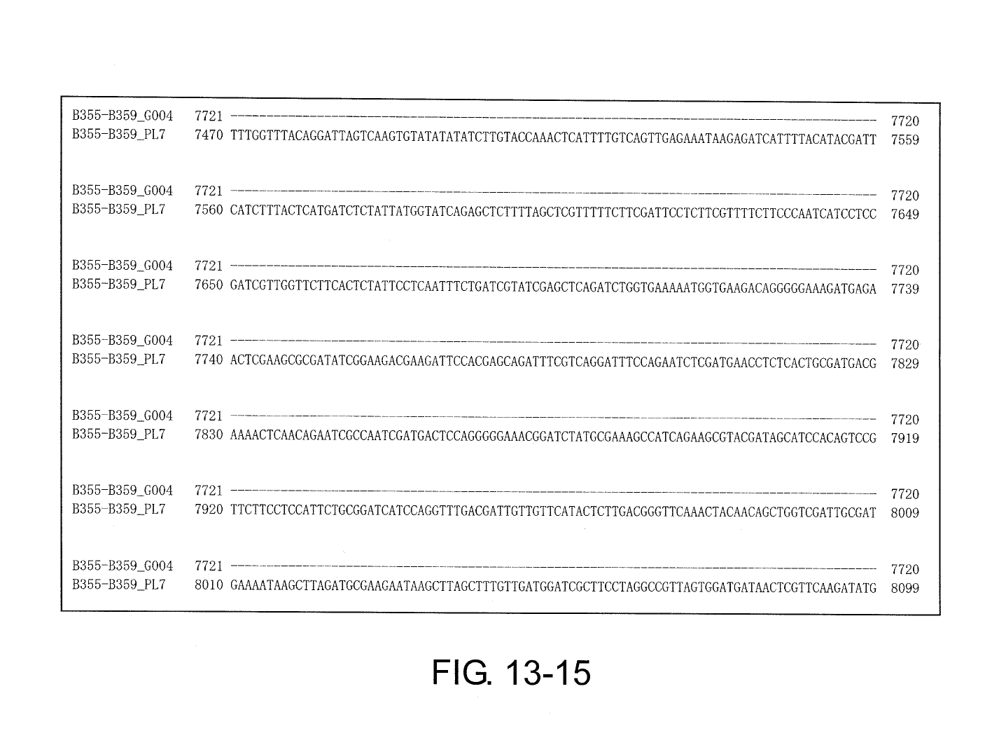

FIG. 13-16 is a continuation of FIG. 13-15.

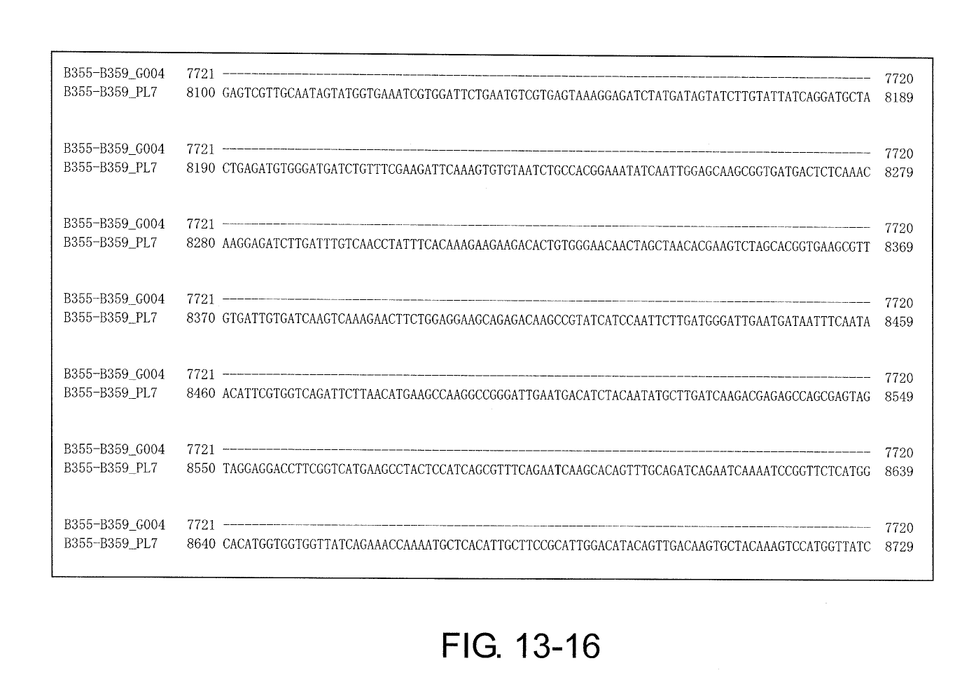

FIG. 13-17 is a continuation of FIG. 13-16.

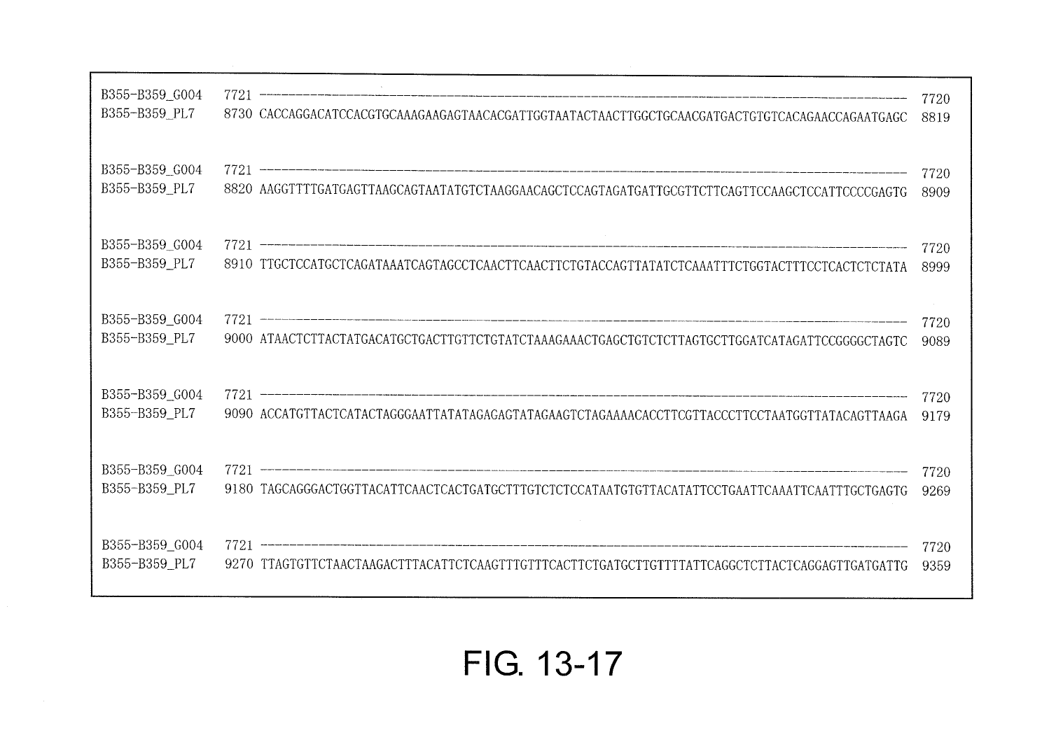

FIG. 13-18 is a continuation of FIG. 13-17.

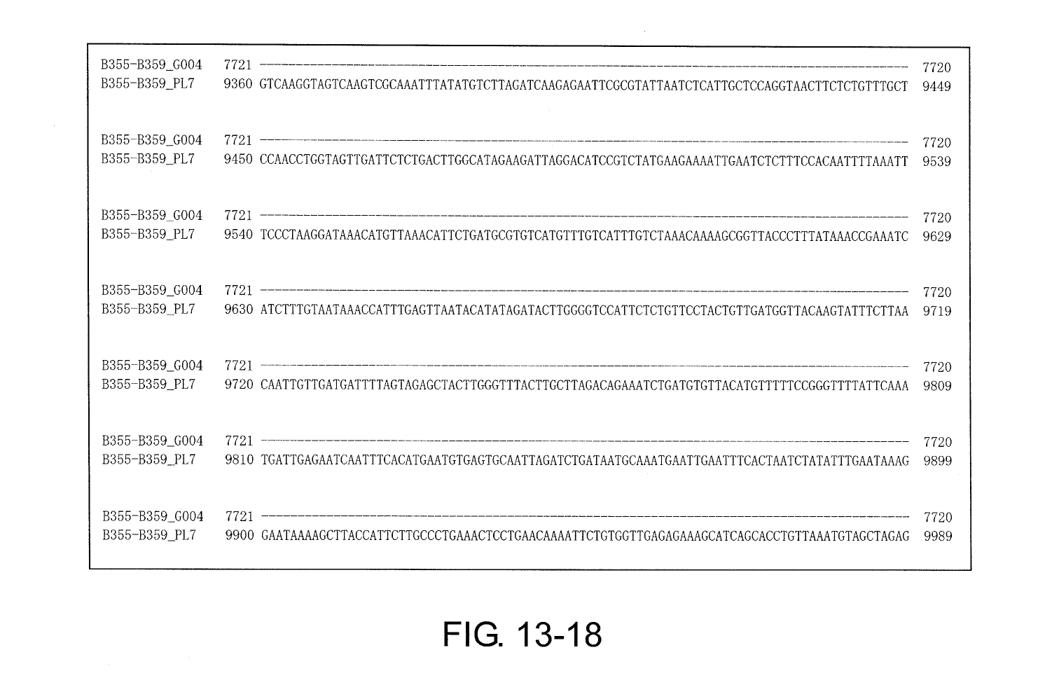

FIG. 13-19 is a continuation of FIG. 13-18.

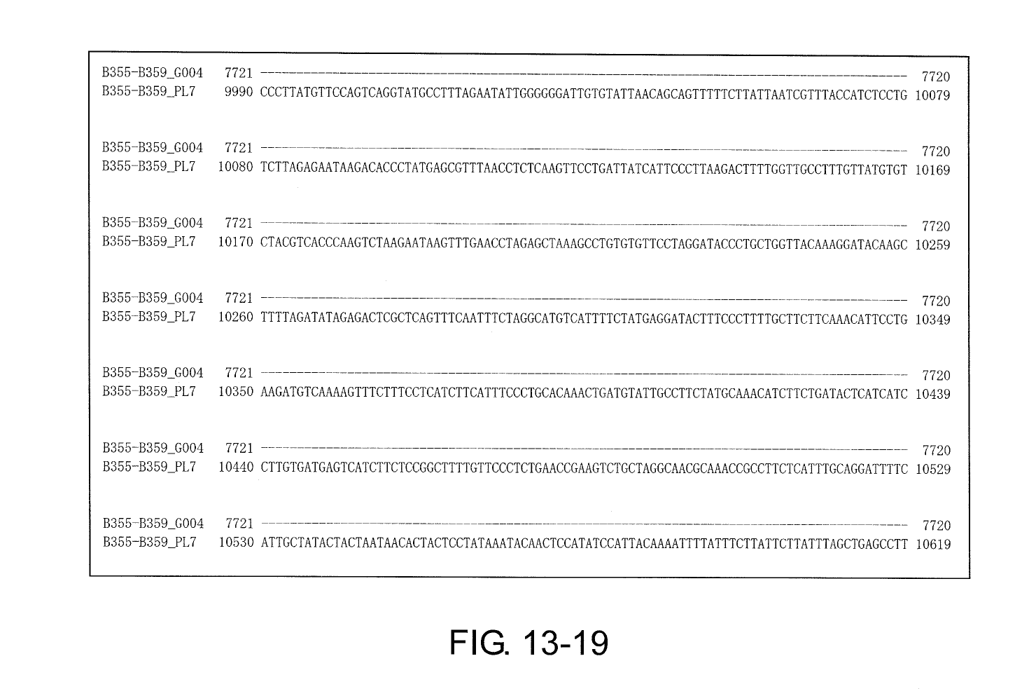

FIG. 13-20 is a continuation of FIG. 13-19.

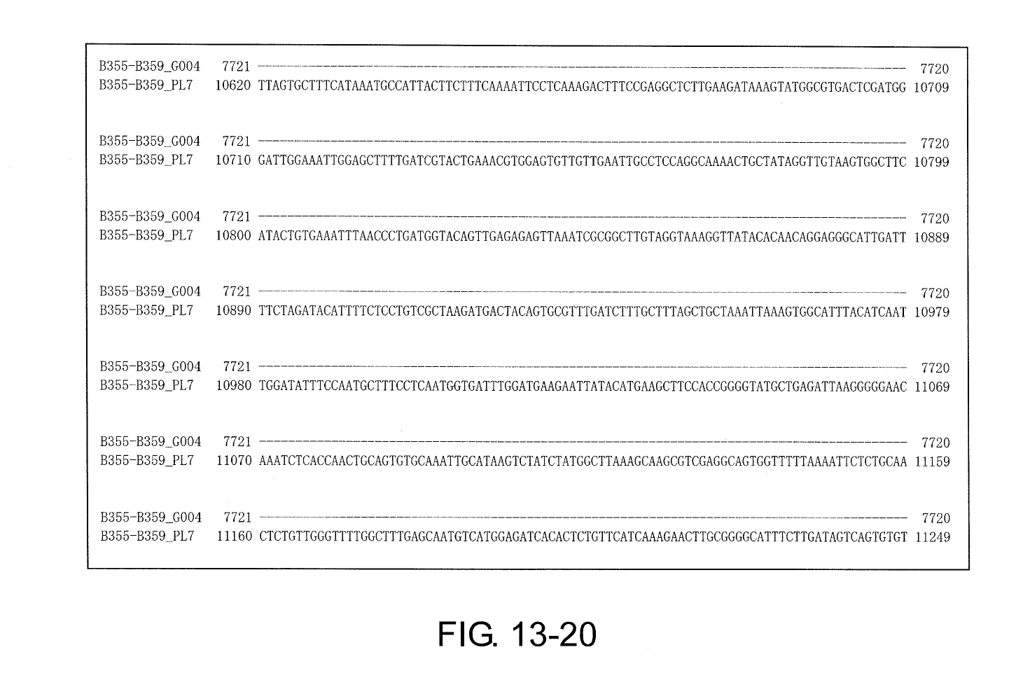

FIG. 13-21 is a continuation of FIG. 13-20.

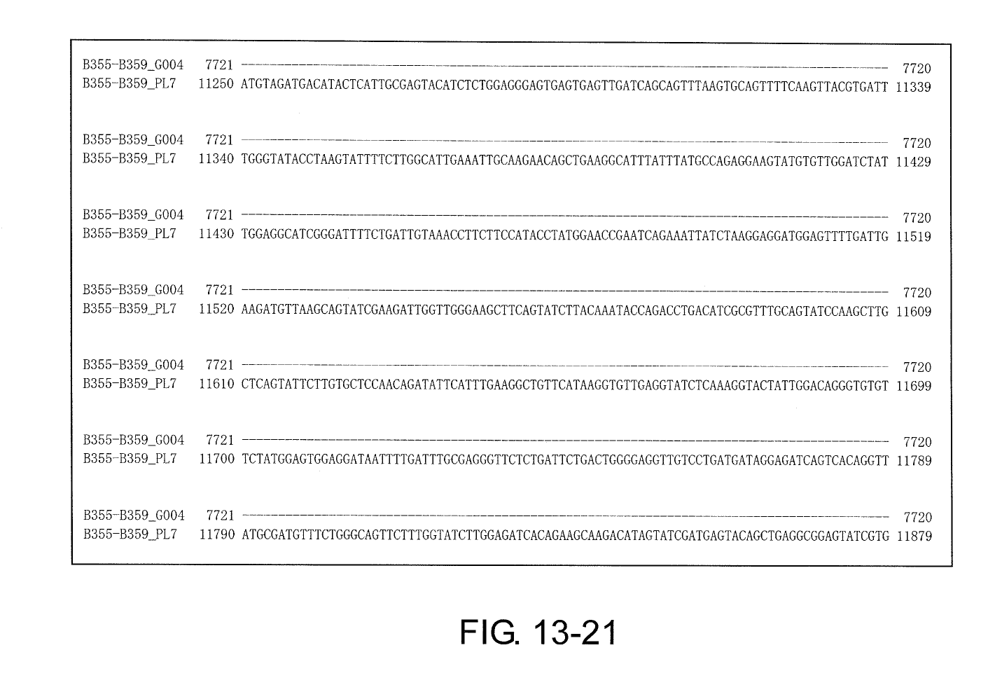

FIG. 13-22 is a continuation of FIG. 13-21.

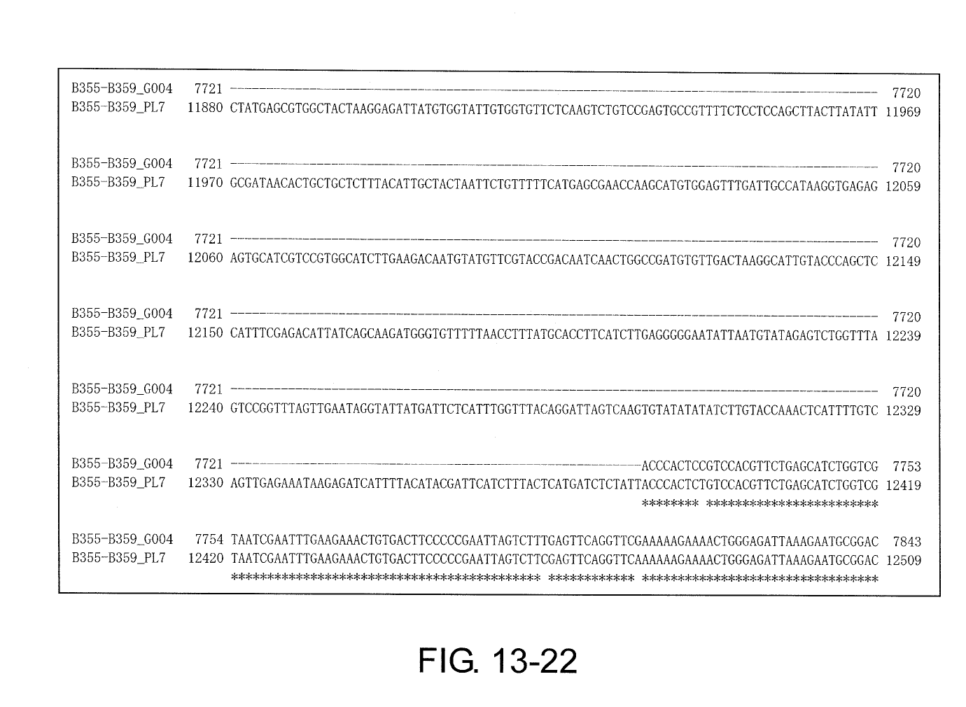

FIG. 13-23 is a continuation of FIG. 13-22.

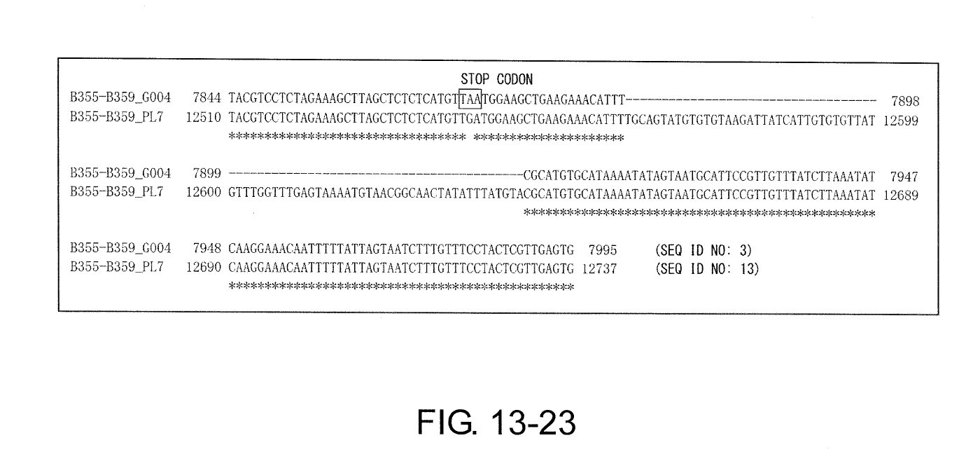

### MODE FOR CARRYING OUT THE INVENTION

The present inventors isolated genes conferring clubroot fungus resistance. A preferred embodiment of the above-mentioned genes of the present invention includes, for example, the Crr1 gene of Chinese cabbage.

In the present invention, “having clubroot fungus resistance” not only means that the subject plants have resistance to clubroot fungus, but also refers to conferring to the subject plants resistance against clubroot fungus.

The genomic sequence of the Crr1 gene identified by the present invention is shown in SEQ ID NO: 3, the cDNA nucleotide sequence is shown in SEQ ID NO: 1, and the amino acid sequence encoded by the nucleotide sequence is shown in SEQ ID NO: 2.

Specifically, the present invention provides a polynucleotide having clubroot fungus resistance, which is described in any one of (a) to (d) below:

(a) a polynucleotide encoding a protein comprising the amino acid sequence of SEQ ID NO: 2;

(b) a polynucleotide comprising the coding region of the nucleotide sequence of SEQ ID NO: 1;

(c) a polynucleotide encoding a protein comprising an amino acid sequence with one or more amino acid substitutions, deletions, additions, and/or insertions in the amino acid sequence of SEQ ID NO: 2; and

(d) a polynucleotide that hybridizes under stringent conditions to a complementary strand of the nucleotide sequence of SEQ ID NO: 1 or SEQ ID NO: 3.

In the present invention, a polynucleotide of any one of (a) to (d) mentioned above may be described as a “polynucleotide of the present invention”.

Furthermore, the term “polynucleotide” used in the present invention refers to a ribonucleotide or a deoxynucleotide, and indicates a polymer comprising multiple bases or base pairs. Single-stranded and double-stranded DNAs are included in polynucleotides. Polynucleotides include those that are not modified as well as those that are modified from the naturally-occurring state. Examples of modified bases include tritylated bases and unconventional bases such as inosine.

The term “nucleic acid” in the present invention means RNA or DNA. Furthermore, chemosynthetic nucleic acid analogs such as the so-called PNAs (peptide nucleic acids) are also included in the nucleic acid of the present invention. PNA is a molecule in which the pentose-phosphate backbone which is the basic backbone structure of a nucleic acid is substituted with a polyamide backbone composed of glycine units, and has a three-dimensional structure very similar to that of nucleic acids.

Furthermore, polynucleotides having clubroot fungus resistance of the present invention are not necessarily limited to polynucleotides comprising a nucleotide sequence specifically described in the Sequence Listing, or polynucleotides encoding a protein comprising an amino acid sequence specifically described in the Sequence Listing.

Proteins other than those described above are included in the proteins of the present invention, for example, when they are highly homologous (ordinarily, 70% or higher, preferably 80% or higher, more preferably 90% or higher, most preferably 95%, 96%, 97%, 98%, 99%, or higher homology) to the sequences described in the Sequence Listing, and maintain the functions (for example, clubroot fungus resistance) possessed by the proteins of the present invention.

Polynucleotides of the present invention include, for example, endogenous polynucleotides (homologs, etc.) in other organisms (plants), which correspond to a polynucleotide comprising the nucleotide sequence of SEQ ID NO: 1.

Furthermore, endogenous polynucleotides in other organisms, which correspond to a polynucleotide comprising the nucleotide sequence of SEQ ID NO: 1, are generally highly homologous to the polynucleotide of SEQ ID NO: 1. Highly homologous means a homology of 50% or higher, preferably 70% or higher, more preferably 80% or higher, and even more preferably 90% or higher (for example, 95% or higher, or even 96%, 97%, 98%, or 99% or higher). Such homology can be determined using the mBLAST algorithm (Altschul et al. (1990) Proc. Natl. Acad. Sci. USA 87: 2264-8; Karlin and Altschul (1993) Proc. Natl. Acad. Sci. USA 90: 5873-7).

Furthermore, when the polynucleotides are isolated from a living body, they are thought to hybridize under stringent conditions with the polynucleotide of SEQ ID NO: 1. Here, “stringent conditions” include, for example, conditions of “2×SSC, 0.1% SDS, 50° C.”, “2×SSC, 0.1% SDS, 42° C.”, and “1×SSC, 0.1% SDS, 37° C.”, and more stringent conditions include conditions of “2×SSC, 0.1% SDS, 65° C.”, “0.5×SSC, 0.1% SDS, 42° C.”, and “0.2×SSC, 0.1% SDS, 65° C.”. Based on the nucleotide sequence of SEQ ID NO: 1, those skilled in the art can appropriately obtain endogenous polynucleotides in other organisms that are equivalent to the polynucleotide of SEQ ID NO: 1.

The present invention also includes proteins encoded by the polynucleotides of the present invention. In the present invention, proteins encoded by the polynucleotides of the present invention may be described as “proteins of the present invention”.

The term “proteins” used in the present invention means polymers formed from multiple amino acids. Therefore, proteins of the present invention also include the so-called “polypeptides” and “oligopeptides”. Proteins of the present invention include those that are not modified as well as those that are modified from the naturally-occurring state. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, γ-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Proteins of the present invention can be produced by general chemical synthesis methods according to their amino acid sequences, and such methods include peptide synthesis methods by common liquid-phase methods and solid-phase methods. More specifically, such peptide synthesis methods may include the stepwise elongation method, in which each amino acid is successively synthesized one by one based on the amino acid sequence information to lengthen the chain, and the fragment condensation method, in which fragments containing several amino acids are synthesized in advance, and then each of these fragments are subjected to coupling reactions. Either method may be used for the synthesis of the proteins of the present invention.

Condensation methods used in such peptide synthesis methods can be carried out according to various types of methods, and examples include the azide method, mixed acid anhydride method, DCC method, active ester method, oxidation-reduction method, diphenylphosphoryl azide (DPPA) method, and Woodward method.

For solvents to be used in these various methods, generally used solvents can be suitably used. Such examples include dimethylformamide (DMF), dimethylsulfoxide (DMSO), hexaphosphoroamide, dioxane, tetrahydrofuran (THF), ethyl acetate, and mixed solvents thereof. During the above-mentioned peptide synthesis reaction, the carboxyl groups of amino acids and peptides that are not involved in the reaction can generally be protected by esterification, for example, as lower alkyl esters such as methyl ester, ethyl ester, or tertiary butyl ester, or as benzyl ester, p-methoxybenzyl ester, orp-nitrobenzyl ester aralkyl ester. Furthermore, the hydroxyl group of an amino acid having a functional group on its side chain, for example, Tyr, may be protected by an acetyl group, a benzyl group, a benzyloxycarbonyl group, a tertiary butyl group, or such, but such protection is not necessarily essential. Furthermore, for example, Arg can have its guanidino group protected by a suitable protecting group such as a nitro group, tosyl group, 2-methoxybenzenesulfonyl group, mesitylene-2-sulfonyl group, benzyloxycarbonyl group, isobornyloxycarbonyl group, or adamantyloxycarbonyl group.

Proteins of the present invention include, for example, proteins that are functionally equivalent to the proteins of the present invention. Herein, “functionally equivalent” means that the protein of interest has a biological or biochemical function (activity) similar or equivalent to that of a protein of the present invention. Examples of such functions include clubroot fungus resistance or clubroot fungus resistance activity.

The most common method for evaluating whether or not a certain polynucleotide encodes a protein having clubroot fungus resistance is, for example, the method of cultivating a plant that has been introduced with the polynucleotide and evaluating the plant's level of resistance to clubroot fungus.

Examples of methods for preparing a protein functionally equivalent to a certain protein that are well known to those skilled in the art include methods for introducing mutations into the amino acid sequence of a protein. For example, those skilled in the art can prepare a protein functionally equivalent to the above-mentioned proteins by introducing appropriate mutations into the amino acid sequence of SEQ ID NO: 2 using site-directed mutagenesis or such (Hashimoto-Gotoh, T. et al. (1995) Gene 152, 271-275; Zoller, M J, and Smith, M. (1983) Methods Enzymol. 100, 468-500; Kramer, W. et al. (1984) Nucleic Acids Res. 12, 9441-9456; Kramer W, and Fritz H J (1987) Methods. Enzymol. 154, 350-367; Kunkel, T A (1985) Proc. Natl. Acad. Sci. USA. 82, 488-492 and Kunkel (1988) Methods Enzymol. 85, 2763-2766). Amino acid mutations in a protein may also occur naturally. Regardless of whether they are artificial or naturally-occurring, proteins functionally equivalent to the above-mentioned proteins, which comprise an amino acid sequence in which one or more amino acid sequences are mutated in the amino acid sequence of SEQ ID NO: 2, are included in the proteins of the present invention.

The above-mentioned proteins are not limited as long as they maintain the functions possessed by the proteins of the present invention, and include, for example, a protein comprising an amino acid sequence with one or more amino acid additions, deletions, substitutions, or insertions in the amino acid sequence of SEQ ID NO: 2. The number of modified amino acids is not particularly limited as long as the modified proteins have the aforementioned functions, but are ordinarily 50 amino acids or less, preferably 30 amino acids or less, and more preferably ten amino acids or less (for example, five amino acids or less, or three amino acids or less). Alternatively, in the entire amino acid sequence, for example, modifications of 20% or less, or more specifically 10% or less (for example, 10, 9, 8, 7, 6, 5, 4, 3, 2, or 1% or less) of the amino acid residues are acceptable. That is, proteins containing amino acid sequences that have homology of preferably 80% or more, or more preferably 90% or more (for example, 90, 91, 92, 93, 94, 95, 96, 97, 98, or 99% or more) to the amino acid sequence of SEQ ID NO: 2 are also included in the proteins of the present invention.

Generally, to maintain protein function, amino acids to be substituted are preferably amino acids that have properties similar to those of the amino acids before substitution. Such amino acid residue substitutions are called conservative substitutions. For example, since Ala, Val, Leu, Ile, Pro, Met, Phe, and Trp are all classified as non-polar amino acids, their properties are similar to each other. Uncharged amino acids include Gly, Ser, Thr, Cys, Tyr, Asn, and Gln. Furthermore, acidic amino acids include Asp and Glu. Furthermore, basic amino acids include Lys, Arg, and His. Amino acid substitutions within each of these groups are acceptable.

The amino acid residues to be mutated are desirably mutated to other amino acids in which the properties of the amino acid side chain are conserved. Examples of amino acid side chain properties are: hydrophobic amino acids (A, I, L, M, F, P, W, Y, and V), hydrophilic amino acids (R, D, N, C, E, Q, G, H, K, S, and T), and amino acids having the following side chains: aliphatic side chains (G, A, V, L, I, and P); hydroxyl-containing side chains (S, T, and Y); sulfur-containing side chains (C and M); carboxylic acid- and amide-containing side chains (D, N, E, and Q); basic side chains (R, K, and H); and aromatic ring-containing side chains (H, F, Y, and W) (all amino acids are represented by one-letter codes in parentheses).

A protein having a modified amino acid sequence, in which one or more amino acid residues are deleted, added, and/or substituted with other amino acids in a certain amino acid sequence, is known to be able to retain its biological function (activity) (Mark, D. F. et al., Proc. Natl. Acad. Sci. USA (1984) 81, 5662-5666; Zoller, M. J. & Smith, M. Nucleic Acids Research (1982) 10, 6487-6500; Wang, A. et al., Science 224, 1431-1433; and Dalbadie-McFarland, G. et al., Proc. Natl. Acad. Sci. USA (1982) 79, 6409-6413).

When a specific amino acid sequence (for example SEQ ID NO: 2) is disclosed, those skilled in the art can appropriately select a protein of the present invention by appropriately producing a protein comprising the amino acid-modified sequence based on this amino acid sequence, and evaluating whether or not the protein has the desired function.

Proteins in which several amino acid residues have been added to an amino acid sequence of a protein of the present invention include fusion proteins containing these proteins. Fusion proteins are proteins in which such a protein is fused to another protein. A fusion protein can be prepared by a method that ligates a polynucleotide (for example, SEQ ID NO: 1) encoding a protein of the present invention (for example, SEQ ID NO: 2) to a polynucleotide encoding another protein such that their frames are in line, inserts this into an expression vector, and expresses it in a host; and techniques known to those skilled in the art can be used. The other peptide or polypeptide to be fused with a protein of the present invention is not particularly limited.

Examples of other proteins to be fused to the proteins of the present invention include, GST (glutathione-S-transferase), immunoglobulin constant region, β-galactosidase, MBP (maltose-binding protein), and such. Commercially available polynucleotides encoding these proteins can be fused with polynucleotides encoding the proteins of the present invention. A fusion protein can be prepared by expressing the fusion polynucleotide prepared in this way.

Other methods that are well known to those skilled in the art for preparing proteins that are functionally equivalent to a certain protein include, for example, a method that uses hybridization techniques (Sambrook, J. et al., Molecular Cloning 2nd ed., 9.47-9.58, Cold Spring Harbor Lab. press, 1989). More specifically, based on the polynucleotide (the nucleotide sequence of SEQ ID NO: 1) encoding a protein of the present invention or a portion thereof, those skilled in the art can generally isolate polynucleotides highly homologous thereto from polynucleotide samples derived from organisms of the same or different species, and then from these polynucleotides, isolate proteins functionally equivalent to the proteins of the present invention.

The present invention includes proteins that are encoded by a polynucleotide which hybridizes with the polynucleotide encoding a protein of the present invention, and which are functionally equivalent to a protein of the present invention.

Those skilled in the art can appropriately select hybridization conditions for isolating polynucleotides encoding proteins that are functionally equivalent to proteins of the present invention. The hybridization conditions are, for example, low-stringency conditions. “Low-stringency conditions” refers to washing after hybridization under conditions such as 42° C., 0.1×SSC, 0.1% SDS, or preferably 50° C., 0.1×SSC, 0.1% SDS. More preferable hybridization conditions include high-stringency conditions. High-stringency conditions are, for example, conditions of 65° C., 5×SSC, and 0.1% SDS. Under these conditions, increasing the temperature is expected to result in efficient yield of DNAs having higher homology. However, multiple factors such as temperature and salt concentration are considered to be factors affecting the hybridization stringency, and those skilled in the art can achieve similar stringencies by appropriately selecting these factors.

In place of hybridization, a gene amplification technique (PCR) may be used to isolate a polynucleotide fragment highly homologous to a polynucleotide encoding a protein of the present invention by designing primers based on a portion of the polynucleotide (for example, SEQ ID NO: 1) encoding a protein of the present invention (Current protocols in Molecular Biology edit. Ausubel et al., (1987) Publish. John Wiley & Sons Section 6.1-6.4); and based on this polynucleotide, a protein functionally equivalent to a protein of the present invention can be obtained.

The proteins of the present invention may be in the form of a “mature” protein, or may be a part of a larger protein such as a fusion protein. The proteins of the present invention may contain leader sequences, pro-sequences, sequences which are useful in purification, such as multiple histidine residues, or additional sequences for securing stability during recombinant production.

Proteins functionally equivalent to proteins of the present invention, which are encoded by polynucleotides isolated by the above-mentioned hybridization techniques or gene amplification techniques, generally have high amino acid sequence homology with proteins of the present invention (for example, SEQ ID NO: 2). Proteins that are functionally equivalent to the proteins of the present invention, and have high amino acid sequence homology with these proteins are also included in the proteins of the present invention. High homology usually refers to an identity at the amino acid level of at least 50% or higher, preferably 75% or higher, more preferably 85% or higher, and even more preferably 95% or higher (for example, 96% or higher, 97% or higher, 98% or higher, or 99% or higher). Protein homology can be determined by following the algorithm described in the literature (Wilbur, W. J. and Lipman, D. J. Proc. Natl. Acad. Sci. USA (1983) 80, 726-730).

The amino acid sequence identity can be determined, for example, by the BLAST algorithm of Karlin and Altschul (Proc. Natl. Acad. Sci. USA 87, :2264-2268, 1990; Proc. Natl. Acad. Sci. USA, 90: 5873-5877, 1993). A program called BLASTX has been developed based on this algorithm (Altschul et al., J. Mol. Biol. 215: 403-410, 1990). When amino acid sequences are analyzed by BLASTX, parameters are set, for example, at score=50 and wordlength=3. When using the BLAST and Gapped BLAST programs, the default parameters of each program are used. Specific procedures for these analytical methods are known (http://www.ncbi.nlm.nih.gov).

The proteins of the present invention can be prepared as a recombinant protein or as a naturally-occurring protein by methods known to those skilled in the art. Recombinant proteins can be prepared, for example, by incorporating a polynucleotide encoding a protein of the present invention (for example, the nucleotide sequence of SEQ ID NO: 1) into a suitable expression vector, harvesting transformants obtained by introducing this vector into suitable host cells, and obtaining their extracts and appropriately purifying them according to common methods generally used in the field of peptide chemistry such as ion exchange resin, partition chromatography, gel chromatography, affinity chromatography, high performance liquid chromatography (HPLC), and countercurrent distribution method.

When proteins of the present invention are expressed as fusion proteins with a glutathione S-transferase protein, or as recombinant proteins with multiple additions of histidines in host cells (for example, a plant cell or a microbial cell), the expressed recombinant proteins can be purified using a glutathione column or a nickel column. After the fused protein is purified, regions other than the protein of interest in the fused protein can be removed, as necessary, by cleavage with thrombin, factor Xa, or such.

Naturally derived proteins can be isolated by methods well known to those skilled in the art, for example, by purifying extracts of tissues or cells expressing the proteins of the present invention by subjecting them to an affinity column to which antibodies having affinity to the proteins of the present invention are bound. The antibodies that are used may be polyclonal antibodies or monoclonal antibodies.

Proteins of the present invention can be utilized, for example, in the production of antibodies that recognize the proteins of the present invention and such.

Polynucleotides of the present invention may be in any form as long as they can encode proteins of the present invention. That is, whether the polynucleotides are cDNAs synthesized from mRNAs, genomic DNAs, chemically synthesized DNAs, or such is not a concern. Furthermore, as long as a protein of the present invention is encoded, DNAs having an arbitrary nucleotide sequence based on genetic code degeneracy are also included.

Polynucleotides of the present invention can be prepared by methods known to those skilled in the art. For example, they can be prepared by producing a cDNA library from cells expressing the proteins of the present invention, then performing hybridization using a portion of the polynucleotides of the present invention (for example, the nucleotide sequence of SEQ ID NO: 1) as a probe. The cDNA library may be prepared, for example, by a method described in the literature (Sambrook, J. et al., Molecular Cloning, Cold Spring Harbor Laboratory Press (1989)), or a commercially available DNA library may be used. Alternatively, polynucleotides can be produced by preparing RNAs from cells expressing a protein of the present invention, synthesizing cDNAs using reverse transcriptase, and then synthesizing oligoDNAs based on the polynucleotides of the present invention (for example, the nucleotide sequence of SEQ ID NO: 1), and performing PCR reactions using them as primers to amplify the cDNAs encoding the proteins of the present invention.

By determining the nucleotide sequence of the obtained cDNA, the translation region encoded by the cDNA can be determined, and the amino acid sequence of the protein of the present invention can be obtained. Furthermore, the obtained cDNA can also be used as a probe for screening a genomic DNA library to isolate genomic DNAs.

Specifically, the following processes may be carried out. First, mRNAs are isolated from cells, tissues, or organs expressing a protein of the present invention. mRNAs are isolated using known methods, for example, by preparing total RNAs using guanidine ultracentrifugation methods (Chirgwin, J. M. et al., Biochemistry (1979) 18, 5294-5299), AGPC methods (Chomczynski, P. and Sacchi, N., Anal. Biochem. (1987) 162, 156-159), or such, and then purifying mRNAs from the total RNAs using an mRNA Purification Kit (Pharmacia) or such. The mRNAs can also be prepared directly by using the QuickPrep mRNA Purification Kit (Pharmacia).

cDNAs are synthesized from the obtained mRNAs using reverse transcriptase. cDNAs may be synthesized using the AMV Reverse Transcriptase First-strand cDNA Synthesis Kit (Seikagaku Corporation) and such. Alternatively, by using the primers and such described herein, cDNAs may be synthesized and amplified following the 5′-RACE method (Frohman, M. A. et al., Proc. Natl. Acad. Sci. USA (1988) 85, 8998-9002; Belyaysky, A. et al., Nucleic Acids Res. (1989) 17, 2919-2932) that uses the 5′-Ampli FINDER RACE Kit (manufactured by Clontech) and polymerase chain reaction (PCR). A polynucleotide fragment of interest is prepared from the obtained PCR products and linked to a vector. A recombinant vector is produced from this and introduced into E. coli and such, and colonies are selected to prepare a desired recombinant vector. The nucleotide sequence of the polynucleotide of interest can be confirmed through known methods such as the dideoxynucleotide chain termination method.

Furthermore, when producing polynucleotides of the present invention, the nucleotide sequences having higher expression efficiency can be designed by considering the codon usage frequency in the host used for expression (Grantham R. et al., Nucleic Acids Research (1981) 9, r43-74). Furthermore, polynucleotides of the present invention can be modified by commercially available kits or known methods. Examples of the modification include digestion with restriction enzymes, insertion of a synthetic oligonucleotide or a suitable DNA fragment, addition of a linker, and insertion of the initiation codon (ATG) and/or a stop codon (TAA, TGA, or TAG).

Furthermore, the present invention provides vectors into which polynucleotides of the present invention have been inserted.

In addition to the above-mentioned vectors used for recombinant protein production, vectors of the present invention include vectors for expressing polynucleotides of the present invention in plant cells for the production of transformed plants. Examples of preferred embodiments of the present invention include vectors in which a polynucleotide of the present invention is operably linked downstream of a promoter region that enables expression in plant cells. For example, they can contain a promoter sequence that enables transcription in plant cells and a terminator sequence containing a polyadenylation site necessary for stabilization of transcription products. A vector used for transformation of a plant cell is not particularly limited as long as it allows expression of an inserted gene in the cell. For example, a vector having a promoter for constitutively expressing a gene in plant cells, and a vector having a promoter that is inducibly activated by an external stimulus may be used.

A promoter for constitutively expressing a protein of the present invention may be, for example, the ubiquitin promoter from lettuce, the 35S promoter from cauliflower mosaic virus, the actin promoter from rice, or the ubiquitin promoter from maize.

Promoters for inducible expression include, for example, promoters known to be expressed by exogenous factors including bacterial or viral infection or invasion, low temperature, elevated temperature, dryness, UV light radiation, and application of specific compounds. Examples of such promoters include the rice chitinase gene promoter and tobacco PR protein gene promoter which are expressed by bacterial or viral infection or invasion; the rice “lip19” gene promoter induced by low temperature; the rice “hsp80” gene and “hsp72” gene promoter induced by high temperature; the Arabidopsis thaliana “rab16” gene promoter induced by dryness; the parsley chalcone synthase gene promoter induced by UV light radiation; the maize alcohol dehydrogenase gene promoter induced by anaerobic conditions; and such. Also, the rice chitinase gene promoter and tobacco PR protein gene promoter are induced by specific compounds such as salicylic acid, and “rab16” is also induced by application of the phytohormone abscisic acid.

Those skilled in the art can appropriately produce vectors carrying desired polynucleotides using general genetic engineering techniques. Usually, various commercially available vectors can be used.

Vectors of the present invention are also useful for retaining polynucleotides of the present invention in host cells, and expressing proteins of the present invention.

Polynucleotides of the present invention are generally carried by (inserted into) suitable vectors and then introduced into host cells. The vectors are not particularly limited as long as the inserted polynucleotide is stably maintained. For example, when using E. coli as a host, pBluescript vector (manufactured by Stratagene) and such are preferable as cloning vector, but various commercially available or known vectors can be used. Expression vectors are particularly useful when using vectors for the purpose of producing proteins of the present invention. Expression vectors are not particularly limited as long as they can express proteins in test tubes, E. coli, cultured cells, or individual plants. For example, such vectors are pBEST vector (manufactured by Promega) for expression in test tubes, pET vector (manufactured by Invitrogen) for E. coli, pME18S-FL3 vector (GenBank Accession No. AB009864) for cultured cells, and pME18S vector (Mol. Cell Biol. 8, 466-472 (1988)) for individual organisms. Insertion of a polynucleotide of the present invention into vectors can be performed by standard methods such as ligase reactions using restriction enzyme sites.

The above-mentioned host cells are not particularly limited, and various host cells can be used depending on the purpose. Cells used for expressing the proteins of the present invention include bacterial cells (for example, Streptococcus, Staphylococcus, E. coli, Streptomyces, and Bacillus subtilis), insect cells (for example, Drosophila S2 and Spodoptera SF9), animal cells (for example, CHO, COS, HeLa, C127, 3T3, BHK, HEK293, Bowes melanoma cell), and plant cells. Vectors can be introduced into host cells using known methods such as the calcium phosphate precipitation method, electroporation method (Current protocols in Molecular Biology edit. Ausubel et al. (1987) Publish. John Wiley & Sons Section 9.1-9.9), lipofection method (manufactured by GIBCO-BRL), and microinjection method.

To secrete host cell-expressed proteins into the lumen of endoplasmic reticulum, periplasmic space, or extracellular environment, suitable secretion signals can be incorporated into the proteins of interest. These signals may be endogenous or heterogenous to the proteins of interest.

When the proteins of the present invention are secreted into culture media, the media are collected. When the proteins of the present invention are produced inside cells, the cells are first lysed, and then the proteins are collected.

The proteins of the present invention can be collected and purified from recombinant cell cultures using known methods, including ammonium sulfate or ethanol precipitation, acidic extraction, anion or cation exchange chromatography, phosphocellulose chromatography, hydrophobic interaction chromatography, affinity chromatography, hydroxyapatite chromatography, and lectin chromatography.

Methods for expressing a polynucleotide of the present invention in a plant include the method of incorporating a polynucleotide of the present invention into a suitable vector and then introducing this into a living body by methods such as the electroporation method, agrobacterium method, liposome method, cationic liposome method, and such.

General genetic engineering procedures such as insertion of a polynucleotide of the present invention into a vector can be carried out according to conventional procedures (Molecular Cloning, 5.61-5.63). Administration into a plant may be performed by an ex vivo method or an in vivo method. A method for introducing a polynucleotide of the present invention into a plant is, for example, an Agrobacterium-mediated method for introducing a gene.

Furthermore, using a technique described later, one can produce a transformed plant into which a polynucleotide of the present invention has been introduced, and a protein of the present invention can be prepared from this plant.

Furthermore, by using recombinant proteins obtained as described above, one can prepare antibodies that bind to them. For example, one can prepare polyclonal antibodies by immunizing animals such as rabbits with purified proteins of the present invention or a partial peptide thereof, collecting blood after a certain period of time, and removing blood clots.

Further, one can prepare monoclonal antibodies by fusing antibody-producing cells of animals immunized with the above-mentioned proteins or peptides with bone tumor cells, isolating single-clone cells (hybridoma) producing the antibodies of interest, and obtaining antibodies from the cells. The antibodies thus obtained can be used for purification and detection of the proteins of the present invention. The present invention includes antibodies that bind to the proteins of the present invention. By using these antibodies, it is possible to determine the location where the proteins of the present invention are expressed in plants or determine whether or not a plant species expresses a protein of the present invention.

The present invention relates to transformed plant cells into which a vector of the present invention described above has been introduced. When transformed plants having clubroot resistance activity are produced using the polynucleotides of the present invention, a polynucleotide encoding a protein of the present invention is inserted into a suitable vector, the vector is introduced into a plant cell, and an obtained transformed plant cell is regenerated.

By introducing a protein of the present invention into an arbitrary plant species and expressing it, it is possible to confer clubroot resistance activity to those plants or their seeds. The time required for this transformation is very short as compared to gene transfer by conventional crossing. It is also advantageous since it does not involve other phenotypic changes.

Cells into which a vector of the present invention is introduced include, in addition to the above-mentioned cells used for producing recombinant proteins, plant cells used for generating transformed plants.

In the present invention, the “plants” are not particularly limited, but are preferably plants which may become infected with clubroot fungus, for example, cruciferous plants. Specific examples include Chinese cabbage (Hakusai, Brassica rapa L. Pekinensis group), turnip, Bok choy, Nabana (Brassica napus L., Brassica rapa L. Oleifera Group), Nozawa-na (Brassica rapa L. Hakabura Group), Tsukena (Brassica rapa L. Perviridis Group), cabbage, broccoli, cauliflower, rapeseed, daikon radish (Raphanus sativus L. Daikon Group), and such. The “plant cells” include, in addition to cells within plants, plant cells in various forms including cultured cells (for example, suspension culture cells), protoplasts, shoot primordia, multiple shoots, hairy roots, sections of leaves, calli, and such.

Vectors can be introduced into plant cells by using various methods known to those skilled in the art, such as polyethylene glycol methods, electroporation, Agrobacterium-mediated methods, and particle gun methods. Plants can be regenerated from transformed plant cells using methods known to those skilled in the art according to the type of plant cell. Several techniques have already been established, such as the method of introducing genes into protoplasts using polyethylene glycol and regenerating the plant, the method of introducing genes into protoplasts using electric pulse and regenerating the plant, the method of introducing genes directly into cells by the particle gun method and regenerating the plant, and the method of introducing genes via Agrobacterium and regenerating the plant; and they are widely used in the technical field of the invention of this application. These methods can be suitably used in the present invention.

For efficient selection of plant cells transformed by introduction of a vector of the present invention, preferably the above-mentioned vector of the present invention contains a suitable selection marker gene or is introduced into the plant cell together with a plasmid vector containing a selection marker gene. Examples of selection marker genes used for this purpose include the hygromycin phosphotransferase gene for resistance to the antibiotic hygromycin, neomycin phosphotransferase gene for resistance to kanamycin or gentamycin, and acetyl transferase gene for resistance to the herbicide phosphinothricin.

Plant cells introduced with a recombinant vector are placed in a known selection medium containing a suitable agent for selection according to the type of the introduced selection marker gene, and the cells are cultured. This way, transformed cultured plant cells can be obtained.

Plants can be regenerated by redifferentiating transformed plant cells. The method of redifferentiation differs depending on the type of plant cells, and examples include the method of Takasaki et al. (Breeding Sci. 47: 127-134 (1997)) for Japanese mustard spinach (Brassica rapa Perviridis Group) which can be crossed with Chinese cabbage, the method of Fujimura et al. (Plant Tissue Culture Lett. 2: 74 (1995)) for rice, and the methods of Shillito et al. (Bio/Technology 7: 581 (1989)) and Gorden-Kamm et al. (Plant Cell 2: 603 (1990)) for maize.

Once a transformed plant that has a polynucleotide of the present invention integrated into the genome is obtained, it is possible to obtain a progeny from the plant by sexual or asexual reproduction. It is also possible to obtain propagation materials (breeding materials such as seeds, fruits, panicles, tubers, root tubers, stubs, calluses, and protoplasts) from the plant or a progeny or clone thereof, and mass-produce the plant based on such material. Thus, the present invention includes plant cells into which a polynucleotide of the present invention has been introduced, plants containing these cells, progenies and clones of these plants, as well as propagation materials and breeding materials of the plants, their progenies and clones. Plants produced in this manner or their seeds are expected to have clubroot resistance activity.

The present invention provides methods for assessing clubroot fungus resistance in a test plant or a test propagation medium. Specifically, the present invention provides a method for assessing clubroot fungus resistance in a test plant or a test propagation medium, which contains the step of detecting a DNA region containing the nucleotide sequence of SEQ ID NO: 1 or SEQ ID NO:3 or a partial sequence or surrounding sequence thereof. For example, a method including steps of (i) to (iii) below may be carried out:

(i) preparing a DNA sample from a test plant or a test propagation medium;

(ii) amplifying a DNA region comprising the nucleotide sequence of SEQ ID NO: 1 or SEQ ID NO: 3, or a partial sequence or surrounding sequence thereof, from the DNA sample; and

(iii) comparing the molecular weight or the nucleotide sequence of a DNA fragment produced by amplifying the DNA region comprising the nucleotide sequence of SEQ ID NO: 1 or 3, or a partial sequence or surrounding sequence thereof, from the clubroot fungus-resistant plant or propagation medium with that of the DNA fragment amplified in step (ii).

In the present invention, “assessing clubroot resistance” includes not only assessment of clubroot resistance in cultivars cultivated so far, but also assessment of clubroot resistance in novel cultivars produced by crossing or genetic recombination techniques.

Methods for assessing clubroot resistance of plants or propagation media of the present invention comprise detecting whether or not a plant or a propagation medium retains a functional clubroot resistance gene. Whether or not a plant or a propagation medium retains a functional clubroot resistance gene can be evaluated by detecting differences in the molecular weight or nucleotide sequence of regions in the cDNA or genomic DNA region that correspond to the clubroot resistance gene.

An embodiment is a method that compares the molecular weight of a DNA region that corresponds to the clubroot resistance gene in test plants and test propagation media (for example, a DNA region comprising the nucleotide sequence of SEQ ID NO: 1 or SEQ ID NO: 3, or a partial sequence or surrounding sequence of the nucleotide sequence), with that of the same DNA region in a plant or a propagation medium having clubroot resistance.

First, a DNA sample is prepared from a test plant or a propagation medium. Next, a DNA region that corresponds to the clubroot resistance gene (for example, a DNA region comprising the nucleotide sequence of SEQ ID NO: 1 or 3, or a partial sequence or surrounding sequence of this nucleotide sequence) is amplified from the DNA sample. Furthermore, the molecular weight of the DNA fragment produced by amplifying the DNA region of the clubroot resistance gene in a clubroot-resistant cultivar is compared with that of the DNA fragment amplified from the DNA sample; and when the molecular weight is significantly lower than that of the clubroot-resistant cultivar, the clubroot resistance of the test plant or propagation medium is determined to be decreased.

Specifically, first, the DNA region of a clubroot resistance gene of the present invention is amplified using methods such as the PCR method. The term “clubroot resistance gene” in the present invention refers to a part corresponding to the cDNA region (DNA region of SEQ ID NO: 1) or genomic DNA region (the DNA region of SEQ ID NO: 3) of a clubroot resistance gene, and the amplified range may be the cDNA region or full-length genomic DNA, or a part of the genomic DNA. The above-mentioned assessment method comprises using the nucleotide sequence of SEQ ID NO: 1 or SEQ ID NO: 3, or a partial sequence or surrounding sequence of the nucleotide sequence as DNA marker.

PCR can be performed by those skilled in the art by appropriately selecting the reaction conditions and such. The amplified DNA products can be labeled using primers labeled with an isotope such as ³²P, a fluorescent dye, biotin, or such during PCR. Alternatively, amplified DNA products can be labeled by adding substrate bases labeled with an isotope such as ³²P, fluorescent dye, or biotin to PCR reaction solutions, and performing PCR. Furthermore, labeling can also be carried out by adding to the amplified DNA fragments, substrate bases labeled with an isotope such as ³²P, a fluorescent dye, or biotin using Klenow enzyme or such after PCR reaction.

The labeled DNA fragment obtained this way is denatured by heating or such, and electrophoresed in polyacrylamide gels containing a denaturant such as urea or SDS. In the present invention, SDS-PAGE which uses SDS as denaturant is an advantageous separation technique, and SDS-PAGE can be performed according to Laemmli's method (Laemmli (1970) Nature 227, 680-685). After electrophoresis, mobility of the DNA fragment is analyzed and detected by autoradiography using X-ray films, fluorescence detection scanner, and the like. Even when labeled DNAs are not used, bands can be detected by staining the electrophoresed gel with ethidium bromide or by using the silver staining method. For example, clubroot resistance can be determined by amplifying DNA fragments from the clubroot-resistant cultivars and test plants using the polynucleotides of SEQ ID NOs: 9 to 12 as primers, and comparing their molecular weights.

Moreover, the clubroot resistance in a plant or propagation medium can be assessed by directly determining the nucleotide sequence of a test-plant DNA region that corresponds to a DNA of the present invention, and comparing it to the nucleotide sequences of clubroot-resistant cultivars. Clubroot resistance can be assessed in the following way. For example, in the range corresponding to the genomic region of SEQ ID NO: 3 in a test plant or propagation medium, when the 10 bp of positions 268 to 277 are deleted, 314 bp are inserted at position 395, the 5 bp of positions 740 to 744 are deleted, 3 bp are inserted at position 1286, 21 bp are inserted at position 1552, the 241 bp of positions 1784 to 2024 are deleted, 357 bp are inserted at position 2561, the 1061 bp of positions 3370 to 4430 are deleted, 326 bp are inserted at position 6803, 4981 bp are inserted at position 7721, or 76 bp are inserted at position 7898, the test plant or propagation medium has a clubroot-susceptible phenotype (decreased clubroot resistance). Alternatively, clubroot resistance can be assessed in the following way. When the genomic region of SEQ ID NO: 3 is amplified in a test plant or propagation medium, if the nucleotide sequence of SEQ ID NO: 13 is detected, the test plant or propagation medium has a clubroot-susceptible phenotype (decreased clubroot resistance).

The above-mentioned assessment step is not limited to assessment using gels such as agarose or polyacrylamide, and assessment methods that may be used by those skilled in the art such as assessment by capillary-type electrophoresis apparatus, or assessment using single nucleotide polymorphisms (SNPs) may be used.

Furthermore, the present invention also provides methods for selecting plants or seeds thereof having the clubroot resistance gene by an above-mentioned assessment method of the present invention.

As described above, a method for producing transformed plants, which comprises the step of introducing polynucleotides or vectors of the present invention to plant cells, and regenerating a plant from the plant cells is also included in the present invention. Specifically, the present invention relates to methods for producing plants or seeds thereof having clubroot resistance activity, which comprise the following steps of:

(i) introducing a vector of the present invention into plant cells; and

(ii) regenerating a plant from the transformed cells introduced with a vector in step (i) mentioned above.

As described above, the method of conferring clubroot resistance activity to plants or seeds thereof, which comprise the step of expressing a polynucleotide of the present invention in the cells of a plant, is also included in the present invention. For example, this method can be carried out by introducing a polynucleotide of the present invention or a vector of the present invention into plant cells. An example is a method comprising the following steps of:

(a) crossing a plant having a polynucleotide of the present invention with another plant; and

(b) selecting a plant having the aforementioned polynucleotide.

Plants or seeds thereof having clubroot resistance are produced by these methods. More specifically, by the methods of the present invention, for example, a plant sensitive to clubroot can be converted to a clubroot-resistant plant.

Plants that can be conferred resistance by methods of the present invention are not particularly limited, and resistance can be conferred to any plant. Examples include cruciferous plants. Specific examples include Chinese cabbage, turnip, Bok-choy, Nabana, Nozawa-na, Tsukena, cabbage, broccoli, cauliflower, rapeseed, daikon radish, and such, but are not limited thereto.

Furthermore, the present invention provides plants and seeds thereof produced by the above-mentioned methods of the present invention. For example, artificially produced plants or seeds thereof that carry polynucleotides of the present invention and have clubroot resistance activity are also included in the present invention.

Furthermore, the present invention provides oligonucleotides that have a chain length of at least 15 nucleotides and are complementary to the nucleotide sequence of SEQ ID NO: 1 or SEQ ID NO: 3 or their complementary sequences.

Herein, the term “complementary sequence” refers to the sequence of the other strand relative to the sequence of one strand in a double-stranded DNA consisting of A:T and G:C base pairs. Furthermore, the term “complementary” is not limited to the case of a completely complementary sequence in a region of at least 15 consecutive nucleotides, and may have at least 70%, preferably at least 80%, more preferably 90%, and even more preferably 95% or higher nucleotide sequence identity. Such DNAs are useful as probes for detecting the polynucleotides of the present invention or selecting plants (plant cells) having the polynucleotides of the present invention, or as primers for amplifying the polynucleotides of the present invention.

Specifically, the present invention provides primers and probes for detecting clubroot resistance activity in a test plant, which comprise an oligonucleotide having a chain length of at least 15 nucleotides and specifically hybridizes under stringent conditions with the nucleotide sequence of SEQ ID NO: 1.

Furthermore, the present invention provides reagents containing an oligonucleotide to be used in the methods for producing the plants of the present invention or seeds thereof. More specifically, reagents of the present invention contain the following oligonucleotides:

(a) oligonucleotide primers for amplifying the full-length sequences or partial sequences of the nucleotide sequences of SEQ ID NOs: 1 and 3; and

(b) oligonucleotide probes which have a chain length of at least 15 nucleotides and hybridize under stringent conditions with the nucleotide sequences of SEQ ID NOs: 1 and 3.

Primers or probes of the present invention can be synthesized by any method based on the nucleotide sequences constituting them. The length of the nucleotide sequence complementary to the genomic DNA of the primers or probes of the present invention is usually 15 to 100, generally 15 to 50, and preferably 15 to 30. Methods for synthesizing an oligonucleotide having the nucleotide sequence based on an obtained nucleotide sequence are well known. Furthermore, in oligonucleotide synthesis, an arbitrary modification can be introduced to an oligonucleotide using nucleotide derivatives modified with fluorescent dye, biotin and the like. Alternatively, methods for binding fluorescent dye or the like to synthetic oligonucleotides are also known.

Furthermore, the present invention provides a kit to be used in the various methods of the present invention. In preferred embodiments of the kits of the present invention, at least one type of oligonucleotide in (a) or (b) described above is included. Kits of the present invention may appropriately include in their package, positive or negative standard samples, instructions describing the method of use, and such.

Polynucleotides or vectors of the present invention can be used, for example, in the production of plants or seeds having clubroot fungus resistance activity. Plants or seeds thereof having clubroot fungus resistance activity can be produced by expressing in desired plants or seeds thereof polynucleotides or vectors of the present invention.

Therefore, the present invention relates to agents for conferring clubroot fungus resistance activity, which comprise a polynucleotide or vector of the present invention as the active ingredient. The term “agent for conferring clubroot fungus resistance activity” in the present invention refers to a pharmaceutical agent having the effect of conferring clubroot fungus resistance activity to all or a part of a plant or its seed, and refers to a substance or a composition (mixture) comprising a polynucleotide or a vector of the present invention as the active ingredient.

In the pharmaceutical agents of the present invention, in addition to polynucleotides or vectors which are the active ingredients, for example, sterilized water, physiological saline solution, plant oil, surfactants, lipids, solubilizing agents, buffers, preservatives, and such may be mixed in as necessary.

All prior art references cited in this specification are incorporated herein by reference.

### EXAMPLES

Hereinafter, the present invention is specifically described with reference to the Examples; however, the present invention should not be construed as being limited thereto. Reference examples have been disclosed (Suwabe, K., Tomita, N., Fukuoka, H., Suzuki, T., Mukai, Y, and Matsumoto, S., “Genetic refinement of the Chinese cabbage clubroot resistance gene locus Crr1 and synteny analysis with Arabidopsis thaliana using a physical map”, Breeding Research, supplement 1.2, (2005) 154).

### Reference Example 1

### Isolation of a BAC Library Carrying Crr1

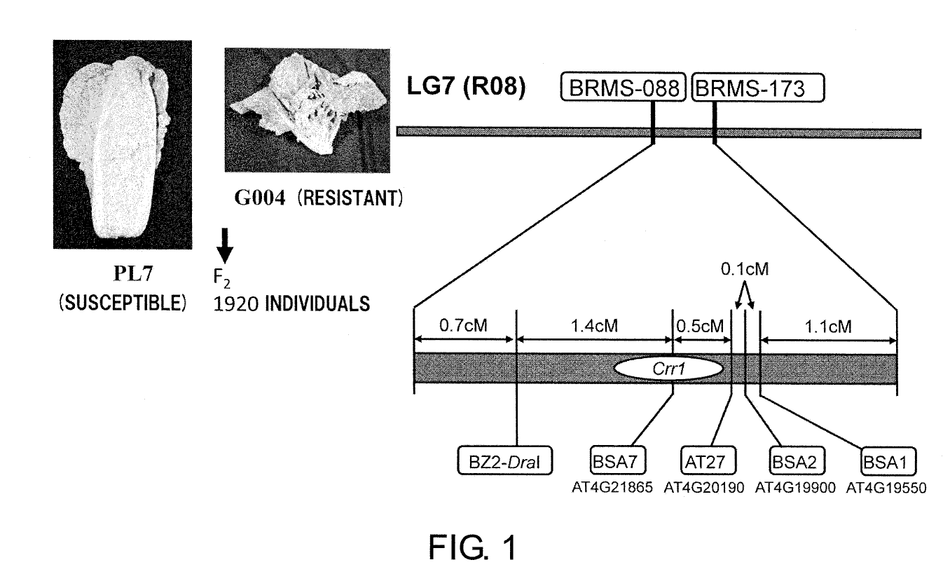

To isolate clubroot resistance genes by map-based cloning, a BAC library of resistant line G004 derived from the European fodder turnip “Siloga” was constructed. This library had an average insert length of 67.4 kb and a size of approximately 38,400 clones, and it was equivalent to 4.7-times the genome of B. rapa which is estimated to be 550 MB (Arumuganathan K, Earle E D., Plant Mol Biol Rep, (1991) 9 (3): 208-219). To isolate BAC clones carrying Crr1, chromosome walking was performed starting from BSA7, which is the marker most closely linked to Crr1 among the linked markers (FIG. 1, Suwabe, K. et al., Genetics, (2006) 173: 309-319).

After 96 arbitrary E. coli cells were selected and cultured overnight in a liquid medium, and replicas of E. coli were taken. These 96 E. coli were combined into one, and their plasmid DNA was extracted. 384 of such a plasmid DNA pool which combines 96 E. coli into one were produced. The first screening examined whether the BSA7 fragments contained in the 384 samples were amplified. For the pools in which amplification was confirmed by PCR, replicas of the initial 96 E. coli were cultured, and E. coli (clones) carrying the BSA7 sequence were identified by secondary screening. Plasmid of each clone was extracted, their terminal sequences were determined, and then PCR primers were designed using those terminal sequences as targets. Whether each terminal sequence was amplified was examined using the designed primers in all BAC clones possibly containing BSA7, and the manner in which each of the BAC clones overlapped was elucidated. From the BAC clones obtained this way, the BAC clone positioned at the very end was identified.

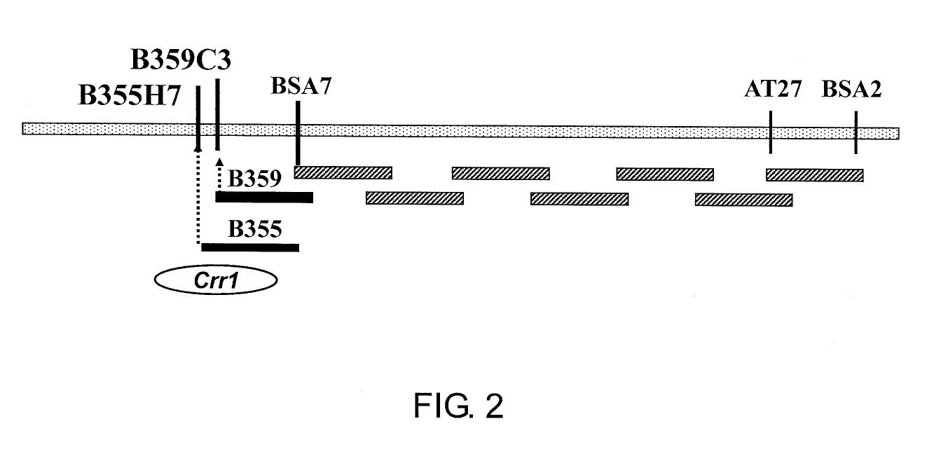

Primary screening was performed again using both terminal sequences of the identified BAC clone, and a number of BAC clones that overlapped starting from BSA7 were identified (FIG. 2). Furthermore, screening was repeated using the terminal sequences of the obtained clones, and ultimately BSA7 and BSA2 were covered. This region could be covered by a minimum of seven clones. Meanwhile, on the opposite side, B355H7 carrying the BSA7 marker sequence and a B359C3 clone that overlapped with it were obtained.

### Reference Example 2

### Narrowing Down the Location of Crr1 Using an Individual in which the Markers Near Crr1 are Recombined

Starting from BSA7, the region around Crr1 was covered with BAC clones, and by comparing the terminal sequences of these BACs between the susceptible cultivars PL7 and G004, multiple markers indicating polymorphism were obtained. From comparison of the marker genotype obtained from the terminal sequences of the BAC clones and the presence of the resistance gene, the markers produced from the terminal sequences of B355H7 and B359C3 were the markers closest to Crr1.

The marker genotypes of the G004 type and PL7 type were denoted by RR and rr, respectively, and the heterozygous type was denoted by Rr. To narrow down the Crr1 location, individuals with recombination between markers near Crr1 were screened from PL7, G004, and F₂ individuals. From approximately 5,700 F₂ individuals, those in which genomic recombination of the susceptible type and resistant type had taken place between BRMS-088 and BRMS-173 were selected. That is, three individuals (No. 1075, No. 764, and No. 572) in which recombination took place between markers of B355H7 and B359C3 were investigated in detail. The marker genotypes of each individual are as shown below.

| | B355H7 | B359C3 |
|---|---|---|
| F₂ No. 1075 | rr | Rr |
| F₂ No. 764 | Rr | rr |
| F₂ No. 572 | RR | Rr |

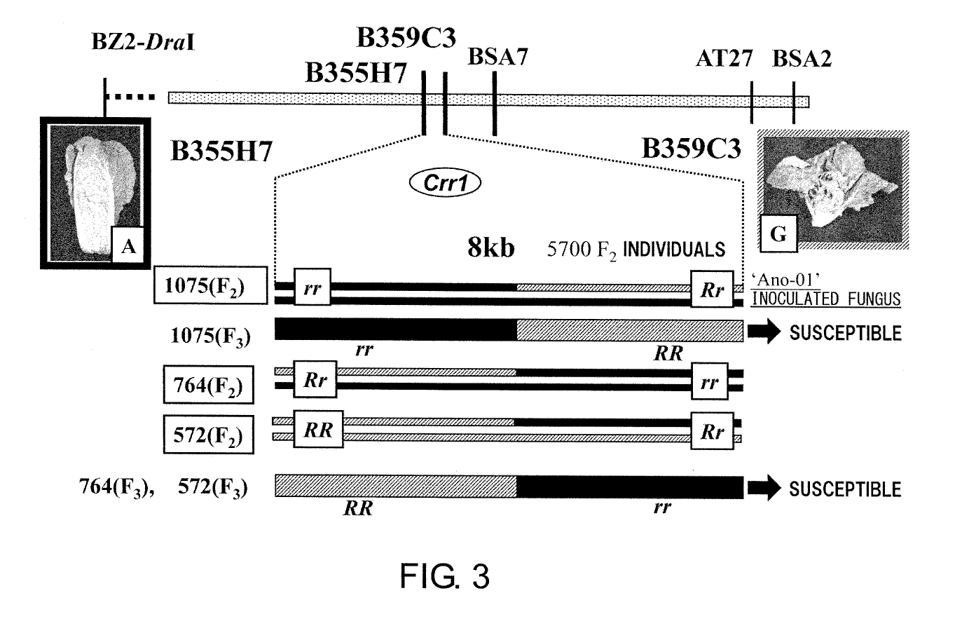

These three F₂ plants were self-reproduced to obtain F₃ seeds. The heterotype loci in the F₂ generation were separated into three types: RR, Rr, and rr in the F₃ generation. Clubroot resistance of the F₃ generation was assayed using the F₃ seeds and the clubroot fungus isolate “Ano-01”. Specifically, it was investigated whether plant individuals whose B355H7-B359C3 marker genotype is the rr-RR type in No. 1075, and the RR-rr-type in No. 764 and No. 572 are resistant (FIG. 3). Clubroot resistance assay was performed as described below according to the method of Yoshikawa et. al (Yoshikawa, H., Bull. Natl. Res. Inst. Veg., Ornam. Plants & Tea Japan, (1993) 7:1-465).

Horticultural culture soil was placed up to half the height from the bottom of a 9-cm-diameter jiffy pot, and a groove having a width of about 2 cm was made; and 10 g of infected soil, which was prepared so that 5×10⁶ resting spores of the clubroot fungus isolate “Ano-01” were present per 1 g of dry soil, was placed into the groove. Next, ten F₃ seeds were sown in each jiffy pot; and the plants were cultivated for six weeks in a phytotron with settings of approximately 20,000 lux illuminance, 16-hours day length, 23° C. temperature during the light period, and 18° C. temperature during the dark period. Then, the roots were washed. To evaluate the degree of damage of the roots, four levels of disease index were implemented in (0: knobs are not found at all; 1: tiny knobs are attached to the lateral roots; 2: serial knobs are attached to the lateral roots, or the symptoms are intermediate between damage levels 1 and 3; and 3: relatively large knobs are attached to the main root, or the main root is enlarged). Of the four stages, 0, 1, and 2 were judged to be “resistant” as there is certain resistance against clubroot; and 3 was judged to be “susceptible” with no resistance. Results of the resistance assay showed that none of the F₃ individuals of the three lineages had resistance, and their phenotype was determined to be the susceptible type.

Furthermore, DNA was extracted from each individual used in the resistance assay, the genotype of the nearby markers was determined, and the location of Crr1 was narrowed down based on the relationship between the marker genotypes and the presence of the resistance genes. In the F₃ individuals of No. 1075, individuals that have the RR-type B359C3 showed susceptibility. This strongly suggests that Crr1 does not exist towards the side of AT27 beyond B359C3, and the results with No. 764 and No. 572 strongly suggest that Crr1 does not exist towards the BZ2-DraI side beyond B355H7. Therefore, the resistance gene, Crr1, was estimated to be present within the approximately 8 kb region between B355H7 and B359C3 (FIG. 3).

### Example 1

### Estimation of the Crr1 Candidate Gene

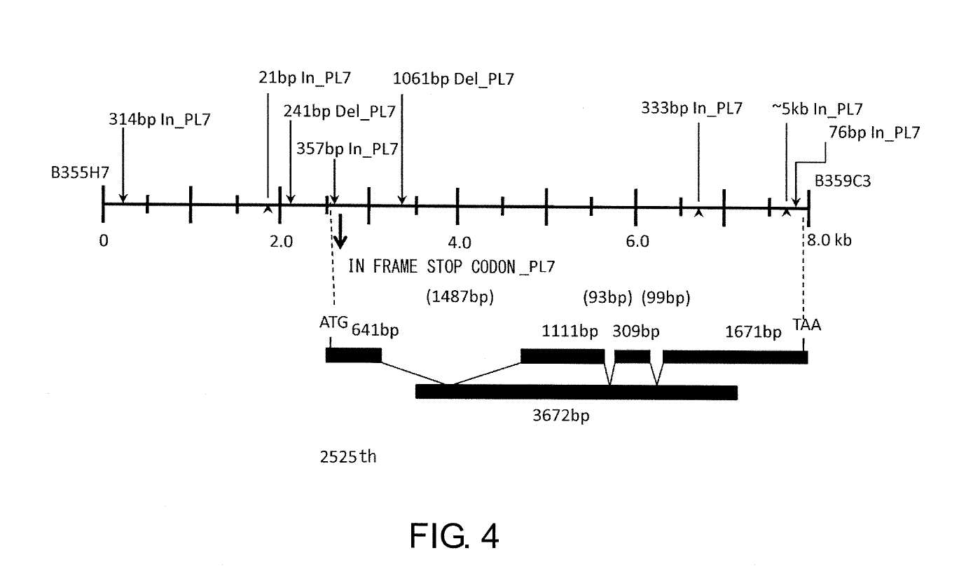

Shotgun clones of B355H7 were produced to determine the DNA sequence of the approximately 8-kb region between B355H7 and B359C3. The DNA sequences of the extracted plasmid fragments were determined according to a standard method using T7 and Reverse Primer, and a DNA Sequence Assembly Software, SEQUENCHER ver. 2 (Hitachi Software Engineering, Tokyo), was used to produce a single sequence. In this region, search of open reading frames (ORF) that encode proteins was done using a genetic information processing software, GENETYX (Genetyx, Tokyo). Based on the determined G004 sequence information, primers were designed at suitable positions, DNA fragments were amplified using the susceptible PL7 as a template, and the nucleotide sequence of this region in PL7 was determined. The DNA sequences were compared between PL7 and G004, and the inserted and deleted sequences as well as the presence of single nucleotide polymorphisms were investigated. As a result, the length of the DNA fragment between B355H7 and B359C3 in the resistant line G004 was 7,995 bp, and four ORFs were presumed to be present in this sequence (FIG. 4, SEQ ID NO: 3).

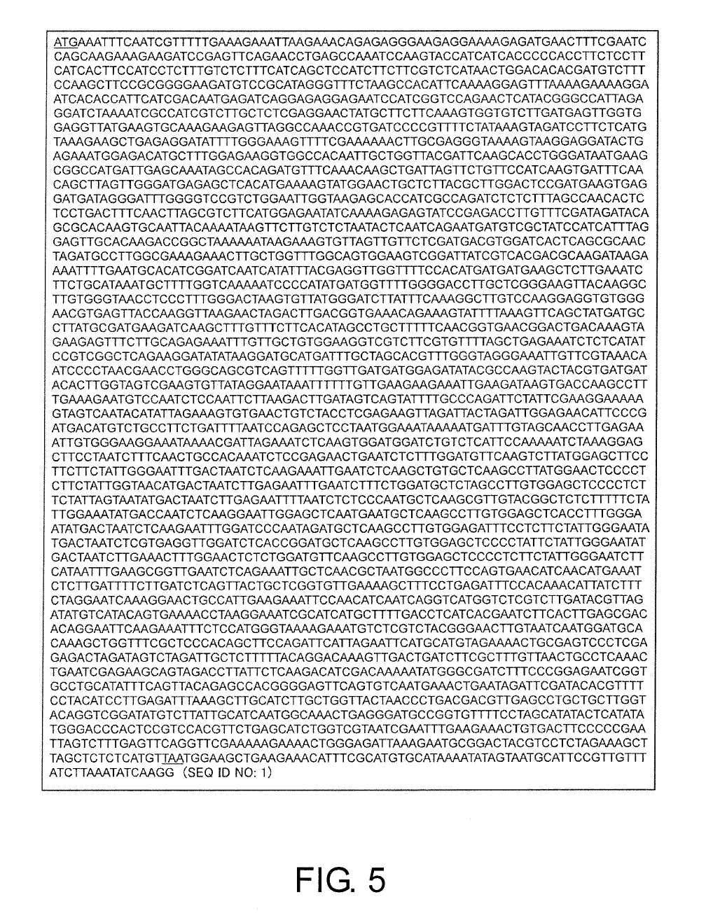

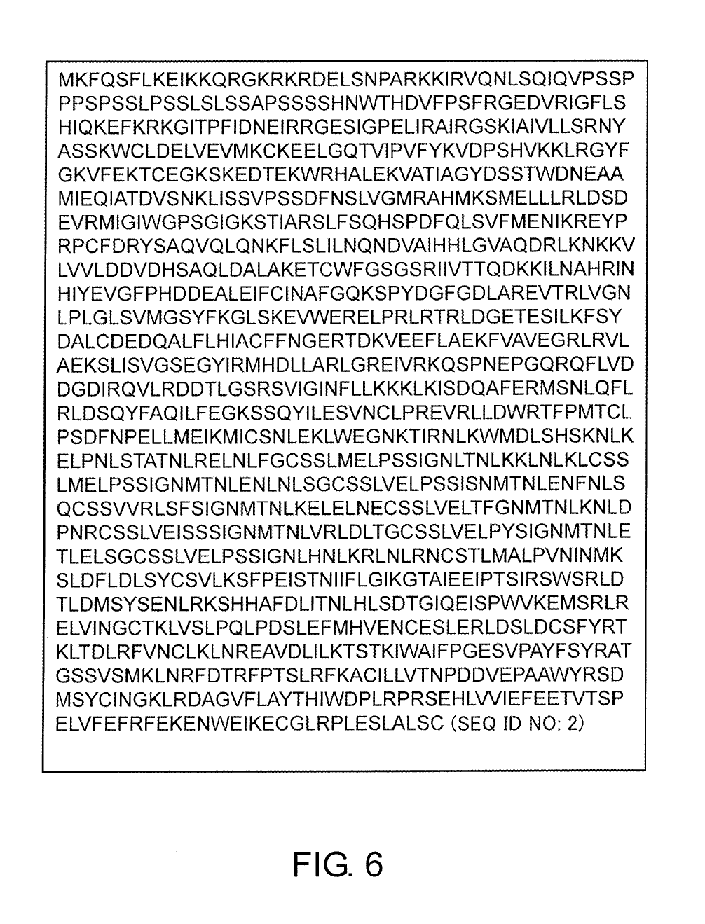

To investigate whether the estimated ORF regions are transcribed, a single-stranded cDNA was synthesized from poly(A)+ RNA extracted from the roots of resistant material An4-8-1 containing Crr1, then the primers, Crr1-Fsm (5′-TCC [CCCGGG] AAAATGAAATTTCAATCGTTTTTG-3′/SEQ ID NO:9; [ ] indicates the SmaI site) and Crr1-R (5′-CCTTGATATTTAAGATAAACAACGGAATG-3′/SEQ ID NO: 10) were used to amplify the cDNA region of the Crr1 gene. The cDNA nucleotide sequence was determined, and the amino acid translation region was estimated. As a result, a DNA fragment having nearly the same length as the fragment containing the four ORFs was found. The part starting from the ATG start codon first found in the nucleotide sequence of this cDNA to immediately before the TAA stop codon had 3672 bp, and was estimated to encode 1224 amino acids (FIG. 5, FIG. 6).

The genomic DNAs were compared to estimate the intron and exon portions. As a result, a structure in which four ORFs are linked into one by splicing was found. The splicing regions which separate the introns from the exons all followed the GT-AG rule.

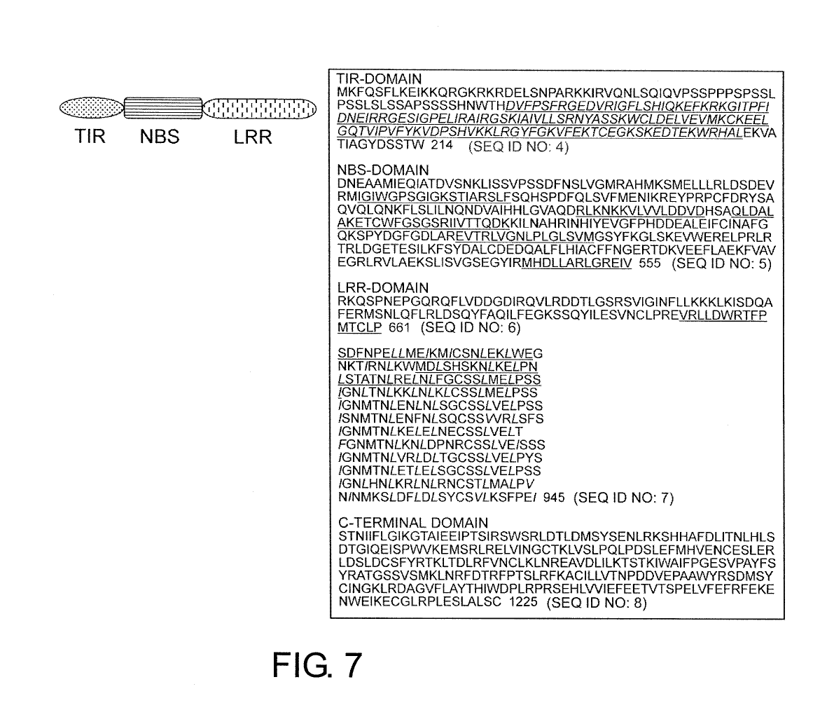

Furthermore, when the ends (the translation initiation and termination sites) of the transcription products were determined from the genomic sequences of resistant cultivars by performing 5′ race and 3′ race analyses, sequences encoding amino acids other than the four ORFs were not found, and this suggests that the four ORFs are transcribed as a single cDNA (SEQ ID NO: 3). The estimated amino acid sequence demonstrated the NIR-NBS-LRR structure which is a sequence commonly shared by disease resistant genes (FIG. 7).

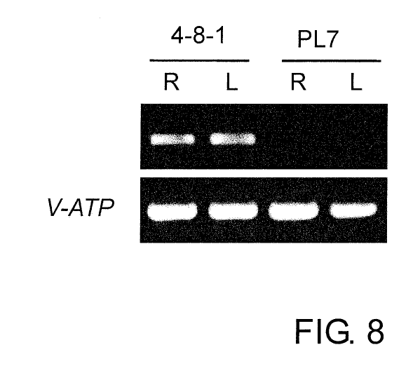

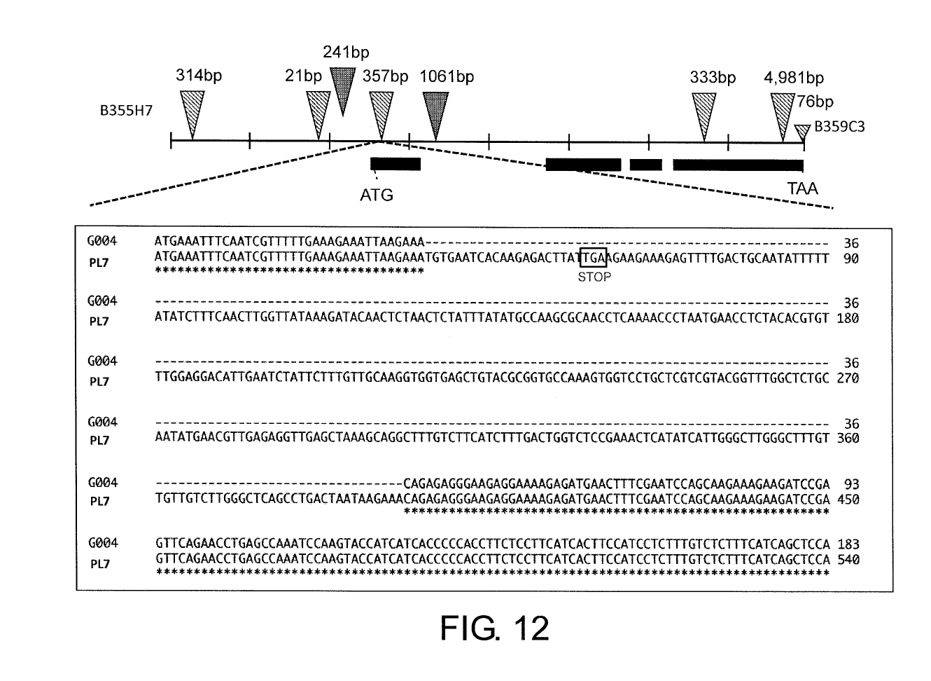

In comparison with this region in the susceptible cultivar PL7 (SEQ ID NO: 13), many insertion and deletion sequences and single nucleotide polymorphisms were found. Insertions and deletions of 100 bp or more were found in seven places. In particular, comparison of the 5′ side showed that insertion of 357 bp approximately 60 bp downstream of the start codon in PL7 placed an in-frame stop codon in exon 1 (FIG. 12), and on the 3′ side, a long insertion sequence of approximately 5 kb was present in PL7. Furthermore, the terminal sequence of B359C3 had a 78-bp insertion. Between them, 200 bp or longer exons were found in four places (SEQ ID NO: 3). The four estimated ORFs were defined with the region having a sequence of 641 bp as exon 1, and exons 1, 2, 3, and 4 in order. To investigate how the expression of the ORF regions differs between the resistant and susceptible cultivars, RT-PCR was performed by targeting the first exon regions in the resistant line An4-8-1 and the susceptible cultivar PL7. A primer set was designed using the sequences in exon 1. Using the cDNAs derived from the roots and leaves of the resistant line An4-8-1 and the susceptible PL7 as templates, amplification was carried out using the designed primers. V-ATP which is expressed constitutively in the cells was used as a positive control. As a result, amplification of DNAs at the intended chain length was confirmed in both of the roots and leaves in An4-8-1. However, in PL7, such amplification could not be confirmed in either the roots or the leaves. Therefore, the clubroot-resistance candidate gene was found to be expressed in the resistant line, but not expressed in the susceptible cultivar (FIG. 8).

### Example 2

### Proof of Crr1 by Complementarity Experiments

(1) Construction of Vectors for Introduction into Plant Cells

### Construction of a Vector Using the Lettuce Ubiquitin Gene as the Promoter

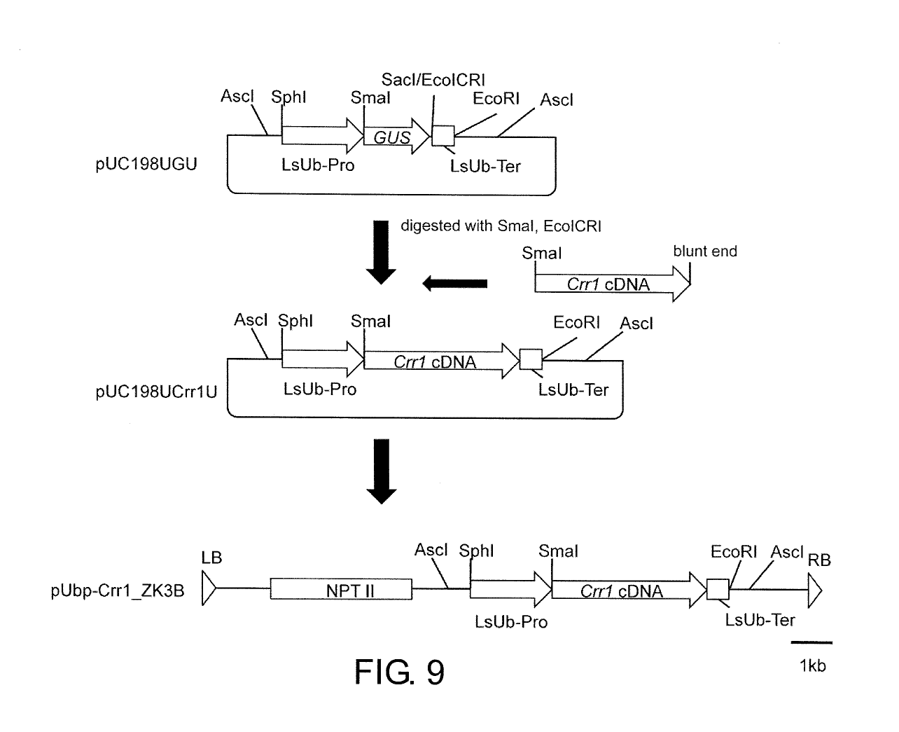

A plasmid pUC198UGU (received from Dr. H. Fukuoka) inserted with a GUS gene between the promoter and terminator of the ubiquitin gene cloned from lettuce was cleaved with SmaI and EcoICRI, and a Crr1 cDNA sequence treated in advance with SmaI was inserted to construct pUC198UCrr1U (FIG. 9). pUbp-Crr1_ZK3B was constructed by excising the lettuce ubiquitin promoter—Crr1 cDNA—lettuce ubiquitin terminator cassette using AscI, and inserting it into a binary vector pZK3B (received from Dr. M. Kuroda at the National Agriculture and Food Research Organization (NARO)/Agricultural Research Center (ARC)) which has been modified from pPZP202 (Hajdukiewicz P. et al., Plant Mol Biol, (1994) 25: 989-994) (FIG. 9).

The constructed binary vector was transformed into the agrobacterium GV3101 strain, and Arabidopsis thaliana Col-0 was transformed by the Flower-dip method. The seeds (T1) of Arabidopsis thaliana harvested from the agrobacterium-inoculated line (T0) carrying the constructed vector were seeded into a selection medium containing kanamycin, resistant individuals were selected, and a number of viable lines were obtained. Plants that can be cultivated on a selection medium were potted and self-propagated to obtain next-generation seeds (T2). These T2 seeds were tested under clubroot resistance assay.

(2) Clubroot Resistance Assay Using Transformed Arabidopsis thaliana

Clubroot assay of Arabidopsis thaliana was performed by modifying the method of Jubault M. et al., Theor. Appl. Genet., (2008) 117:191-202. Kanamycin-resistant plants were transferred to a horticultural culture soil (TM-1, TAKII) at nine individuals per pot, and three days later, 2 mL of a dormant spore solution (1.0×10⁶ spores/mL) was used to drench the base of the plant. The plants were cultivated under conditions of 22° C. and 14-hour day length; and three weeks after inoculation, the degree of symptoms at the root was investigated. The degree of disease development was evaluated at four levels: 0 (no symptoms); 1 (small knobs in the lateral roots); 2 (slightly large knobs in the lateral roots); and 3 (large knobs in the main root and enlargement of the hypocotyl).

As a result, when the untransformed columbia lines were subjected to clubroot fungus isolate Ano-01, out of the total 18 lines except for one, 17 lines showed marked root enlargement or thickening and were judged to be at level 3 in the disease index (Table 1: degrees of clubroot resistance in transformed Arabidopsis thaliana introduced with the Crr1 candidate gene).

TABLE 1

| | NUMBER OF EMERGED LINES | | | | | | NUMBER OF EMERGED LINES | | | | | |
|---|---|---|---|---|---|---|---|---|---|---|---|---|
| | ACCORDING TO | | | | RATIO | AVERAGE | ACCORDING TO | | | | RATIO | AVERAGE |
| NAME | THE DISEASE INDEX^{1),2)} | | | | OF DISEASED | DISEASE | THE DISEASE INDEX^{1),3)} | | | | OF DISEASED | DISEASE |
| OF LINE | 0 | 1 | 2 | 3 | LINES (%)⁴⁾ | INDEX⁵⁾ | 0 | 1 | 2 | 3 | LINES (%)⁴⁾ | INDEX⁵⁾ |
| Col-0 | 0 | 0 | 0 | 9 | 100.0 | 3.0 | 0 | 0 | 1 | 8 | 100.0 | 2.9 |
| (non- | | | | | | | | | | | | |
| transformant) | | | | | | | | | | | | |
| UpCrr1_02 | 9 | 0 | 0 | 0 | 0.0 | 0.0 | 9 | 0 | 0 | 0 | 0.0 | 0.0 |
| UpCrr1_04 | 9 | 0 | 0 | 0 | 0.0 | 0.0 | 8 | 1 | 0 | 0 | 11.1 | 0.1 |
| UpCrr1_09 | 2 | 4 | 1 | 0 | 71.4 | 0.9 | 5 | 2 | 0 | 2 | 44.4 | 0.9 |
| UpCrr1_11 | 4 | 5 | 0 | 0 | 55.6 | 0.6 | 4 | 1 | 1 | 3 | 55.6 | 1.3 |
| UpCrr1_15 | 6 | 3 | 0 | 0 | 33.3 | 0.3 | 7 | 0 | 0 | 2 | 22.2 | 0.7 |
| UpCrr1_17 | 9 | 0 | 0 | 0 | 0.0 | 0.0 | 9 | 0 | 0 | 0 | 0.0 | 0.0 |

¹⁾0 (no symptoms); 1 (small knobs in the lateral roots); 2 (slightly large knobs in the lateral roots); 3 (large knobs in the main root and enlargement of hypocotyl)
²⁾Date of inoculation: 2010.2.4; Date of examination: 2010.2.25-26
³⁾Date of inoculation: 2010.3.19; Date of examination: 2010.4.12
⁴⁾(total number of lines showing disease index 1, 2, or 3/total number of individuals) × 100
⁵⁾(number of lines with disease index 0 × 0 + number of lines with disease index 1 × 1 + number of lines with disease index 2 × 2 + number of lines with disease index 3 × 3)/total number of individuals

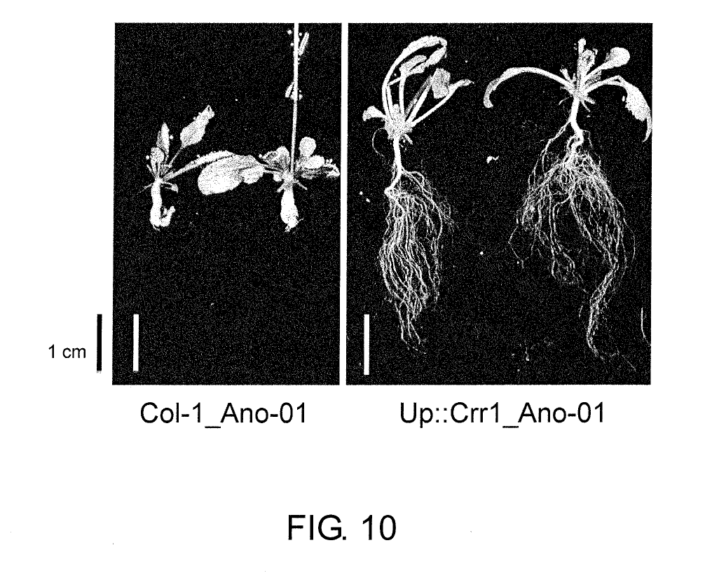

In these lines, a pigment presumed to be anthocyanin accumulated in the leaves, changing the color of the leaves to a pale red color, and the growth on the ground was markedly inhibited (FIG. 10). In two independent experiments, three lines of transformants, which are UpCrr1_{—}02, UpCrr1_{—}04, and UpCrr1_{—}17, produced large amounts of white healthy roots, the symptoms of clubroot were not observed at all, and the number of diseased lines as well as the disease index were both 0. Furthermore, the above-ground part also showed growth equivalent to the normally cultivated line, and it was evident that resistance against clubroot was clearly acquired. In the section that was inoculated on Mar. 19, 2010, although several lines judged to have disease index 3 were observed in some of UpCrr1_{—}09, UpCrr1_{—}11, and UpCrr1_{—}15, but there were more individuals with no disease development at all. When the candidate gene was used to transform the clubroot-susceptible Arabidopsis thaliana var. Columbia, clubroot-resistant Arabidopsis thaliana was obtained. From the above, the cloned gene was revealed to have a function of conferring clubroot resistance and was determined to be Crr1.

### Example 3

### Effect of Selection when Insertion/Deletion Sequences Found Between the Clubroot-Resistant and -Susceptible Lines were Used as Selection Markers

In marker selection using BRMS-173 and BRMS-088, recombination between the marker locus and the Crr1 locus took place in some individuals, albeit at a low probability. To avoid this, one can imagine using the resistance gene itself as marker. As shown in SEQ ID NO: 3, when the resistant and susceptible lines were compared for their Crr1 gene sequences, there were many insertion/deletion sequences. These can be used to examine their utility as markers for selecting resistant individuals.

Between the resistant line G004 and the susceptible cultivar PL7, an 86-bp insertion/deletion sequence is present near the C terminus of Crr1. This insertion/deletion sequence was used as a target, and the primer set B359C3 (forward primer: CTCTCTCATGTTAATGGAAGCTGA/SEQ ID NO: 11; and reverse primer: CACTCAACGAGTAGGAAACAAAGA/SEQ ID NO: 12) was constructed. The test material was an F₂ population derived by crossing the susceptible A line with “Hakusai Parental Line No. 9” (hereinafter, abbreviated as PL9) having two resistance genes, Crr1 and Crr2.

For the resistance assay, clubroot fungus isolates “Wakayama-01” and “No. 5” were inoculated. Since the “Wakayama-01” and “No. 5” isolates damage a considerably large number of Chinese cabbage cultivars, they are fungi with a wide host range. To confer resistance against “Wakayama-01” and “No. 5”, both clubroot resistance genes, Crr1 and Crr2, must be carried in homozygous forms (Suwabe, K. et al., Theor. Appl. Genet., (2003) 107: 997-1002).

Resistance assay was performed according to the method of Yoshikawa (1993) (Yoshikawa, H., Bull. Natl. Res. Inst. Veg., Ornam. Plants & Tea Japan, (1993) 7:1-465). The F₂ crossed seeds were seeded into diseased soils separately containing the “Wakayama-01” and “No. 5” fungi; and six weeks later, the disease index was determined based on the resistance indicator.

Furthermore, DNAs were extracted from all of the tested individuals, PCR was performed using the primers of B359C3 and BRMS-096, amplified fragments were fractionated by agarose gel electrophoresis, and three marker genotypes, i.e., resistant homozygous-type, heterozygous-type, and susceptible homozygous-type, were detected. Since Crr1 and Crr2 are located on separate linkage groups, the marker genotypes of B359C3 and BRMS-096 are independent. Therefore, these two marker genotypes were combined to produce six types of marker genotypes. The relationship between these six marker genotypes and their disease indices was carefully examined, and the utility of B359C3 as a marker was examined.

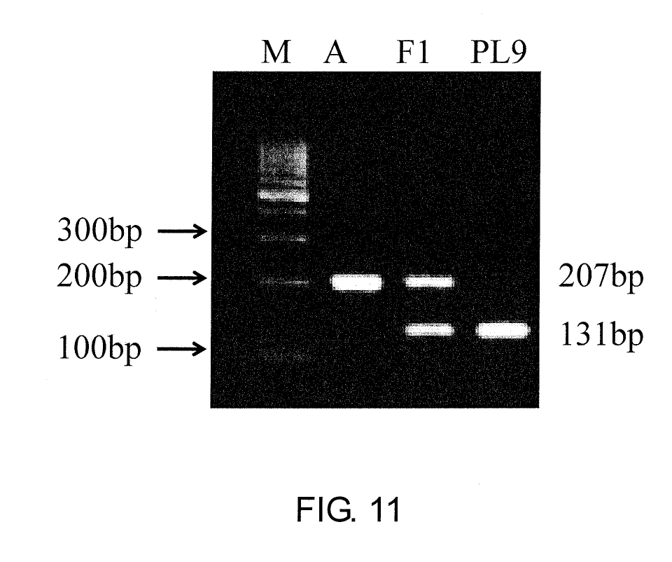

As a result, 131 bp were amplified in the resistant line PL9, whereas in the susceptible cultivar A, 207 bp containing a 68-bp insertion sequence was amplified. Furthermore, regarding BRMS-096 linked to Crr2, 220 bp were amplified in PL9 and 200 bp were amplified in the A line, and these differences were also clearly distinguishable by 2% agarose gel electrophoresis (FIG. 11). Data were obtained from 115 individuals inoculated with the Wakayama-01 isolate and 92 individuals inoculated with the No. 5 isolate. In the experiment using the Wakayama-01, among the 115 individuals, the number of individuals appearing to have resistance with a disease index of 0, 1, or 2 were 4, 7, and 6 individuals, respectively, which accounts for approximately ten percent of the whole. On the other hand, 98 individuals were susceptible to the disease, and this exceeded 90 percent. Apart from the disease index, when the number of emergences was examined by the marker genotype, of ten individuals that had the resistant homozygous type (RR,RR) as both of the two marker genotypes, 4, 3, and 3 had a disease index of 0, 1, or 2, respectively, and they were all resistant individuals. The average disease index was 0.9. The disease indices of the other five marker genotypes were mostly 3. Similar results were obtained in tests using the “No. 5” isolate.

Resistance against “Wakayama-01” and “No. 5” appeared only in individuals having both Crr1 and Crr2 in homozygous forms. B359C3 is a marker which has a 69-bp insertion/deletion sequence between the PL9 and A lines, and the PCR-amplified DNA fragments can be easily distinguished between the resistant line (PL9), the heterozygous individuals, and the susceptible line (PL7) by agarose gel electrophoresis. Furthermore, since BRMS-096 used to detect Crr2 mostly cosegregates with Crr2, individuals carrying the two markers in the resistant homozygous forms are predicted to show resistance. In fact, in experiments using “Wakayama-01” and “No. 5”, while more than 80% of the tested F₂ individuals were susceptible, individuals that have both of the two markers in the resistant homozygous type did not show susceptibility. Therefore, B359C3 was revealed to be a highly precise marker for selection of Crr1.

To date, there have been reports regarding detection of QTL related to clubroot resistance and markers linked to resistance genes, but when used as selection markers, there are two problems. The first one is that the distance between marker and the gene is close for use as selection marker. When the distance between the marker and the gene is 1 cM, breeders using the marker must be prepared to see recombination taking place in 1% or so in 100 individuals. BRMS-088 and BRMS-173, which are SSR markers linked to Crr1, were useful as selection markers, but since both of the markers are approximately 2 cM away from Crr1, even though the probability was low, emergence of recombinant individuals was a problem. Since Crr1 is located between BRMS-088 and BRMS-173, recombinant individuals could be excluded by using both markers. However, developing two markers that flank a gene of interest in this manner is not easy, except for crops whose entire nucleotide sequence has been disclosed such as rice. B359C3 allows amplification of the intron sequence of Crr1 bp PCR. Therefore, one hardly has to worry about the occurrence of recombinant individuals. The results are summarized in Table 2 (number of emerged individuals at each disease index in the inoculation test using the clubroot fungus isolate “Wakayama-01”) and Table 3 (number of emerged individuals at each disease index in the inoculation test using the clubroot fungus isolate “No. 5”), showing the relationship of the genotypes of the novel Crr1-linked marker B359C3 and the Crr2-linked marker BRMS-096 with the degree of resistance. Two experimental results support the findings.

TABLE 2

| | | | | | | | AVERAGE |
|---|---|---|---|---|---|---|---|
| | BRMS- | DISEASE INDEX*² | | | | | DISEASE |
| B359C3*¹ | 096*¹ | 0 | 1 | 2 | 3 | SUBTOTAL | INDEX*³ |
| rr | rr | 0 | 0 | 0 | 5 | 5 | 3.0 |
| rr | Rr | 0 | 0 | 0 | 9 | 9 | 3.0 |
| rr | RR | 0 | 0 | 1 | 3 | 4 | 2.8 |
| Rr | rr | 0 | 0 | 0 | 14 | 14 | 3.0 |
| Rr | Rr | 0 | 2 | 0 | 33 | 35 | 2.9 |
| Rr | RR | 0 | 2 | 0 | 11 | 13 | 2.7 |
| RR | rr | 0 | 0 | 0 | 12 | 12 | 3.0 |
| RR | Rr | 0 | 0 | 2 | 11 | 13 | 2.8 |
| RR | RR | 4 | 3 | 3 | 0 | 10 | 0.9 |

TABLE 3

| | | | | | | | AVERAGE |
|---|---|---|---|---|---|---|---|
| | BRMS- | DISEASE INDEX*² | | | | | DISEASE |
| B359C3*¹ | 096*¹ | 0 | 1 | 2 | 3 | SUBTOTAL | INDEX*³ |
| rr | rr | 0 | 0 | 0 | 5 | 5 | 3.0 |
| rr | Rr | 0 | 0 | 0 | 12 | 12 | 3.0 |
| rr | RR | 0 | 0 | 0 | 3 | 3 | 3.0 |
| Rr | rr | 0 | 0 | 0 | 12 | 12 | 3.0 |
| Rr | Rr | 0 | 1 | 1 | 21 | 23 | 2.9 |
| Rr | RR | 0 | 3 | 2 | 1 | 6 | 1.7 |
| RR | rr | 0 | 0 | 0 | 8 | 8 | 3.0 |
| RR | Rr | 1 | 0 | 2 | 12 | 15 | 2.7 |
| RR | RR | 3 | 4 | 1 | 0 | 8 | 0.8 |

The signs in Tables 2 and 3 above are shown below.
*¹Crr1 and Crr2 are located on separate chromosomes, and B359C3 and BRMS-096 are DNA markers that are linked to the Crr1 gene and the Crr2 gene, respectively. rr: susceptible parent, Rr: heterozygous type, RR: resistant parent type
*²disease indices; 0: no symptoms; 1: small knobs in the lateral roots; 2: serial knobs in the lateral roots; and 3: knobs in the main root
*³average disease index: Σ(disease index * number of individuals)/(total number of individuals)

Furthermore, it has been reported that there are genes around the gene of interest that are undesirable in terms of breeding, and their link could not be broken which results in linkage drag (Fukuoka S, et al., Science, (2009) 325(5943): 998-1001). When there is a linkage drag, the information of the region around the gene is necessary. At present, whether there is a gene that causes a linkage drag to Crr1 is unclear; however, if the sequence information of Crr1 is available, a linkage drag can be removed when present.

The second problem is that while the distance to the resistance gene is close, since the marker genotype of the individual carrying the gene of interest is the same as that of the individual to be introduced with the gene, it cannot be used as a marker. More specifically, depending on the susceptible cultivars and lineages, some may not carry Crr1 but may have marker genotypes completely identical to those of BRMS-088 and BRMS-173, and these cannot be used as selection markers. The gene region encoding Crr1 has been elucidated, and comparison with susceptible lines has become possible. Comparison of this region in resistant PL6 and susceptible PL7 showed many insertions/deletions and single nucleotide polymorphisms, and construction of markers has become efficient and easy. Even for cultivars and lines in which markers could not be used so far because they had the same marker genotype, it has become possible to construct markers easily by using the Crr1 region sequence as a reference. As described above, by using the Crr1 sequence, a marker producing polymorphism could be easily constructed regardless of the type of cultivar or lineage, and the obtained marker has a highly precise selection ability and practically does not produce any recombinant individuals.

### INDUSTRIAL APPLICABILITY

By the present invention, a clubroot resistance gene has been isolated, and it becomes possible to efficiently produce cruciferous plants with clubroot resistance by genetic recombination using the gene and marker selection. In particular, when performing marker selection, recombination between marker and gene does not occur when Crr1 or its surrounding sequences is used as marker; therefore, it has become possible to obtain a technique with very high selection effect. Furthermore, with common linkage markers, there are cases where marker selection may not be performed because the marker genotype coincides between the resistant and susceptible individuals. However, this time, since the entire nucleotide sequence of the resistance gene has been elucidated, differences in nucleotide sequence could be invariably found between the resistant and susceptible individuals, and development of markers became possible without difficulty. Accordingly, it is thought that there will be less need to perform cultivation that depends on chemosynthetic agrochemicals, and to rely on materials for promoting decrease of clubroot. Specifically, not only can the cost and labor to the farmers be decreased significantly, but continuous cropping will also become possible in the fields, and efficient farm management is expected to become possible. Furthermore, decrease in the use of chemosynthetic agrochemicals is desirable for the health of consumers as well.

1. A polynucleotide having clubroot fungus resistance, which is any one of (a) to (d) below:
(a) a polynucleotide encoding a protein comprising the amino acid sequence of SEQ ID NO: 2;
(b) a polynucleotide comprising the coding region of the nucleotide sequence of SEQ ID NO: 1;
(c) a polynucleotide encoding a protein comprising an amino acid sequence with one or more amino acid substitutions, deletions, additions, and/or insertions in the amino acid sequence of SEQ ID NO: 2; and
(d) a polynucleotide that hybridizes under stringent conditions with a complementary strand of the nucleotide sequence of SEQ ID NO: 1.

2. A vector in which the polynucleotide of claim 1 is operably linked downstream of a promoter region that enables expression in a plant cell.

3. A transformed plant cell into which the vector of claim 2 has been introduced.

4. A plant having clubroot fungus resistance activity, which is regenerated from the transformed cell of claim 3.

5. A plant having clubroot fungus resistance activity, which is a progeny or a clone of the plant of claim 4.

6. A propagation material of the plant having clubroot fungus resistance activity of claim 4.

7. A method for assessing clubroot fungus resistance of a test plant or a test propagation medium, which comprises the step of detecting a DNA region comprising the nucleotide sequence of SEQ ID NO: 1 or SEQ ID NO: 3, or a partial sequence or surrounding sequence of this nucleotide sequence.

8. The assessment method of claim 7, which comprises the following steps of:
(i) preparing a DNA sample from a test plant or a test propagation medium;
(ii) amplifying a DNA region comprising the nucleotide sequence of SEQ ID NO: 1 or SEQ ID NO: 3, or a partial sequence or surrounding sequence of this nucleotide sequence from the DNA sample; and
(iii) comparing the molecular weight or nucleotide sequence of a DNA fragment produced by amplifying the DNA region comprising the nucleotide sequence of SEQ ID NO: 1 or SEQ ID NO: 3, or a partial sequence or surrounding sequence of this nucleotide sequence, from the clubroot fungus-resistant plant or propagation medium with that of the DNA fragment amplified in step (ii).

9. A method of selecting a plant or seed thereof having a clubroot fungus resistance gene by the assessment method of claim 7.

10. A method for producing a plant or a seed thereof having clubroot fungus resistance activity, which comprises the following steps of:
(i) introducing into a plant cell the vector of claim 2; and
(ii) regenerating a plant from the transformed plant cell into which a vector has been introduced in step (i) mentioned above.

11. A method of conferring clubroot fungus resistance activity to a plant or seed thereof, which comprises the step of expressing the polynucleotide of claim 1 in a plant cell.

12. The method of claim 11, which comprises the step of introducing into a plant cell the polynucleotide or a vector in which the polynucleotide is operably linked downstream of a promoter region that enables expression in a plant cell.

13. The method of claim 11, which comprises the following steps of:
(a) crossing a plant comprising the polynucleotide with another plant; and
(b) selecting a plant comprising the polynucleotide.

14. The method of claim 7, wherein the plant is a cruciferous plant.

15. A plant or seed thereof which is obtained by the method of claim 7.

16. An artificially produced plant or seed thereof, which comprises the polynucleotide of claim 1 and has clubroot fungus resistance activity.

17. The plant or the seed thereof of claim 15, wherein the plant is a cruciferous plant.

18. A primer for detecting clubroot fungus resistance activity of a test plant, which comprises an oligonucleotide having a chain length of at least 15 nucleotides, and specifically hybridizes under stringent conditions with the nucleotide sequence of SEQ ID NO: 1 or the nucleotide sequence of SEQ ID NO: 3.

19. A probe for detecting clubroot fungus resistance activity of a test plant, which comprises an oligonucleotide having a chain length of at least 15 nucleotides, and specifically hybridizes under stringent conditions with the nucleotide sequence of SEQ ID NO: 1 or the nucleotide sequence of SEQ ID NO: 3.

20. A propagation material of the plant having clubroot fungus resistance activity of claim 5.

21. The method of claim 9, wherein the plant is a cruciferous plant.

22. The method of claim 10, wherein the plant is a cruciferous plant.

23. The method of claim 11, wherein the plant is a cruciferous plant.

24. A plant or seed thereof which is obtained by the method of claim 9.

25. A plant or seed thereof which is obtained by the method of claim 10.

26. A plant or seed thereof which is obtained by the method of claim 11.

27. The plant or the seed thereof of claim 16, wherein the plant is a cruciferous plant.

28. The plant or the seed thereof of claim 24, wherein the plant is a cruciferous plant.

29. The plant or the seed thereof of claim 25, wherein the plant is a cruciferous plant.

30. The plant or the seed thereof of claim 26, wherein the plant is a cruciferous plant.
